SEQUENCE LISTING (304 sequences)

SEQ ID NO: 1 - DNA, 426 bp, Homo sapiens
aacatccagaatccggaccccgccgtgtaccagctgagagacagcaagagcagcgacaacactgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaatttccagaacctgagcgtgatcggcttcagaatcctgctgctgaaggtggccggcttcaacctgctgatgaccctgcggctgtggtccagctga

SEQ ID NO: 2 - DNA, 426 bp, Homo sapiens
aacatccagaatccggaccccgccgtgtaccagctgagagacagcaagagcagcgacaagagtgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaatttccagaacctgagcgtgatcggcttcagaatcctgctgctgaaggtggccggcttcaacctgctgatgaccctgcggctgtggtccagctga

SEQ ID NO: 3 - DNA, 540 bp, Homo sapiens
gaggatctgaagaatgttttccccccggaagtgaccgtgttcgaaccaagcgaagccgagatatcccatacacagaaggctacattggtgtgcctggcgactggattttacccagaccacgtagaactgagctggtgggtgaacgggaaggaagtacattctggagtgtgcacagatccgcagcctctcaaagagcagccagcactgaatgactcccgatactgcctttcctcccgcctccgcgtgtcagctacattctggcagaatccaagaaatcattttagatgtcaggtgcagttctacggtcttagtgaaaacgacgagtggacccaggacagggcaaagccagtcacgcagattgtgtccgctgaagcctggggcagggccgactgcggcttcacaagcgagtcataccagcagggggtcctcagcgccacaattttgtacgagatactgctgggaaaggcaaccctctacgcggtgctggtttcagcccttgtcctgatggccatggtcaagcgaaaagactctcgagggtaa

SEQ ID NO: 4 - DNA, 540 bp, Homo sapiens
gaagatctgaagaacgtgttccccccagaggtgaccgtgttcgagcctagcgaggccgagatcagccacacccagaaagccaccctcgtgtgcctggccaccggcttctatcccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccggggctga

SEQ ID NO: 5 - DNA, 540 bp, Homo sapiens
gaagatctgaagaacgtgttccccccagaggtggccgtgttcgagcctagcgaggccgagatcagccacacccagaaagccaccctcgtgtgcctggccaccggcttctatcccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccggggctga

SEQ ID NO: 6 - DNA, 414 bp, Mus musculus
gacatccagaaccccgagcccgccgtgtaccagctgaaggaccccagaagccaggacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctga

SEQ ID NO: 7 - DNA, 522 bp, Mus musculus
gaagatctgaggaacgtgaccccccccaaggtgaccctgttcgagcccagcaaggccgagatcgccaacaagcagaaagccaccctggtctgcctggccaggggcttcttccccgaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagctga

SEQ ID NO: 8 - DNA, 315 bp, Homo sapiens
atgtggggagcctttctgctgtacgtgtccatgaagatgggcggcacagccggccagagcctggaacagcctagcgaagtgaccgccgtggaaggcgccatcgtgcagatcaactgcacctaccagaccagcggcttctacggcctgagctggtatcagcagcacgacggcggagcccccaccttcctgagctacaacgccctggacggcctggaagagacaggccggttcagcagcttcctgagcagaagcgacagctacggctacctgctgctgcaggaactgcagatgaaggacagcgccagctacttctgc

SEQ ID NO: 9 - DNA, 315 bp, Homo sapiens
atgtggggagtgtttctgctgtacgtgtccatgaagatgggcggcaccaccggccagaacatcgaccagcccaccgagatgaccgccaccgagggcgccatcgtgcagatcaactgcacctaccagaccagcggcttcaacggcctgttctggtatcagcagcacgccggcgaggcccccaccttcctgagctacaacgtgctggacggcctggaagagaagggccggttcagcagcttcctgagcagaagcaagggctacagctacctgctgctgaaagaactgcagatgaaggacagcgccagctacctgtgc

SEQ ID NO: 10 - DNA, 327 bp, Homo sapiens
atggccctgcagagcaccctgggcgccgtgtggctgggcctgctgctgaacagcctgtggaaggtggccgagagcaaggaccaggtgttccagcccagcaccgtggccagcagcgagggcgccgtggtggagatcttctgcaaccacagcgttagcaacgcctacaacttcttctggtacctgcacttccccggctgcgccccccgcctgctggtgaagggcagcaagcccagccagcagggccgctacaacatgacctacgagcgcttcagcagcagcctgctgatcctgcaggtgcgcgaggccgacgccgccgtgtactactgc

SEQ ID NO: 11 - DNA, 330 bp, Homo sapiens
atggcctctgcccctatcagcatgctggccatgctgttcaccctgagcggcctgagagcccagagcgtggcccagcccgaggatcaagtcaacgtggccgagggcaaccccctgaccgtgaagtgcacctacagcgtgtccggcaacccctacctgttttggtacgtgcagtaccccaaccggggcctgcagttcctgctgaagtacatcaccggcgacaacctggtgaaaggcagctacggcttcgaggccgagttcaacaagagccagaccagcttccacctgaagaaacccagcgccctggtgtccgacagcgccctgtacttctgc

SEQ ID NO: 12 - DNA, 315 bp, Homo sapiens
atgcgccaggtggcccgcgtgatcgtgttcctgaccctgagcaccctgagcctggccaagaccacccagcccatcagcatggacagctacgagggccaggaggtgaacatcacctgcagccacaacaacatcgccaccaacgactacatcacctggtaccagcagttccccagccagggcccccgcttcatcatccagggctacaagaccaaggtgaccaacgaggtggccagcctgttcatccccgccgaccgcaagagcagcaccctgagcctgccccgcgtgagcctgagcgacaccgccgtgtactactgc

SEQ ID NO: 13 - DNA, 330 bp, Homo sapiens
atgaaaaccttcgccggcttcagcttcctgttcctgtggctgcagctggactgcatgagcagaggcgaggacgtggaacagagcctgttcctgagcgtgcgcgagggcgacagcagcgtgatcaactgcacctacaccgacagcagcagcacctacctgtactggtacaagcaggaacccggcgctggcctgcagctgctgacctacatcttcagcaacatggacatgaagcaggaccagcggctgaccgtgctgctgaacaagaaggacaagcacctgagcctgcggatcgccgacacccagaccggcgacagcgccatctacttttgc

SEQ ID NO: 14 - DNA, 330 bp, Homo sapiens
atggaatcttttctgggcggcgtgctgctgatcctgtggctgcaggtcgactgggtcaagagccagaagatcgagcagaacagcgaggccctgaacatccaggaaggcaagaccgccaccctgacctgcaactacaccaactacagccccgcctacctgcagtggtacagacaggaccccggcagaggccccgtgttcctgctgctgattcgcgagaacgagaaagagaagcgcaaagagcggctgaaagtcaccttcgacaccaccctgaagcagagcctgttccacatcaccgccagccagcccgccgacagcgccacatatctgtgc

SEQ ID NO: 15 - DNA, 327 bp, Homo sapiens
atggaaaagatgcggaggcccgtgctgatcatcttctgcctgtgcctgggctgggccaacggcgagaaccaggtggaacacagcccccactttctgggcccccagcagggggatgtggccagcatgagctgcacctacagcgtgtcccggttcaacaacctgcagtggtacagacagaacaccggcatgggccccaaacatctgctgagcatgtacagcgccggctacgagaagcagaagggccggctgaacgccaccctgctgaagaacggcagcagcctgtacatcaccgccgtgcagcccgaggacagcgccacctacttttgc

SEQ ID NO: 16 - DNA, 330 bp, Homo sapiens
atgctgctgctgctcatccccgtgctgggcatgatcttcgccctgcgggatgccagagcccagagcgtgtcccagcacaaccaccacgtcatcctgagcgaggccgccagcctggaactgggctgcaactacagctacggcggcaccgtgaacctgttttggtacgtgcagtaccccggccagcatctgcagctgctgctgaagtacttctccggcgaccccctggtcaagggcatcaagggcttcgaggccgagttcatcaagagcaagttcagcttcaacctgcggaagcccagcgtgcagtggtccgataccgccgagtacttctgc

SEQ ID NO: 17 - DNA, 330 bp, Homo sapiens
atgctgctgctgctcgtgcccgtgctggaagtgatcttcaccctgggcggcaccagagcccagagcgtgacccagctggacagccatgtgtccgtgtccgagggcacccccgtgctgctgcggtgcaactacagcagcagctacagccccagcctgttttggtacgtgcagcaccccaacaagggcctgcagctgctgctgaagtacaccagcgccgccaccctggtcaagggcatcaacggcttcgaggccgagttcaagaagtccgagacaagcttccacctgaccaagcccagcgcccacatgagcgacgccgccgagtacttctgc

SEQ ID NO: 18 - DNA, 330 bp, Homo sapiens
atgctgctggaactgatccccctgctgggcatccatttcgtgctgcggaccgccagagcccagagcgtgacccagcccgacatccacatcaccgtgtccgagggcgccagcctggaactgcggtgcaactacagctacggcgccaccccctacctgttttggtacgtgcagagcccaggccagggcctgcagctgctgctgaagtacttctccggcgacaccctggtgcagggcatcaagggcttcgaggccgagttcaagcggagccagagcagcttcaacctgcggaagccctccgtgcattggagcgacgccgccgagtacttctgc

SEQ ID NO: 19 - DNA, 330 bp, Homo sapiens
atgctgctgctgctggtgcccgtgctggaggtgatcttcaccctgggcggcacccgcgcccagagcgtgacccagctgggcagccacgtgagcgtgagcgagggcgccctggtgctgctgcgctgcaactacagcagcagcgtgcccccctacctgttctggtacgtgcagtaccccaaccagggcctgcagctgctgctgaagtacaccagcgccgccaccctggtgaagggcatcaacggcttcgaggccgagttcaagaagagcgagaccagcttccacctgaccaagcccagcgcccacatgagcgacgccgccgagtacttctgc

SEQ ID NO: 20 - DNA, 330 bp, Homo sapiens
atgctgctgctgctcgtgcctgccttccaggtcatcttcaccctgggcggcaccagagcccagagcgtgacccagctggatagccaggtgcccgtgttcgaggaagcccccgtcgagctgcggtgcaactacagcagcagcgtgtccgtgtacctgttttggtacgtgcagtaccccaaccagggcctgcagctgctgctgaagtacctgagcggcagcaccctggtggaatccatcaacggcttcgaggccgagttcaacaagagccagaccagcttccacctgagaaagcccagcgtgcacatcagcgataccgccgagtacttctgc

SEQ ID NO: 21 - DNA, 327 bp, Homo sapiens
atgaattctagccctggccccgctatcgccctgttcctgatgttcggcggcatcaacggcgacagcgtggtgcagacagagggccaggtgctgcccagcgagggcgacagcctgatcgtgaactgcagctacgagacaacccagtaccccagcctgttttggtacgtgcagtaccccggcgagggcccccagctgcacctgaaagccatgaaggccaacgacaagggccggaacaagggcttcgaggccatgtaccggaaagagacaaccagcttccacctggaaaaggacagcgtgcaggaaagcgacagcgccgtctacttctgc

SEQ ID NO: 22 - DNA, 327 bp, Homo sapiens
atgaactacagccccggcctggtgagcctgatcctgctgctgctgggccgcacccgcggcaacagcgtgacccagatggagggccccgtgaccctgagcgaggaggccttcctgaccatcaactgcacctacaccgccaccggctaccccagcctgttctggtacgtgcagtaccccggcgagggcctgcagctgctgctgaaggccaccaaggccgacgacaagggcagcaacaagggcttcgaggccacctaccgcaaggagaccaccagcttccacctggagaagggcagcgtgcaggtgagcgacagcgccgtgtacttctgc

SEQ ID NO: 23 - DNA, 333 bp, Homo sapiens
atgaagaagcacctgaccacctttctggtcatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccccgtgtccctgaccatcatgaccttcagcgagaacaccaagagcaacggccggtacaccgccaccctggacgccgacacaaagcagagcagcctgcacatcaccgccagccagctgagcgacagcgccagctacatctgc

SEQ ID NO: 24 - DNA, 327 bp, Homo sapiens
atgatcagcctgagagtgctgctggtcatcctgtggctgcagctgagctgggtctggtcccagcggaaagaggtggaacaggaccctggccccttcaacgtgccagagggcgccaccgtggccttcaactgcacctacagcaacagcgccagccagagcttcttctggtacagacaggactgccggaaagaacccaagctgctgatgagcgtgtacagcagcggcaacgaggacggccggttcaccgcccagctgaacagagcctcccagtacatcagcctgctgatccgggacagcaagctgagcgacagcgccacctacctgtgc

SEQ ID NO: 25 - DNA, 330 bp, Homo sapiens
atgaagagcctgcgcgtgctgctggtgatcctgtggctgcagctgagctgggtgtggagccagcagaaggaggtggagcagaacagcggccccctgagcgtgcccgagggcgccatcgccagcctgaactgcacctacagcgaccgcggcagccagagcttcttctggtaccgccagtacagcggcaagagccccgagctgatcatgttcatctacagcaacggcgacaaggaggacggccgcttcaccgcccagctgaacaaggccagccagtacgtgagcctgctgatccgcgacagccagcccagcgacagcgccacctacctgtgc

SEQ ID NO: 26 - DNA, 333 bp, Homo sapiens
atgatgaagtccctgcgggtgctgctggtcatcctgtggctgcagctgagctgggtctggtcccagcagaaagaggtggaacaggaccctggccccctgagcgtgccagagggcgccatcgtgtccctgaactgcacctacagcaacagcgccttccagtacttcatgtggtacagacagtacagccggaagggccccgagctgctgatgtacacctacagctccggcaacaaagaggacggccggttcaccgcccaggtggacaagagcagcaagtacatcagcctgttcatccgggacagccagcccagcgacagcgccacctatctgtgc

SEQ ID NO: 27 - DNA, 327 bp, Homo sapiens
atgacctctatccgggccgtgttcatcttcctgtggctgcagctggacctggtcaacggcgagaacgtggaacagcaccccagcaccctgagcgtgcaggaaggcgacagcgccgtcatcaagtgcacctacagcgactccgccagcaactacttcccctggtacaagcaggaactgggcaagggcccccagctgatcatcgacatccggtccaacgtgggcgagaagaaggaccagcggatcgccgtgaccctgaacaagaccgccaagcacttcagcctgcacatcaccgagacacagcccgaggactccgccgtgtacttctgc

SEQ ID NO: 28 - DNA, 330 bp, Homo sapiens
atggctggaatccgggccctgttcatgtacctgtggctgcagctggactgggtgtccagaggcgagagcgtgggcctgcatctgcccaccctgagcgtgcaggaaggcgacaacagcatcatcaactgcgcctacagcaacagcgccagcgactacttcatctggtacaagcaggaaagcggcaagggcccccagttcatcatcgacatccggtccaacatggacaagcggcagggccagcgcgtgaccgtgctgctgaacaagaccgtgaagcacctgagcctgcagatcgccgccacccagcctggcgatagcgccgtgtacttctgc

SEQ ID NO: 29 - DNA, 336 bp, Homo sapiens
atgtctctgagcagcctgctgaaggtcgtgaccgccagcctgtggctgggccctggaatcgcccagaagatcacccagacccagcccggcatgttcgtgcaggaaaaagaggccgtcaccctggactgcacctacgacaccagcgaccccagctacggcctgttctggtacaagcagcccagcagcggcgagatgatcttcctgatctaccagggcagctacgaccagcagaacgccaccgagggccggtacagcctgaacttccagaaggcccggaagtccgccaacctggtcatcagcgccagccagctgggcgacagcgccatgtacttttgc

SEQ ID NO: 30 - DNA, 318 bp, Homo sapiens
atgaagcctaccctgatcagcgtgctggtcatcatcttcatcctgcggggcaccagagcccagagagtgacccagcccgagaagctgctgagcgtgttcaagggcgctcccgtggaactgaagtgcaactacagctacagcggcagccccgagctgttttggtacgtgcagtacagccggcagcggctgcagctgctcctgcggcacatcagcagagagagcatcaagggcttcaccgccgacctgaacaagggcgagacaagcttccacctgaagaagcccttcgcccaggaagaggacagcgccatgtactactgc

SEQ ID NO: 31 - DNA, 327 bp, Homo sapiens
atggagaccctgctgggcgtgagcctggtgatcctgtggctgcagctggcccgcgtgaacagccagcagggcgaggaggacccccaggccctgagcatccaggagggcgagaacgccaccatgaactgcagctacaagaccagcatcaacaacctgcagtggtaccgccagaacagcggcaggggcctggtgcacctgatcctgatccgcagcaacgagcgcgagaagcacagcggcaggctgcgcgtgaccctggacaccagcaagaagagcagcagcctgctgatcaccgccagccgcgccgccgacaccgccagctacttctgc

SEQ ID NO: 32 - DNA, 324 bp, Homo sapiens
atgctgagcgcctcttgtagcggcctggtcatcctgctgatcttccggcggaccagcggcgacagcgtgacccagactgagggccctgtgaccctgcctgagagagccgccctgaccctgaactgcacctaccagagcagctacagcaccttcctgttttggtacgtgcagtacctgaacaaagagcccgagctgctgctgaagtccagcgagaaccaggaaaccgacagccggggcttccaggccagccccatcaagagcgacagcagcttccacctggaaaagcccagcgtgcagctgagcgacagcgccgtgtactactgc

SEQ ID NO: 33 - DNA, 336 bp, Homo sapiens
atgctgaccgccagcctgctgcgcgccgtgatcgccagcatctgcgtggtgagcagcatggcccagaaggtgacccaggcccagaccgagatcagcgtggtggagaaggaggacgtgaccctggactgcgtgtacgagacccgcgacaccacctactacctgttctggtacaagcagccccccagcggcgagctggtgttcctgatccgccgcaacagcttcgacgagcagaacgagatcagcggccgttacagctggaacttccagaagagcaccagcagcttcaacttcaccatcaccgccagccaggtggtggacagcgccgtgtacttctgc

SEQ ID NO: 34 - DNA, 327 bp, Homo sapiens
atggaaaagatgctggaatgcgccttcatcgtgctgtggctgcagctgggctggctgagcggcgaggaccaggtcacacagagccccgaggccctgagactgcaggaaggcgagagcagcagcctgaactgcagctacaccgtgtccggcctgcggggcctgttctggtacagacaggaccccggcaagggccccgagttcctgttcaccctgtactctgccggcgaggaaaaagagaaagagcggctgaaggccaccctgaccaagaaagagagcttcctgcacatcaccgcccccaagcccgaggacagcgccacctatctgtgc

SEQ ID NO: 35 - DNA, 327 bp, Homo sapiens
atggagaccctgctgggcctgctgatcctgtggctgcagctgcagtgggtgagcagcaagcaggaggtgacccagatccccgccgccctgagcgtgcccgagggcgagaacctggtgctgaactgcagcttcaccgacagcgccatctacaacctgcagtggttccgccaggaccccggcaagggcctgaccagcctgctgctgatccagagcagccagcgcgagcagaccagcggccgcctgaacgccagcctggacaagagcagcggccgcagcaccctgtacatcgccgccagccagcccggcgacagcgccacctacctgtgc

SEQ ID NO: 36 - DNA, 321 bp, Homo sapiens
atgaagagaatcctgggcgccctgctgggcctgctgtctgcccaggtctgctgtgtgcggggcatccaggtggaacagagcccccctgacctgatcctgcaggaaggcgccaacagcaccctgcggtgcaacttcagcgacagcgtgaacaacctgcagtggttccaccagaacccctggggccagctgatcaacctgttctacatccccagcggcaccaagcagaacggccggctgagcgccaccaccgtggccaccgagagatacagcctgctgtacatcagcagcagccagaccaccgacagcggcgtgtacttctgc

SEQ ID NO: 37 - DNA, 354 bp, Homo sapiens
atggataagatcctgggcgccagcttcctggtgctgtggctgcagctgtgctgggtgtccggccagcagaaagagaagtccgaccagcagcaggtcaaacagagcccccagagcctgatcgtgcagaagggcggcatcagcatcatcaactgcgcctacgagaataccgccttcgactacttcccctggtatcagcagttccccggcaagggccctgccctgctgatcgccatcagacccgacgtgtccgagaagaaagagggccggttcaccatcagcttcaacaagagcgccaagcagttcagcctgcacatcatggacagccagcccggcgacagcgccacctacttttgc

SEQ ID NO: 38 - DNA, 336 bp, Homo sapiens
atggagaagaaccccctggccgcccccctgctgatcctgtggttccacctggactgcgtgagcagcatcctgaacgtggagcagagcccccagagcctgcacgtgcaggagggcgacagcaccaacttcacctgcagcttccccagcagcaacttctacgccctgcactggtaccgctgggagaccgccaagagccccgaggccctgttcgtgatgaccctgaacggcgacgagaagaagaagggccgcatcagcgccaccctgaacaccaaggagggctacagctacctgtacatcaagggcagccagcccgaggacagcgccacctacctgtgc

SEQ ID NO: 39 - DNA, 321 bp, Homo sapiens
atgctgctgatcacctccatgctggtgctgtggatgcagctgagccaggtcaacggccagcaggtcatgcagatcccccagtaccagcatgtgcaggaaggcgaggacttcaccacctactgcaacagcagcaccaccctgagcaacatccagtggtacaagcagcggcctggcggccatcccgtgtttctgatccagctggtcaagtccggcgaagtgaagaagcagaagcggctgaccttccagttcggcgaggccaagaagaacagcagcctgcacatcaccgccacccagaccaccgacgtgggcacctacttttgc

SEQ ID NO: 40 - DNA, 315 bp, Homo sapiens
atgagactggtggccagagtgaccgtgttcctgaccttcggcaccatcatcgacgccaagaccacccagccccccagcatggattgcgccgaaggcagagccgccaacctgccctgcaaccacagcaccatcagcggcaacgagtacgtgtactggtacagacagatccacagccagggcccccagtacatcatccacggcctgaagaacaacgagacaaacgagatggccagcctgatcatcaccgaggacagaaagagcagcaccctgatcctgccccacgccaccctgagagacaccgccgtgtactactgc

SEQ ID NO: 41 - DNA, 315 bp, Homo sapiens
atgaagctcgtgaccagcatcaccgtgctgctgagcctgggcatcatgggcgacgccaagaccacccagcccaacagcatggaaagcaacgaagaggaacccgtgcatctgccctgcaaccacagcaccatcagcggcaccgactacatccactggtacagacagctgcccagccagggccccgagtacgtgatccacggcctgaccagcaacgtgaacaaccggatggcctccctggccattgccgaggacagaaagagcagcaccctgatcctgcaccgggccaccctgagagatgccgccgtgtactactgc

SEQ ID NO: 42 - DNA, 321 bp, Homo sapiens
atggtgctgaagttcagcgtgtccatcctgtggatccagctggcctgggtgtccacccagctgctggaacagagcccccagttcctgagcatccaggaaggcgagaacctgaccgtgtactgcaacagcagcagcgtgttcagcagcctgcagtggtacagacaggaacccggcgagggccccgtgctgctggtcacagtcgtgacaggcggcgaagtgaagaagctgaagcggctgaccttccagttcggcgacgcccggaaggacagctccctgcacattacagccgcccagcccggcgacaccggcctgtatctgtgc

SEQ ID NO: 43 - DNA, 348 bp, Homo sapiens
atggctatgctgctgggcgccagcgtgctgatcctgtggctgcagcccgactgggtcaacagccagcagaagaacgacgaccagcaggtcaaacagaacagccccagcctgagcgtgcaggaaggccggatcagcatcctgaactgcgactacaccaactctatgttcgactacttcctgtggtacaagaagtaccccgccgagggccccaccttcctgatcagcatcagcagcatcaaggacaagaacgaggacggccggttcaccgtgtttctgaacaagagcgccaagcacctgagcctgcatatcgtgcccagccagcccggcgacagcgccgtgtacttttgc

SEQ ID NO: 44 - DNA, 327 bp, Homo sapiens
atggaaaccctgctgaaggtgctgagcggcaccctgctgtggcagctgacatgggtccgaagccagcagcccgtgcagagccctcaggccgtgatcctgagagaaggcgaggacgccgtgatcaactgcagcagcagcaaggccctgtacagcgtgcactggtacagacagaagcacggcgaggcccccgtgttcctgatgattctgctgaagggcggcgagcagaagggccacgagaagatcagcgccagcttcaacgagaagaagcagcagagcagcctgtacctgaccgccagccagctgagctacagcggcacctacttttgc

SEQ ID NO: 45 - DNA, 327 bp, Homo sapiens
atggagaccgtgctgcaggtgctgctgggcatcctgggcttccaggccgcctgggtgagcagccaggagctggagcagagcccccagagcctgatcgtgcaggagggcaagaacctgaccatcaactgcaccagcagcaagaccctgtacggcctgtactggtacaagcagaagtacggcgagggcctgatcttcctgatgatgctgcagaagggcggcgaggagaagagccacgagaagatcaccgccaagctggacgagaagaagcagcagagcagcctgcacatcaccgccagccagcccagccacgccggcatctacctgtgc

SEQ ID NO: 46 - DNA, 321 bp, Homo sapiens
atgctgctggaacatctgctgatcatcctgtggatgcagctgacctgggtgtccggccagcagctgaaccagagcccccagagcatgttcatccaggaaggcgaggacgtcagcatgaactgcaccagcagcagcatcttcaacacctggctgtggtacaagcaggaacccggcgagggccccgtgctgctgatcgccctgtataaggccggcgagctgaccagcaacggccggctgacagcccagttcggcattacccggaaggacagcttcctgaacatcagcgccagcatccccagcgacgtgggcatctacttttgc

SEQ ID NO: 47 - DNA, 330 bp, Homo sapiens
atgatgaagtgtccccaggccctgctggccatcttctggctgctgctgagctgggtgtccagcgaggacaaggtggtgcagagccccctgagcctggtggtgcacgagggcgataccgtgaccctgaactgcagctacgaagtgaccaacttccggtccctgctgtggtacaagcaggaaaagaaggcccccaccttcctgttcatgctgaccagcagcggcatcgagaagaagtccggcagactgagcagcatcctggacaagaaagagctgtccagcatcctgaacatcaccgccacccagaccggcgacagcgccatctacctgtgc

SEQ ID NO: 48 - DNA, 336 bp, Homo sapiens
atgaccagagtgtctctgctgtgggccgtggtggtgtccacctgtctggaaagcggcatggcccagaccgtgacccagagccagcccgagatgagcgtgcaggaagccgagacagtcaccctgagctgcacctacgacaccagcgagaacaactactacctgttctggtacaagcagccccccagccggcagatgatcctggtcatccggcaggaagcctacaagcagcagaacgccaccgagaacagattcagcgtgaacttccagaaggccgccaagagcttcagcctgaagatcagcgacagccagctgggcgacaccgccatgtacttttgc

SEQ ID NO: 49 - DNA, 336 bp, Homo sapiens
atggcctgtcctggatttctgtgggccctggtcatcagcacctgtctggaattcagcatggcccagaccgtgacccagagccagcccgagatgagcgtgcaggaagccgagacagtcaccctgagctgcacctacgacaccagcgagagcgactactacctgttctggtacaagcagccccccagccggcagatgatcctggtcatccggcaggaagcctacaagcagcagaacgccaccgagaacagattcagcgtgaacttccagaaggccgccaagagcttcagcctgaagatcagcgacagccagctgggcgacgccgccatgtacttttgc

SEQ ID NO: 50 - DNA, 321 bp, Homo sapiens
atgaagaaactgctggccatgatcctgtggctgcagctggaccggctgagcggcgagctgaaggtggaacagaaccccctgttcctgagcatgcaggaaggcaagaactacaccatctactgcaactacagcaccaccagcgaccggctgtactggtacagacaggaccccggcaagagcctggaaagcctgttcgtgctgctgagcaacggcgccgtgaagcaggaaggccggctgatggccagcctggacaccaaggccagactgagcaccctgcacatcacagccgccgtgcacgacctgagcgccacctacttttgc

SEQ ID NO: 51 - DNA, 306 bp, Homo sapiens
atgaacagcagcctggacttcctgatcctgattctgatgttcggcggcaccagcagcaacagcgtgaagcagaccggccagatcaccgtgtccgagggcgccagcgtgaccatgaactgcacctacaccagcaccggctaccccaccctgttttggtacgtggaataccccagcaagcccctgcagctcctgcagcgggaaaccatggaaaacagcaagaacttcggcggaggcaacatcaaggacaagaacagccccatcgtgaagtacagcgtccaggtgtccgacagcgccgtgtactactgc

SEQ ID NO: 52 - DNA, 328 bp, Homo sapiens
catggtcaagatccggcagttcctgctggccatcctgtggctgcagctgagctgtgtgtccgccgccaagaacgaggtggaacagagcccccagaacctgaccgcccaggaaggcgagttcatcaccatcaactgcagctacagcgtgggcatcagcgccctgcactggctgcagcagcatcccggcggaggcatcgtgtccctgttcatgctgagcagcggcaagaagaagcacggccggctgatcgccaccatcaacatccaggaaaagcacagcagcctgcacatcaccgccagccaccccagagacagcgccgtgtacatctgc

SEQ ID NO: 53 - DNA, 333 bp, Homo sapiens
atggatacctggctcgtgtgctgggccatcttcagcctgctgaaggccggcctgaccgagcccgaagtgacccagacccctagccaccaggtcacacagatgggccaggaagtgatcctgcgctgcgtgcccatcagcaaccacctgtacttctactggtacagacagatcctgggccagaaagtggaattcctggtgtccttctacaacaacgagatcagcgagaagtccgagatcttcgacgaccagttcagcgtggaacggcccgacggcagcaacttcaccctgaagatcagaagcaccaagctcgaggacagcgccatgtacttttgc

SEQ ID NO: 54 - DNA, 330 bp, Homo sapiens
atgggctgccgcctgctgtgctgcgtggtgttctgcctgctgcaggccggccccctggacaccgccgtgagccagacccccaagtacctggtgacccagatgggcaacgacaagagcatcaagtgcgagcagaacctgggccacgacaccatgtactggtacaagcaggacagcaagaagttcctgaagatcatgttcagctacaacaacaaggagctgatcatcaacgagaccgtgcccaaccgcttcagccccaagagccccgacaaggcccacctgaacctgcacatcaacagcctggagctgggcgacagcgccgtgtacttctgc

SEQ ID NO: 55 - DNA, 330 bp, Homo sapiens
atgggatgcagactgctgtgctgcgccgtgctgtgtctgctgggcgccgtgcccatcgacaccgaagtgacccagacccccaagcacctggtcatgggcatgaccaacaagaaaagcctgaagtgcgagcagcacatgggccaccgggccatgtactggtacaagcagaaggccaagaaaccccccgagctgatgttcgtgtacagctacgagaagctgagcatcaacgagagcgtgcccagccggttcagccccgagtgccccaatagcagcctgctgaacctgcatctgcacgccctgcagcccgaggacagcgccctgtatctgtgc

SEQ ID NO: 56 - DNA, 330 bp, Homo sapiens
atgggatgtagactgctgtgttgcgccgtgctgtgtctgctgggagccgtgcctatggaaaccggcgtgacccagacccccagacatctcgtgatgggcatgaccaacaagaaaagcctgaagtgcgagcagcacctgggccacaacgccatgtactggtacaagcagagcgccaagaaacccctggaactgatgttcgtgtacaacttcaaagagcagaccgagaacaacagcgtgcccagcagattcagccccgagtgccccaatagcagccacctgtttctgcatctgcacaccctgcagcccgaggacagcgccctgtatctgtgc

SEQ ID NO: 57 - DNA, 339 bp, Homo sapiens
atgggatgcagactgctgtgctgcgccgtgctgtgcctgctgggagctggcgagctggtgcccatggaaaccggcgtgacccagacccccagacacctggtcatgggcatgaccaacaagaaaagcctgaagtgcgagcagcacctgggccacaacgccatgtactggtacaagcagagcgccaagaaacccctggaactgatgttcgtgtacagcctggaagagagggtggaaaacaacagcgtgcccagccggttcagccccgagtgccccaatagcagccacctgtttctgcatctgcacaccctccagcccgaggacagcgccctgtatctgtgc

SEQ ID NO: 58 - DNA, 330 bp, Homo sapiens
atgggaagcagactgctgtgctgggtgctgctgtgcctgctgggagccggacctgtgaaggccggcgtgacccagacccccagatacctgatcaagaccagaggccagcaggtcacactgagctgcagccccatcagcggccacagaagcgtgtcctggtatcagcagaccccaggccagggcctgcagttcctgttcgagtacttcagcgagacacagcggaacaagggcaacttccccggcagattcagcggcagacagttcagcaacagccgcagcgagatgaacgtgtccaccctggaactgggcgacagcgccctgtatctgtgc

SEQ ID NO: 59 - DNA, 330 bp, Homo sapiens
atgggacctggactgctgtgttgggtgctgctgtgtctgctgggagccggcagtgtggaaaccggcgtgacacagagccccacccacctgatcaagaccagaggccagcaagtgaccctgcggtgcagctctcagagcggccacaataccgtgtcctggtatcagcaggccctgggccagggaccccagttcatcttccagtactacagagaggaagagaacggcagaggcaacttcccaccccggtttagcggcctgcagttccccaactacagctccgagctgaacgtgaacgccctggaactggacgacagcgccctgtacctgtgc

SEQ ID NO: 60 - DNA, 330 bp, Homo sapiens
atgggacctggactgctgtgttgggtgctgctgtgtctgctgggagccggacctgtggatgctggcgtgacacagagccccacccacctgatcaagaccagaggccagcaagtgaccctgcggtgcagccctatcagcggccacaagagcgtgtcctggtatcagcaggtgctgggccagggcccccagttcatcttccagtactacgagaaagaggaacggggcagaggcaacttccccgacagattcagcgccagacagttccccaactacagctccgagctgaacgtgaacgccctgctgctgggcgatagcgccctgtatctgtgc

SEQ ID NO: 61 - DNA, 330 bp, Homo sapiens
atgggacctggactgctgtgttgggccctgctgtgcctgctgggagccggactggtggatgccggcgtgacccagagccccacccacctgatcaagaccagaggccagcaggtcacactgcggtgcagccccaagagcggccacgacaccgtgtcctggtatcagcaggccctcggccagggaccccagttcatcttccagtactacgaggaagaggaacggcagcggggcaacttccccgacagattcagcggccaccagttccccaactacagcagcgagctgaacgtgaacgccctgctgctgggcgacagcgccctgtatctgtgc

SEQ ID NO: 62 - DNA, 330 bp, Homo sapiens
atgggacctagactgctgttttgggccctgctgtgcctgctgggcacaggacctgtggaagctggcgtgacccagagccctacccacctgatcaagaccagaggccagcaggccaccctgagatgcagccctatcagcggccacaccagcgtgtactggtatcagcaggccctgggactgggcctgcagttcctgctgtggtacgacgagggcgaggaacggaaccggggcaacttcccacccagattcagcggcagacagttccccaactacagcagcgagctgaacgtgaacgccctggaactggaagatagcgccctgtacctgtgc

SEQ ID NO: 63 - DNA, 330 bp, Homo sapiens
atgtctatcggcctgctgtgctgcgtggccttcagcctgctgtgggccagccctgtgaatgccggcgtgacccagacccccaagttccaggtgctgaaaaccggccagagcatgaccctgcagtgcgcccaggacatgaaccacaacagcatgtactggtacagacaggaccccggcatgggcctgcggctgatctactacagcgccagcgagggcaccaccgacaagggcgaggtgcccaacggctacaacgtgtcccggctgaacaagagagagttcagcctgagactggaaagcgccgctcccagccagaccagcgtgtacttctgc

SEQ ID NO: 64 - DNA, 330 bp, Homo sapiens
atgtctctgggcctgctgtgctgcggcgccttcagcctgctgtgggccggacctgtgaatgccggcgtgacccagacccccaagttccgggtgctgaaaaccggccagagcatgaccctgctgtgcgcccaggacatgaaccacgagtacatgtattggtacagacaggaccccggcatgggcctgcggctgatccactactctgtgggcgagggcaccaccgccaagggcgaagtgcccgacggctacaacgtgtcccggctgaagaagcagaacttcctgctgggcctggaaagcgccgctcccagccagaccagcgtgtacttctgc

SEQ ID NO: 65 - DNA, 330 bp, Homo sapiens
atgagaatcagactgctgtgctgcgtggccttcagcctgctgtgggccggacctgtgatcgccggaatcacccaggcccccaccagccagattctggccgctggcagacggatgaccctgcggtgtacccaggacatgcggcacaacgccatgtactggtacagacaggacctgggcctgggcctgcggctgatccactacagcaataccgccggcaccaccggcaagggcgaggtgccagatggctacagcgtgtcccgggccaacaccgacgacttcccactgacactggccagcgccgtgcccagccagaccagcgtgtacttctgc

SEQ ID NO: 66 - DNA, 330 bp, Homo sapiens
atgagcatcggcctgctgtgctgcgccgccctgagcctgctgtgggccggccccgtgaacgccggcgtgacccagacccccaagttccaggtgctgaagaccggccagagcatgaccctgcagtgcgcccaggacatgaaccacgagtacatgagctggtaccgccaggaccccggcatgggcctgcgcctgatccactacagcgtgggcgccggcatcaccgaccagggcgaggtgcccaacggctacaacgtgagccgcagcaccaccgaggacttccccctgcgcctgctgagcgccgcccccagccagaccagcgtgtacttctgc

SEQ ID NO: 67 - DNA, 330 bp, Homo sapiens
atgtctattagcctgctgtgctgcgccgccttccccctgctgtgggctggacctgtgaatgccggcgtgacccagacccccaagttccggatcctgaagatcggccagagcatgaccctgcagtgcacccaggacatgaaccacaactacatgtactggtacagacaggaccccggcatgggcctgaagctgatctactactctgtgggagccggcatcaccgacaagggcgaggtgcccaatggctacaacgtgtccagaagcaccaccgaggacttccctctgcggctggaactggccgctcctagccagaccagcgtgtacttctgc

SEQ ID NO: 68 - DNA, 327 bp, Homo sapiens
atgtctctgggactgctgtgctgcgccgccttctctctgctgtgggccggacctgtgaatgccggcgtgacccagacccccaagttccacatcctgaaaaccggccagagcatgaccctgcagtgcgcccaggacatgaaccacggctacatgagctggtacagacaggaccccggcatgggcctgcggctgatctactattctgccgccgctggcaccaccgacaaagaggtgccaaacggctacaacgtgtcccggctgaacaccgaggacttcccactgagactggtgtctgccgcccctagccagaccagcgtgtacctgtgc

SEQ ID NO: 69 - DNA, 330 bp, Homo sapiens
atgtctattggcctgctgtgctgcgtggccttcagtctgctgtgggagggccctgtgaatgccggcgtgacccagacccccaagttccacatcctgaaaaccggccagagcatgaccctgcagtgcgcccaggacatgaaccacggctacctgagctggtacagacaggaccctggcatgggcctgcggcggatccactattctgtggccgctggcatcaccgacaagggcgaagtgcccgacggctacaacgtgtccagaagcaacaccgaggacttcccactgcggctggaatctgccgcccctagccagaccagcgtgtacttctgc

SEQ ID NO: 70 - DNA, 333 bp, Homo sapiens
atgggcacccgcctgctgttctgggtggccttctgcctgctgggcgccgaccacaccggcgccggcgtgagccagagccccagcaacaaggtaactgagaagggcaaggacgtggagctgcgctgcgaccccatcagcggccacaccgccctgtactggtaccgccagagcctgggccagggcctggagttcctgatctacttccagggcaacagcgcccccgacaagagcggcctgcccagcgaccgcttcagcgccgagcgcaccggcggcagcgtgagcaccctgaccatccagcgcacccagcaggaggacagcgccgtgtacctgtgc

SEQ ID NO: 71 - DNA, 333 bp, Homo sapiens
atgggaaccagactgctgtgctgggccgctctgtgtctgctgggcgccgatcatacaggcgctggcgtgtcccagacccccagcaacaaagtgaccgagaagggcaaatacgtcgagctgagatgcgaccccatcagcggccacaccgccctgtactggtacagacagagcctgggccagggccccgagttcctgatctattttcagggcaccggcgctgcagacgacagcggcctgcccaacgacagattcttcgccgtgagacccgagggcagcgtgtccaccctgaagatccagcggaccgagaggggcgacagcgccgtgtatctgtgc

SEQ ID NO: 72 - DNA, 333 bp, Homo sapiens
atgggaacaagactgctgtgctgggtggtgctgggcttcctgggcacagatcatacaggcgctggcgtgtcccagagccccagatacaaggtggccaagaggggcagagatgtggccctgagatgcgacagcatcagcggccacgtgaccctgtactggtacagacagacactgggccagggcagcgaggtgctgacatacagccagagcgacgcccagcgggacaagagcggcagacctagcggcagattttccgccgagaggcccgagagaagcgtgtccaccctgaagatccagcggaccgagcagggcgatagcgccgtgtatctgtgc

SEQ ID NO: 73 - DNA, 333 bp, Homo sapiens
atgggaacaagcctgctgtgctgggtggtgctgggcttcctgggcaccgatcacacaggcgctggcgtgtcccagagcccccggtacaaagtgaccaagcggggccaggacgtggccctgagatgcgaccctatcagcggccatgtgtccctgtactggtacagacaggcactgggccagggacccgagttcctgacctacttcaactacgaggcccagcaggacaagagcggcctgcccaacgacagattcagcgccgagaggcccgagggcagcatcagcaccctgaccatccagcggaccgagcagcgggacagcgccatgtacagatgc

SEQ ID NO: 74 - DNA, 333 bp, Homo sapiens
atgggaacatctctgctgtgctgggtggtgctgggcttcctgggcacagatcatacaggcgctggcgtgtcccagagcccccggtacaaagtgaccaagaggggccaggacgtgaccctgagatgcgaccctatcagcagccacgccaccctgtactggtatcagcaggccctgggacagggccccgagttcctgacctacttcaactacgaggcccagcccgacaagagcggcctgcccagcgatagattttccgccgaaagacccgagggcagcatcagcaccctgaccatccagagaaccgagcagcgggacagcgccatgtacagatgc

SEQ ID NO: 75 - DNA, 333 bp, Homo sapiens
atgggaacaagactgctgtgctgggtggtgctgggcttcctgggcacagatcatacaggcgctggcgtgtcccagagccccagatacaaggtggccaagaggggccaggacgtggccctgagatgcgatcctatcagcggccacgtgtccctgttctggtatcagcaggccctgggccagggacccgagttcctgacctacttccagaacgaggcccagctggacaagagcggcctgcccagcgatagattcttcgccgaaagacccgagggcagcgtgtccaccctgaagatccagagaacccagcaggaagatagcgccgtgtacctgtgc

SEQ ID NO: 76 - DNA, 333 bp, Homo sapiens
atgggaacaagcctgctgtgctggatggccctgtgcctgctgggagccgaccacgccgatacaggcgtgtcccagaacccccggcacaagatcaccaagcggggccagaacgtgaccttcagatgcgaccccatcagcgagcacaaccggctgtactggtacagacagaccctgggccagggccccgagttcctgacctacttccagaacgaggcccagctggaaaagagccggctgctgagcgacagattcagcgccgagaggcccaagggcagcttcagcaccctggaaatccagcggaccgagcagggcgacagcgccatgtatctgtgc

SEQ ID NO: 77 - DNA, 330 bp, Homo sapiens
atgggcttccgcctgctgtgctgcgtggccttctgcctgctgggcgccggccccgtggacagcggcgtgacccagacccccaagcacctgatcaccgccaccggccagcgcgtgaccctgcgctgcagcccccgcagcggcgacctgagcgtgtactggtaccagcagagcctggaccagggcctgcagttcctgatccagtactacaacggcgaggagcgcgccaagggcaacatcctggagcgcttcagcgcccagcagttccccgacctgcacagcgagctgaacctgagcagcctggagctgggcgacagcgccctgtacttctgc

SEQ ID NO: 78 - DNA, 330 bp, Homo sapiens
atgggcaccagactgttcttctacgtggccctgtgcctgctgtgggccggacacagagatgccgagatcacccagagccccagacacaagatcaccgagacaggccggcaggtcacactggcctgccaccagacctggaaccacaacaacatgttctggtacagacaggacctgggccacggcctgcggctgatccactacagctacggcgtgcaggacaccaacaagggcgaggtgtccgacggctacagcgtgtccagaagcaacaccgaggacctgcccctgaccctggaaagcgccgccagcagccagaccagcgtgtacttctgc

SEQ ID NO: 79 - DNA, 330 bp, Homo sapiens
atgggaaccagactgttcttctacgtggccctgtgcctgctgtgggccggacatagagatgccggaatcacccagagcccccggtacaagatcaccgagacaggcagacaagtgaccctgatgtgccaccagacctggtcccacagctacatgttctggtacagacaggacctgggccacggcctgcggctgatctactattctgccgccgctgacatcaccgacaagggcgaagtgcccgacggctacgtggtgtccagaagcaagaccgagaacttcccactgaccctggaaagcgccacccggtcccagaccagcgtgtacttttgc

SEQ ID NO: 80 - DNA, 330 bp, Homo sapiens
atgggcaccagactgttcttctacgtggccctgtgcctgctgtggaccggccacatggacgccggcatcacccagagccccagacacaaagtcaccgagacaggcacccccgtgaccctgagatgccaccagaccgagaaccaccgctacatgtactggtacagacaggaccccggccacggcctgcggctgatccactacagctacggcgtgaaggacaccgacaagggcgaggtgtccgacggctacagcgtgtccagaagcaagaccgaggacttcctgctgaccctggaaagcgccaccagcagccagaccagcgtgtacttctgc

SEQ ID NO: 81 - DNA, 333 bp, Homo sapiens
atgagcaccagactgctgtgctggatggccctgtgcctgctgggcgccgagctgtctgaagccgaggtggcccagagcccccggtacaagatcaccgagaagtcccaggccgtggccttttggtgcgaccccatcagcggccacgccaccctgtactggtacagacagatcctgggccagggccccgaactgctggtgcagttccaggacgagagcgtggtggacgacagccagctgcccaaggacagattcagcgccgagcggctgaagggcgtggacagcaccctgaagatccagcccgccgagctgggcgacagcgccatgtatctgtgc

SEQ ID NO: 82 - DNA, 333 bp, Homo sapiens
atgggaaccagactgctgtgctgggccgctctgtgtctgctgggcgccgaactgacagaggctggcgtggcacagagcccccggtacaagatcatcgagaagcggcagagcgtggccttctggtgcaaccccatcagcggccacgccaccctgtactggtatcagcagatcctgggccagggccccaagctgctgatccagttccagaacaacggcgtggtggacgacagccagctgcccaaggacagattcagcgccgagcggctgaagggcgtggacagcaccctgaagatccagcccgccaagctggaagatagcgccgtgtatctgtgc

SEQ ID NO: 83 - DNA, 333 bp, Homo sapiens
atgggaacaagactgctgtgctgggtggccttctgcctgctggtggaagaactgatcgaggccggcgtggtgcagagcccccggtacaagatcatcgagaagaaacagcccgtggccttttggtgcaaccccatcagcggccacaacaccctgtactggtatctgcagaacctgggccagggccccgagctgctgatcagatacgagaacgaggaagccgtggacgacagccagctgcccaaggacagattcagcgccgagagactgaagggcgtggacagcaccctgaagatccagcctgccgagctgggcgatagcgccgtgtatctgtgc

SEQ ID NO: 84 - DNA, 333 bp, Homo sapiens
atggacagctggaccttctgctgcgtgagcctgtgcatcctggtggccaagcacaccgacgccggcgtgatccagagcccccgccacgaggttactgagatgggccaggaggtgactctgcgctgcaagcccatcagcggccacaacagcctgttctggtaccgccagaccatgatgcgcggcctggagctgctgatctacttcaacaacaacgtgcccatcgacgacagcggcatgcccgaggaccgcttcagcgccaagatgcccaacgccagcttcagcaccctgaagatccagcccagcgagccccgcgacagcgccgtgtacttctgc

SEQ ID NO: 85 - DNA, 333 bp, Homo sapiens
atgggatcttggacactgtgctgcgtgtccctgtgcatcctggtggccaagcacacagatgccggcgtgatccagagccccagacatgaagtgaccgagatgggccaggaagtgaccctgcgctgcaagcctatcagcggccacgactacctgttctggtacagacagaccatgatgcggggcctggaactgctgatctacttcaacaacaacgtgcccatcgacgacagcggcatgcccgaggatagattcagcgccaagatgcccaacgccagcttcagcaccctgaagatccagcccagcgagcccagagacagcgccgtgtacttttgc

SEQ ID NO: 86 - DNA, 333 bp, Homo sapiens
atggctaccagactgctgtgctgcgtggtgctgtgcctgctgggcgaggaactgatcgacgccagagtgacccagacccccagacacaaagtcaccgagatgggccaggaagtgaccatgcggtgccagcccatcctgggccacaacaccgtgttctggtacagacagaccatgatgcagggcctggaactgctggcctacttccggaacagagcccccctggacgacagcggcatgcccaaggacagattcagcgccgagatgcccgacgccaccctggccacactgaagatccagcccagcgagcccagagacagcgccgtgtacttctgc

SEQ ID NO: 87 - DNA, 360 bp, Homo sapiens
atgctgtctcccgacctgcctgacagcgcctggaacacccggctgctgtgccatgtgatgctgtgcctgctgggcgccgtgtctgtggccgctggcgtgatccagagccccagacacctgatcaaagagaagagagagacagccaccctgaagtgctaccccatcccccggcacgacaccgtgtactggtatcagcagggccctggacaggacccccagttcctgatcagcttctacgagaagatgcagagcgacaagggcagcatccccgacagattcagcgcccagcagttcagcgactaccacagcgagctgaacatgagcagcctggaactgggcgacagcgccctgtacttctgc

SEQ ID NO: 88 - DNA, 333 bp, Homo sapiens
atggtgagccgcctgctgagcctggtgagcctgtgcctgctgggcgccaagcacatcgaggccggcgtgacccagttccccagccacagcgtgatcgagaagggccagaccgtgaccctgcgctgcgaccccatcagcggccacgacaacctgtactggtatcgccgcgtgatgggcaaggagatcaagttcctgctgcacttcgtgaaggagagcaagcaggacgagagcggcatgcccaacaaccgcttcctggccgagcgcaccggcggcacctacagcaccctgaaggtgcagcccgccgagctggaggacagcggcgtgtacttctgc

SEQ ID NO: 89 - DNA, 330 bp, Homo sapiens
atgggacctggactgctgcactggatggccctgtgcctgctgggcacaggacacggcgacgccatggtcatccagaaccccagataccaggtcacacagttcggcaagcccgtgaccctgagctgcagccagaccctgaaccataacgtgatgtactggtatcagcagaagtccagccaggcccccaagctgctgttccactactacgacaaggacttcaacaacgaggccgacacccccgacaacttccagagcagacggcccaataccagcttctgcttcctggacatcagaagccctggcctgggcgacaccgccatgtatctgtgc

SEQ ID NO: 90 - DNA, 333 bp, Homo sapiens
atgagccccatcttcacctgtatcaccatcctgtgcctgctggccgctggcagccctggcgaagaagtggcccagacccccaagcacctcgtcagaggcgagggccagaaggccaagctgtactgcgcccccatcaagggccacagctacgtgttctggtatcagcaggtcctgaagaacgagttcaagttcctgatcagcttccagaacgagaacgtgttcgacgagacaggcatgcccaaagagcggttcagcgccaagtgcctgcccaacagcccctgcagcctggaaatccaggccaccaagctggaagatagcgccgtgtacttctgc

SEQ ID NO: 91 - DNA, 333 bp, Homo sapiens
atggataccagagtgctgtgctgcgccgtgatctgcctgctgggagccggcctgtctaacgccggcgtgatgcagaacccccggcatctcgtgcggcggagaggccaggaagcccggctgcgctgtagccccatgaagggccacagccatgtgtactggtacagacagctgcccgaagagggcctgaagttcatggtgtacctgcagaaagagaacatcatcgacgagagcggcatgcccaaagagcggttcagcgccgagttccccaaagagggccccagcatcctgagaatccagcaggtcgtgcggggcgatagcgccgcctacttctgc

SEQ ID NO: 92 - DNA, 330 bp, Homo sapiens
atgagcaatcaggtgctgtgctgcgtggtgctgtgtttcctgggcgccaacaccgtggacggcggcatcacccagagccccaagtacctgttccggaaagagggccagaacgtcaccctgagctgcgagcagaacctgaaccacgacgccatgtactggtacagacaggaccccggccagggcctgcggctgatctactacagccagatcgtgaacgacttccagaagggcgacattgccgagggctacagcgtgtccagagagaagaaagagtccttcccactgaccgtgaccagcgcccagaagaaccccaccgccttctacctgtgc

SEQ ID NO: 93 - DNA, 357 bp, Homo sapiens
atgctgctgctgctgctcctcctgggccctggcatcagcctgctgctgcctggatctctggccggctctggactgggcgccgtggtgtctcagcaccccagctgggtcatctgcaagagcggcaccagcgtgaagatcgagtgcagaagcctggacttccaggccaccaccatgttctggtacagacagttccccaagcagagcctgatgctgatggccaccagcaacgagggcagcaaggccacctacgagcagggcgtggaaaaggacaagttcctgatcaaccacgccagcctgaccctgagcaccctgaccgtgacaagcgcccaccccgaggacagcagcttctacatctgc

SEQ ID NO: 94 - DNA, 330 bp, Homo sapiens
atggctagcctgctgttcttctgcggcgccttctacctgctgggcaccggcagcatggacgccgacgtgacccagaccccccggaaccggatcaccaagaccggcaagcggatcatgctggaatgcagccagaccaagggccacgaccggatgtactggtacagacaggaccccggcctgggcctgcggctgatctactacagcttcgacgtgaaggacatcaacaagggcgagatcagcgacggctacagcgtgtccagacaggcccaggctaagttcagcctgagcctggaaagcgccatccccaaccagaccgccctgtacttctgc

SEQ ID NO: 95 - DNA, 330 bp, Homo sapiens
atgaccatcagactgctgtgctatatgggcttctacttcctgggagccggcctgatggaagccgacatctaccagacccccagatacctggtcatcggcaccggcaagaaaatcaccctggaatgcagccagaccatgggccacgacaagatgtactggtatcagcaggaccccggcatggaactgcacctgatccactacagctacggcgtgaacagcaccgagaagggcgacctgagcagcgagagcaccgtgtcccggatccggaccgagcacttcccactgaccctggaaagcgccaggcccagccacaccagccagtacctgtgc

SEQ ID NO: 96 - DNA, 330 bp, Homo sapiens
atgggacctcagctgctgggctacgtggtgctgtgcctgctgggagccggacccctggaagcccaggtcacacagaaccccagatacctgatcaccgtgaccggcaagaaactgaccgtgacctgcagccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgc

SEQ ID NO: 97 - DNA, 330 bp, Homo sapiens
atgggaatcagactgctgtgccgggtggccttctgcttcctggccgtgggactggtggacgtgaaagtgacccagagcagcagatacctggtcaagcggaccggcgagaaggtgttcctggaatgcgtgcaggacatggaccacgagaatatgttctggtacagacaggaccccggcctgggcctgcggctgatctacttcagctacgacgtgaagatgaaggaaaagggcgacatccccgagggctacagcgtgtccagagagaagaaagagcggttcagcctgatcctggaaagcgccagcaccaaccagaccagcatgtatctgtgc

SEQ ID NO: 98 - DNA, 324 bp, Homo sapiens
atgctgagcctgctgctgctgctgctgggcctgggcagcgtgttcagcgccgtgatcagccagaagcccagccgcgacatctgccagcgcggcaccagcctgaccatccagtgccaggtggacagccaggtgaccatgatgttctggtaccgccagcagcccggccagagcctgaccctgatcgccaccgccaaccagggcagcgaggccacctacgagagcggcttcgtgatcgacaagttccccatcagccgccccaacctgaccttcagcaccctgaccgtgagcaacatgagccccgaggacagcagcatctacctgtgc

SEQ ID NO: 99 - DNA, 324 bp, Homo sapiens
atgctgtgttctctgctggccctgctgctgggcaccttcttcggagtgcggagccagaccatccaccagtggcctgccaccctggtgcagcctgtgggcagccctctgagcctggaatgcaccgtggaaggcaccagcaaccccaacctgtactggtacagacaggccgctggcagaggcctgcagctgctgttctacagcgtgggcatcggccagatcagcagcgaggtgccccagaacctgagcgccagcagaccccaggaccggcagttcatcctgagcagcaagaagctgctgctgagcgacagcggcttctacctgtgc

SEQ ID NO: 100 - protein, 88 aa, Homo sapiens
GQSLEQPSEVTAVEGAIVQINCTYQTSGFYGLSWYQQHDGGAPTFLSYNALDGLEETGRFSSFLSRSDSYGYLLLQELQMKDSASYFC

SEQ ID NO: 101 - protein, 88 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLC

SEQ ID NO: 102 - protein, 84 aa, Homo sapiens
KDQVFQPSTVASSEGAVVEIFCNHSVSNAYNFFWYLHFPGCAPRLLVKGSKPSQQGRYNMTYERFSSSLLILQVREADAAVYYC

SEQ ID NO: 103 - protein, 91 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFC

SEQ ID NO: 104 - protein, 88 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYC

SEQ ID NO: 105 - protein, 89 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFC

SEQ ID NO: 106 - protein, 90 aa, Homo sapiens
SQKIEQNSEALNIQEGKTATLTCNYTNYSPAYLQWYRQDPGRGPVFLLLIRENEKEKRKERLKVTFDTTLKQSLFHITASQPADSATYLC

SEQ ID NO: 107 - protein, 88 aa, Homo sapiens
ENQVEHSPHFLGPQQGDVASMSCTYSVSRFNNLQWYRQNTGMGPKHLLSMYSAGYEKQKGRLNATLLKNGSSLYITAVQPEDSATYFC

SEQ ID NO: 108 - protein, 91 aa, Homo sapiens
AQSVSQHNHHVILSEAASLELGCNYSYGGTVNLFWYVQYPGQHLQLLLKYFSGDPLVKGIKGFEAEFIKSKFSFNLRKPSVQWSDTAEYFC

SEQ ID NO: 109 - protein, 91 aa, Homo sapiens
AQSVTQLDSHVSVSEGTPVLLRCNYSSSYSPSLFWYVQHPNKGLQLLLKYTSAATLVKGINGFEAEFKKSETSFHLTKPSAHMSDAAEYFC

SEQ ID NO: 110 - protein, 91 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFC

SEQ ID NO: 111 - protein, 91 aa, Homo sapiens
AQSVTQLGSHVSVSEGALVLLRCNYSSSVPPYLFWYVQYPNQGLQLLLKYTSAATLVKGINGFEAEFKKSETSFHLTKPSAHMSDAAEYFC

SEQ ID NO: 112 - protein, 91 aa, Homo sapiens
AQSVTQLDSQVPVFEEAPVELRCNYSSSVSVYLFWYVQYPNQGLQLLLKYLSGSTLVESINGFEAEFNKSQTSFHLRKPSVHISDTAEYFC

SEQ ID NO: 113 - protein, 90 aa, Homo sapiens
GDSVVQTEGQVLPSEGDSLIVNCSYETTQYPSLFWYVQYPGEGPQLHLKAMKANDKGRNKGFEAMYRKETTSFHLEKDSVQESDSAVYFC

SEQ ID NO: 114 - protein, 90 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFC

SEQ ID NO: 115 - protein, 90 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYIC

SEQ ID NO: 116 - protein, 88 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLC

SEQ ID NO: 117 - protein, 89 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLC

SEQ ID NO: 118 - protein, 89 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLC

SEQ ID NO: 119 - protein, 90 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFC

SEQ ID NO: 120 - protein, 90 aa, Homo sapiens
GESVGLHLPTLSVQEGDNSIINCAYSNSASDYFIWYKQESGKGPQFIIDIRSNMDKRQGQRVTVLLNKTVKHLSLQIAATQPGDSAVYFC

SEQ ID NO: 121 - protein, 92 aa, Homo sapiens
AQKITQTQPGMFVQEKEAVTLDCTYDTSDPSYGLFWYKQPSSGEMIFLIYQGSYDQQNATEGRYSLNFQKARKSANLVISASQLGDSAMYFC

SEQ ID NO: 122 - protein, 87 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYC

SEQ ID NO: 123 - protein, 89 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFC

SEQ ID NO: 124 - protein, 89 aa, Homo sapiens
GDSVTQTEGPVTLPERAALTLNCTYQSSYSTFLFWYVQYLNKEPELLLKSSENQETDSRGFQASPIKSDSSFHLEKPSVQLSDSAVYYC

SEQ ID NO: 125 - protein, 92 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFC

SEQ ID NO: 126 - protein, 88 aa, Homo sapiens
EDQVTQSPEALRLQEGESSSLNCSYTVSGLRGLFWYRQDPGKGPEFLFTLYSAGEEKEKERLKATLTKKESFLHITAPKPEDSATYLC

SEQ ID NO: 127 - protein, 90 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLC

SEQ ID NO: 128 - protein, 87 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFC

SEQ ID NO: 129 - protein, 90 aa, Homo sapiens
QQQVKQSPQSLIVQKGGISIINCAYENTAFDYFPWYQQFPGKGPALLIAIRPDVSEKKEGRFTISFNKSAKQFSLHIMDSQPGDSATYFC

SEQ ID NO: 130 - protein, 90 aa, Homo sapiens
ILNVEQSPQSLHVQEGDSTNFTCSFPSSNFYALHWYRWETAKSPEALFVMTLNGDEKKKGRISATLNTKEGYSYLYIKGSQPEDSATYLC

SEQ ID NO: 131 - protein, 89 aa, Homo sapiens
GQQVMQIPQYQHVQEGEDFTTYCNSSTTLSNIQWYKQRPGGHPVFLIQLVKSGEVKKQKRLTFQFGEAKKNSSLHITATQTTDVGTYFC

SEQ ID NO: 132 - protein, 88 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYC

SEQ ID NO: 133 - protein, 88 aa, Homo sapiens
DAKTTQPNSMESNEEEPVHLPCNHSTISGTDYIHWYRQLPSQGPEYVIHGLTSNVNNRMASLAIAEDRKSSTLILHRATLRDAAVYYC

SEQ ID NO: 134 - protein, 89 aa, Homo sapiens
TQLLEQSPQFLSIQEGENLTVYCNSSSVFSSLQWYRQEPGEGPVLLVTVVTGGEVKKLKRLTFQFGDARKDSSLHITAAQPGDTGLYLC

SEQ ID NO: 135 - protein, 90 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFC

SEQ ID NO: 136 - protein, 88 aa, Homo sapiens
QQPVQSPQAVILREGEDAVINCSSSKALYSVHWYRQKHGEAPVFLMILLKGGEQKGHEKISASFNEKKQQSSLYLTASQLSYSGTYFC

SEQ ID NO: 137 - protein, 89 aa, Homo sapiens
SQELEQSPQSLIVQEGKNLTINCTSSKTLYGLYWYKQKYGEGLIFLMMLQKGGEEKSHEKITAKLDEKKQQSSLHITASQPSHAGIYLC

SEQ ID NO: 138 - protein, 89 aa, Homo sapiens
GQQLNQSPQSMFIQEGEDVSMNCTSSSIFNTWLWYKQEPGEGPVLLIALYKAGELTSNGRLTAQFGITRKDSFLNISASIPSDVGIYFC

SEQ ID NO: 139 - protein, 89 aa, Homo sapiens
EDKVVQSPLSLVVHEGDTVTLNCSYEVTNFRSLLWYKQEKKAPTFLFMLTSSGIEKKSGRLSSILDKKELSSILNITATQTGDSAIYLC

SEQ ID NO: 140 - protein, 92 aa, Homo sapiens
AQTVTQSQPEMSVQEAETVTLSCTYDTSENNYYLFWYKQPPSRQMILVIRQEAYKQQNATENRFSVNFQKAAKSFSLKISDSQLGDTAMYFC

SEQ ID NO: 141 - protein, 92 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFC

SEQ ID NO: 142 - protein, 89 aa, Homo sapiens
ELKVEQNPLFLSMQEGKNYTIYCNYSTTSDRLYWYRQDPGKSLESLFVLLSNGAVKQEGRLMASLDTKARLSTLHITAAVHDLSATYFC

SEQ ID NO: 143 - protein, 84 aa, Homo sapiens
SNSVKQTGQITVSEGASVTMNCTYTSTGYPTLFWYVEYPSKPLQLLQRETMENSKNFGGGNIKDKNSPIVKYSVQVSDSAVYYC

SEQ ID NO: 144 - protein, 87 aa, Homo sapiens
KNEVEQSPQNLTAQEGEFITINCSYSVGISALHWLQQHPGGGIVSLFMLSSGKKKHGRLIATINIQEKHSSLHITASHPRDSAVYIC

SEQ ID NO: 145 - protein, 92 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFC

SEQ ID NO: 146 - protein, 91 aa, Homo sapiens
DTAVSQTPKYLVTQMGNDKSIKCEQNLGHDTMYWYKQDSKKFLKIMFSYNNKELIINETVPNRFSPKSPDKAHLNLHINSLELGDSAVYFC

SEQ ID NO: 147 - protein, 91 aa, Homo sapiens
DTEVTQTPKHLVMGMTNKKSLKCEQHMGHRAMYWYKQKAKKPPELMFVYSYEKLSINESVPSRFSPECPNSSLLNLHLHALQPEDSALYLC

SEQ ID NO: 148 - protein, 91 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLC

SEQ ID NO: 149 - protein, 91 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLC

SEQ ID NO: 150 - protein, 91 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLC

SEQ ID NO: 151 - protein, 91 aa, Homo sapiens
ETGVTQSPTHLIKTRGQQVTLRCSSQSGHNTVSWYQQALGQGPQFIFQYYREEENGRGNFPPRFSGLQFPNYSSELNVNALELDDSALYLC

SEQ ID NO: 152 - protein, 91 aa, Homo sapiens
DAGVTQSPTHLIKTRGQQVTLRCSPISGHKSVSWYQQVLGQGPQFIFQYYEKEERGRGNFPDRFSARQFPNYSSELNVNALLLGDSALYLC

SEQ ID NO: 153 - protein, 91 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLC

SEQ ID NO: 154 - protein, 91 aa, Homo sapiens
EAGVTQSPTHLIKTRGQQATLRCSPISGHTSVYWYQQALGLGLQFLLWYDEGEERNRGNFPPRFSGRQFPNYSSELNVNALELEDSALYLC

SEQ ID NO: 155 - protein, 91 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFC

SEQ ID NO: 156 - protein, 91 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFC

SEQ ID NO: 157 - protein, 91 aa, Homo sapiens
IAGITQAPTSQILAAGRRMTLRCTQDMRHNAMYWYRQDLGLGLRLIHYSNTAGTTGKGEVPDGYSVSRANTDDFPLTLASAVPSQTSVYFC

SEQ ID NO: 158 - protein, 91 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFC

SEQ ID NO: 159 - protein, 91 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFC

SEQ ID NO: 160 - protein, 90 aa, Homo sapiens
NAGVTQTPKFHILKTGQSMTLQCAQDMNHGYMSWYRQDPGMGLRLIYYSAAAGTTDKEVPNGYNVSRLNTEDFPLRLVSAAPSQTSVYLC

SEQ ID NO: 161 - protein, 91 aa, Homo sapiens
NAGVTQTPKFHILKTGQSMTLQCAQDMNHGYLSWYRQDPGMGLRRIHYSVAAGITDKGEVPDGYNVSRSNTEDFPLRLESAAPSQTSVYFC

SEQ ID NO: 162 - protein, 92 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLC

SEQ ID NO: 163 - protein, 92 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLC

SEQ ID NO: 164 - protein, 92 aa, Homo sapiens
GAGVSQSPRYKVAKRGRDVALRCDSISGHVTLYWYRQTLGQGSEVLTYSQSDAQRDKSGRPSGRFSAERPERSVSTLKIQRTEQGDSAVYLC

SEQ ID NO: 165 - protein, 92 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRC

SEQ ID NO: 166 - protein, 92 aa, Homo sapiens
GAGVSQSPRYKVTKRGQDVTLRCDPISSHATLYWYQQALGQGPEFLTYFNYEAQPDKSGLPSDRFSAERPEGSISTLTIQRTEQRDSAMYRC

SEQ ID NO: 167 - protein, 92 aa, Homo sapiens
GAGVSQSPRYKVAKRGQDVALRCDPISGHVSLFWYQQALGQGPEFLTYFQNEAQLDKSGLPSDRFFAERPEGSVSTLKIQRTQQEDSAVYLC

SEQ ID NO: 168 - protein, 92 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLC

SEQ ID NO: 169 - protein, 91 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFC

SEQ ID NO: 170 - protein, 91 aa, Homo sapiens
DAEITQSPRHKITETGRQVTLACHQTWNHNNMFWYRQDLGHGLRLIHYSYGVQDTNKGEVSDGYSVSRSNTEDLPLTLESAASSQTSVYFC

SEQ ID NO: 171 - protein, 91 aa, Homo sapiens
DAGITQSPRYKITETGRQVTLMCHQTWSHSYMFWYRQDLGHGLRLIYYSAAADITDKGEVPDGYVVSRSKTENFPLTLESATRSQTSVYFC

SEQ ID NO: 172 - protein, 91 aa, Homo sapiens
DAGITQSPRHKVTETGTPVTLRCHQTENHRYMYWYRQDPGHGLRLIHYSYGVKDTDKGEVSDGYSVSRSKTEDFLLTLESATSSQTSVYFC

SEQ ID NO: 173 - protein, 92 aa, Homo sapiens
EAEVAQSPRYKITEKSQAVAFWCDPISGHATLYWYRQILGQGPELLVQFQDESVVDDSQLPKDRFSAERLKGVDSTLKIQPAELGDSAMYLC

SEQ ID NO: 174 - protein, 92 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLC

SEQ ID NO: 175 - protein, 92 aa, Homo sapiens
EAGVVQSPRYKIIEKKQPVAFWCNPISGHNTLYWYLQNLGQGPELLIRYENEEAVDDSQLPKDRFSAERLKGVDSTLKIQPAELGDSAVYLC

SEQ ID NO: 176 - protein, 92 aa, Homo sapiens
DAGVIQSPRHEVTEMGQEVTLRCKPISGHNSLFWYRQTMMRGLELLIYFNNNVPIDDSGMPEDRFSAKMPNASFSTLKIQPSEPRDSAVYFC

SEQ ID NO: 177 - protein, 92 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFC

SEQ ID NO: 178 - protein, 92 aa, Homo sapiens
DARVTQTPRHKVTEMGQEVTMRCQPILGHNTVFWYRQTMMQGLELLAYFRNRAPLDDSGMPKDRFSAEMPDATLATLKIQPSEPRDSAVYFC

SEQ ID NO: 179 - protein, 91 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFC

SEQ ID NO: 180 - protein, 92 aa, Homo sapiens
EAGVTQFPSHSVIEKGQTVTLRCDPISGHDNLYWYRRVMGKEIKFLLHFVKESKQDESGMPNNRFLAERTGGTYSTLKVQPAELEDSGVYFC

SEQ ID NO: 181 - protein, 91 aa, Homo sapiens
DAMVIQNPRYQVTQFGKPVTLSCSQTLNHNVMYWYQQKSSQAPKLLFHYYDKDFNNEADTPDNFQSRRPNTSFCFLDIRSPGLGDTAMYLC

SEQ ID NO: 182 - protein, 92 aa, Homo sapiens
GEEVAQTPKHLVRGEGQKAKLYCAPIKGHSYVFWYQQVLKNEFKFLISFQNENVFDETGMPKERFSAKCLPNSPCSLEIQATKLEDSAVYFC

SEQ ID NO: 183 - protein, 92 aa, Homo sapiens
NAGVMQNPRHLVRRRGQEARLRCSPMKGHSHVYWYRQLPEEGLKFMVYLQKENIIDESGMPKERFSAEFPKEGPSILRIQQVVRGDSAAYFC

SEQ ID NO: 184 - protein, 91 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLC

SEQ ID NO: 185 - protein, 94 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYIC

SEQ ID NO: 186 - protein, 91 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFC

SEQ ID NO: 187 - protein, 91 aa, Homo sapiens
EADIYQTPRYLVIGTGKKITLECSQTMGHDKMYWYQQDPGMELHLIHYSYGVNSTEKGDLSSESTVSRIRTEHFPLTLESARPSHTSQYLC

SEQ ID NO: 188 - protein, 91 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFC

SEQ ID NO: 189 - protein, 91 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLC

SEQ ID NO: 190 - protein, 93 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLC

SEQ ID NO: 191 - protein, 91 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLC

SEQ ID NO: 192 - DNA, 27 bp, Mus musculus
gcagccagcgacaactaccagctgatc

SEQ ID NO: 193 - DNA, 30 bp, Mus musculus
gcaagcagccgcgccaactacgagcagtac

SEQ ID NO: 194 - DNA, 15 bp, Artificial Sequence
  synthetic
gcacacacggcaata

SEQ ID NO: 195 - DNA, 15 bp, Artificial Sequence
  synthetic
gcacacacggcaata

SEQ ID NO: 196 - DNA, 2703 bp, Artificial Sequence
  synthetic
tgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgcatgcctgcaggtcgactctagaggatccaccggtcgccaccggtaccgagctcgaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcg

SEQ ID NO: 197 - DNA, 3114 bp, Artificial Sequence
  synthetic
tgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgcatgcctgcaggtcgactctagaggatccaccggtcgccaccatccagaaccccgagcccgccgtgtaccagctgaaggaccccagaagccaggacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctgaggtaccgagctcgaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcg

SEQ ID NO: 198 - DNA, 3225 bp, Artificial Sequence
  synthetic
tgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgcatgcctgcaggtcgactctagaggatccaccggtcgccaccgaagatctgaggaacgtgaccccccccaaggtgaccctgttcgagcccagcaaggccgagatcgccaacaagcagaaagccaccctggtctgcctggccaggggcttcttccccgaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagctgaggtaccgagctcgaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcg

SEQ ID NO: 199 - DNA, 3551 bp, Artificial Sequence
  synthetic
tgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgcatgcctgctggtcgactctagaggatccaccggtcgccaccgcggccgccatggtgagaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagcggcagcggcgccaccaacttcagcctgctgaagcaggccggcgacgtggaggaaaaccctgggcctgcaggatgctgacgacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctgagaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcg

SEQ ID NO: 200 - DNA, 5477 bp, Artificial Sequence
  synthetic
tcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgcaccggtcgccaccggtaccgagctcgaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcaga

SEQ ID NO: 201 - DNA, 5888 bp, Artificial Sequence
  synthetic
tcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgcaccggtcgccaccatccagaaccccgagcccgccgtgtaccagctgaaggaccccagaagccaggacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctgaggtaccgagctcgaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcaga

SEQ ID NO: 202 - DNA, 5999 bp, Artificial Sequence
  synthetic
tcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgcaccggtcgccaccgaagatctgaggaacgtgaccccccccaaggtgaccctgttcgagcccagcaaggccgagatcgccaacaagcagaaagccaccctggtctgcctggccaggggcttcttccccgaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagctgaggtaccgagctcgaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcaga

SEQ ID NO: 203 - DNA, 6304 bp, Artificial Sequence
  synthetic
tcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgccatggtgagaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagcggcagcggcgccaccaacttcagcctgctgaagcaggccggcgacgtggaggaaaaccctgggcctgcaggatgctgacgacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctgagaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcaga

SEQ ID NO: 204 - DNA, 6266 bp, Artificial Sequence
  synthetic
tcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgcaccggtcgccagcatgtggggagcctttctgctgtacgtgtccatgaagatgggcggcacagccggccagagcctggaacagcctagcgaagtgaccgccgtggaaggcgccatcgtgcagatcaactgcacctaccagaccagcggcttctacggcctgagctggtatcagcagcacgacggcggagcccccaccttcctgagctacaacgccctggacggcctggaagagacaggccggttcagcagcttcctgagcagaagcgacagctacggctacctgctgctgcaggaactgcagatgaaggacagcgccagctacttctgcgcagccagcgacaactaccagctgatctggggcagcggcaccaagctgatcatcaagcccgacatccagaaccccgagcccgccgtgtaccagctgaaggaccccagaagccaggacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctgaggtaccgagctcgaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcaga

SEQ ID NO: 205 - DNA, 6392 bp, Artificial Sequence
  synthetic
tcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgcaccggtcgccaccatggatacctggctcgtgtgctgggccatcttcagcctgctgaaggccggcctgaccgagcccgaagtgacccagacccctagccaccaggtcacacagatgggccaggaagtgatcctgcgctgcgtgcccatcagcaaccacctgtacttctactggtacagacagatcctgggccagaaagtggaattcctggtgtccttctacaacaacgagatcagcgagaagtccgagatcttcgacgaccagttcagcgtggaacggcccgacggcagcaacttcaccctgaagatcagaagcaccaagctcgaggacagcgccatgtacttttgcgcaagcagccgcgccaactacgagcagtacttcggccccggcacccgcctgaccgtgctggaagatctgaggaacgtgaccccccccaaggtgaccctgttcgagcccagcaaggccgagatcgccaacaagcagaaagccaccctggtctgcctggccaggggcttcttccccgaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagctgaggtaccgagctcgaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcaga

SEQ ID NO: 206 - DNA, 3492 bp, Artificial Sequence
  synthetic
tgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgcatgcctgcaggtcgactctagaggatccaccggtcgccaccatgcgccaggtggcccgcgtgatcgtgttcctgaccctgagcaccctgagcctggccaagaccacccagcccatcagcatggacagctacgagggccaggaggtgaacatcacctgcagccacaacaacatcgccaccaacgactacatcacctggtaccagcagttccccagccagggcccccgcttcatcatccagggctacaagaccaaggtgaccaacgaggtggccagcctgttcatccccgccgaccgcaagagcagcaccctgagcctgccccgcgtgagcctgagcgacaccgccgtgtactactgcgcagccagcgacaactaccagctgatctggggcagcggcaccaagctgatcatcaagcccgacatccagaaccccgagcccgccgtgtaccagctgaaggaccccagaagccaggacagcaccctgtgcctgttcaccgacttcgacagccagatcaacgtgcccaagaccatggaaagcggcaccttcatcaccgacaagacagtgctggacatgaaggccatggacagcaagagcaacggcgccattgcctggtccaaccagaccagcttcacatgccaggacatcttcaaagagacaaacgccacctaccccagcagcgacgtgccctgcgacgccaccctgaccgagaagagcttcgagacagacatgaacctgaatttccagaacctgagcgtgatgggcctgcggatcctgctgctgaaggtggccggcttcaacctgctgatgaccctgcggctgtggagcagctgaggtaccgagctcgaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcg

SEQ ID NO: 207 - DNA, 3615 bp, Artificial Sequence
  synthetic
tgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggagacaagcttgcatgcctgcaggtcgactctagaggatccaccggtcgccaccatgggatgcagactgctgtgctgcgccgtgctgtgtctgctgggcgccgtgcccatcgacaccgaagtgacccagacccccaagcacctggtcatgggcatgaccaacaagaaaagcctgaagtgcgagcagcacatgggccaccgggccatgtactggtacaagcagaaggccaagaaaccccccgagctgatgttcgtgtacagctacgagaagctgagcatcaacgagagcgtgcccagccggttcagccccgagtgccccaatagcagcctgctgaacctgcatctgcacgccctgcagcccgaggacagcgccctgtatctgtgcgcaagcagccgcgccaactacgagcagtacttcggccccggcacccgcctgaccgtgctggaagatctgaggaacgtgaccccccccaaggtgaccctgttcgagcccagcaaggccgagatcgccaacaagcagaaagccaccctggtctgcctggccaggggcttcttccccgaccacgtggagctgtcttggtgggtgaacggcaaagaggtgcacagcggagtcagtaccgacccccaggcctacaaagagagcaactacagctactgcctgagcagcaggctgagagtgagcgccaccttctggcacaacccccggaaccacttccggtgccaggtgcagttccacggcctgagcgaagaggacaagtggcctgagggcagccccaagcccgtgacccagaacatcagcgccgaggcctggggcagagccgactgcggcatcaccagcgccagctaccaccagggcgtgctgtccgccaccatcctgtacgagatcctgctgggcaaggccaccctgtacgccgtgctggtgtccggcctggtgctgatggccatggtgaagaagaagaacagctgaggtaccgagctcgaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcg

SEQ ID NO: 208 - DNA, 6302 bp, Artificial Sequence
  synthetic
tcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagctcacttacaggcggccgcaccggtcgccaccatgagcctgtctagcctgctgaaggtcgtgaccgccagcctgtggctgggacctggaatcgcccagaagatcacccagacccagcccggcatgttcgtgcaggaaaaagaagccgtgaccctggactgcacctacgacaccagcgaccctagctacggcctgttctggtacaagcagcccagcagcggcgagatgatcttcctgatctaccagggcagctacgaccagcagaacgccaccgagggccggtacagcctgaacttccagaaggcccggaagtccgccaacctcgtgatcagcgctagccagctgggcgacagcgccatgtacttttgcgcaatcagcaacaccggcaaccagttctacttcggcaccggcaccagcctgaccgtgatccccaacatccagaatccggaccccgccgtgtaccagctgagagacagcaagagcagcgacaacactgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaatttccagaacctgagcgtgatcggcttcagaatcctgctgctgaaggtggccggcttcaacctgctgatgaccctgcggctgtggtccagctgaacgcgtatcgatgaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcaga

SEQ ID NO: 209 - DNA, 6413 bp, Artificial Sequence
  synthetic
cacttacaggcggccgcaccggtcgccaccatgggccctcagctgctgggatacgtggtgctgtgtctgctgggagccggacctctggaagcccaagtgacccagaaccccagatacctgatcaccgtgaccggcaagaaactgaccgtgacctgcagccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgcaagcaacaacctggccagctacaacgagcagttcttcggccctggcacccggctgaccgtgctggaagatctgaagaacgtgttccccccagaggtgaccgtgttcgagcctagcgaggccgagatcagccacacccagaaagccaccctcgtgtgcctggccaccggcttctatcccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccggggctgaacgcgtatcgatgaattcgagcatcttaccgccatttattcccatatttgttctgtttttcttgatttgggtatacatttaaatgttaataaaacaaaatggtggggcaatcatttacattttatgggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattccaccgtgaactttgtctcctggcatgcaaatcgtcaacttggcatgccaagaattaattcggatccaagcttaggcctgctcgctttcttgctgtcccatttctattaaaggttcctttgttccctaagtccaactactaaactgggggatattatgaagggccttgagcatctggattctgcctagcgctaagcttaacacgagccatagatagaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactgcccacctcgggggtctttcattctcgagcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcccaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaacattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgggcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattagtactctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttggctaactagatctgtatctggcggtcccgcggaagaactgacgagttcgtattcccggccgcagcccctgggagacgtcccagcggcctcgggggcccgttttgtggcccattctgtatcagttaacctacccgagtcggactttttggagctccgccactgtccgaggggtacgtggctttgttgggggacgagagacagagacacttcccgcccccgtctgaatttttgctttcggttttacgccgaaaccgcgccgcgcgtcttgtctgctgcagcatcgttctgtgttgtctctgtctgactgtgtttctgtatttgtctgaaaattagctcgacaaagttaagtaatagtccctctctccaagct

SEQ ID NO: 210 - DNA, 561 bp, Homo sapiens
atgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtacttctgtgcaatttcgaacaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatagtcagg

SEQ ID NO: 211 - DNA, 598 bp, Homo sapiens
atgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcaataacttagcctcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaagagcagcgctt

SEQ ID NO: 212 - protein, 11 aa, Rattus norvegicus
QSVSISRHNLI

SEQ ID NO: 213 - skipped

SEQ ID NO: 214 - protein, 7 aa, Rattus norvegicus
QQSGESP

SEQ ID NO: 215 - protein, 8 aa, Rattus norvegicus
GYTFTENY

SEQ ID NO: 216 - protein, 8 aa, Rattus norvegicus
IDPEDGTT

SEQ ID NO: 217 - protein, 8 aa, Rattus norvegicus
ARGVGSGD

SEQ ID NO: 218 - protein, 99 aa, Rattus norvegicus
LSLGQRATISCRASQSVSISRHNLIHWYQQKPGQQPKLLIYRASNLASGIPARFSGSGSGTDFTLTINPVQADDVATYYCQQSGESPRTFGGGTKLELK

SEQ ID NO: 219 - protein, 108 aa, Rattus norvegicus
GRGRAQSVVVQASGYTFTENYIYWVKQRPKQGLELIGRIDPEDGTTDYVEKFKNKATLTVDTSSKTAYMQLSSLTSEDTASYFCARGVGSGDYVMDAWGQGASVTVSS

SEQ ID NO: 220 - protein, 114 aa, Rattus norvegicus
LSLGQRATISCRASQSVSISRHNLIHWYQQKPGQQPKLLIYRASNLASGIPARFSGSGSGTDFTLTINPVQADDVATYYCQQSGESPRTFGGGTKLELKRADAAPTVPHLPQLC

SEQ ID NO: 221 - protein, 360 aa, Rattus norvegicus
GRGRAQSVVVQASGYTFTENYIYWVKQRPKQGLELIGRIDPEDGTTDYVEKFKNKATLTVDTSSKTAYMQLSSLTSEDTASYFCARGVGSGDYVMDAWGQGASVTVSSAETTAPSVYPLAPGTALKSNSMVTLGCLVKGYFPEPVTVTWNSGALSSGVHTFPAVLQSGLYTLTSSVTVPSSTWSSQAVTCNVTHPASSTKVDKKIVPRECNPCGCTGSEVSSVFIFPPKTKDVLTITLTPKVTCVVVDISQNDPEVRFSWFIDDVKVHTAQTHAPEKQSNSTLRSVSELPIVHRDWLNGKTFKCKVNSGAFPAPIEKASPNPSTPRGPQYTPCASQRDDPESVSITACKASIPRHLYGVR

SEQ ID NO: 222 - DNA, 423 bp, Rattus norvegicus
gagatgctgtctctagggcagagggccacaatctcctgtagggccagccaaagtgtcagtatatctagacataatcttatacactggtaccaacagaaaccaggacagcaacccaaactcctcatctaccgtgcatccaatctagcatctgggatccctgccaggttcagtggcagtgggtctgggacagacttcaccctcaccatcaatcctgtgcaggctgatgatgttgcaacctattactgtcagcagagtggggagtctcctcggacgttcggtggaggcaccaagctggaattgaaaagggctgatgctgcaccaactgtaccccatcttccccaactctgctgacagtaataggttgcaacatctcgcctgcacaggattgattgtgagggtgaagtctgttccatacctactgtg

SEQ ID NO: 223 - DNA, 1107 bp, Rattus norvegicus
gcggtcgaggtcgggctcagtcagttgtcgtgcaggcttctggctatacctttacagaaaattatatatactgggtgaagcagaggcctaaacagggcctggaattaataggaaggattgatcctgaagacggtactactgattatgttgagaagttcaaaaacaaggccacactgacagtagatacatcgtccaagacagcctacatgcaactcagcagcctgacatctgaggacacagcatcctatttttgtgccagaggtgtggggtccggggactatgttatggatgcctggggtcaaggagcttcagtcactgtctcctcagctgaaacaacagccccatctgtctatccactggctcctggaactgctctcaaaagtaactccatggtgaccctgggatgcctggtcaagggctatttccctgagccagtcaccgtgacctggaactctggagccctgtccagcggtgtgcacaccttcccagctgtcctgcagtctggactctacactctcaccagctcagtgactgtaccctccagcacctggtccagccaggccgtcacctgcaacgtaacccacccggccagcagcaccaaggtggacaagaaaattgtgccaagggaatgcaatccttgtggatgtacaggctcagaagtatcatctgtcttcatcttccccccaaagaccaaagatgtgctcaccatcactctgactccaaaggtcacgtgtgttgtggtagacattagccagaatgatcccgaggtccggttcagctggtttatagatgacgtgaaagtccacacagctcagactcatgccccggagaagcagtccaacagcactttacgctcagtcagtgaactccccatcgtgcaccgggactggctcaatggcaagacgttcaaatgcaaagtcaacagtggagcattccctgcccccatcgagaaagcatctccaaacccgagcacaccacgaggtccacagtatacaccatgcgcctcccagagagatgacccagagtcagtcagtatcactgcatgtaaagcttctatccccagacatttatacggagtgagatgacgggcagccacagaaactacag

SEQ ID NO: 224 - protein, 218 aa, Artificial Sequence
  humanized light chain
EIVLTQSPATLSLSPGQRATISCRASQSVSISRHNLIHWYQQKPGQAPRLLIYRASNRATGIPARFSGSGSGTDFTLTINPVQAEDFAVYYCQQSGESPRTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 444 aa, Artificial Sequence
  humanized heavy chain
EVQLVQSGAEVKKPGRTVKIVCQASGYTFTENYIYWVQQAPGKGLEWMGRIDPEDGTTDYAEKFQGRVTITADTSTDTAYMELSSLRSEDTAVYFCARGVGSGDYVMDAWGQGASVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 226 - protein, 10 aa, Rattus norvegicus
QSVSISGINL

SEQ ID NO: 227 - skipped

SEQ ID NO: 228 - protein, 10 aa, Rattus norvegicus
CQQSWESPRT

SEQ ID NO: 229 - protein, 8 aa, Rattus norvegicus
GYTFTAYY

SEQ ID NO: 230 - protein, 8 aa, Rattus norvegicus
IDPEDGST

SEQ ID NO: 231 - protein, 7 aa, Rattus norvegicus
CARGNSD

SEQ ID NO: 232 - protein, 100 aa, Rattus norvegicus
LCLRQRATISCRASQSVSISGINLMHWYQQRPGQQPKLLIYRASSLASGIPARFSGRGSGTDFTLTIDPVQADDIAAYFCQQSWESPRTFGGGTQLELKR

SEQ ID NO: 233 - protein, 109 aa, Rattus norvegicus
DWNSRTGLSQVSCKASGYTFTAYYISWVKQRPKQGLELIGRIDPEDGSTDYVEKFKIKATLTADTSSNTAYMQFSSLTFEDTATYFCARGNSDYVMDAWGQGASVTVSS

SEQ ID NO: 234 - protein, 107 aa, Rattus norvegicus
LCLRQRATISCRASQSVSISGINLMHWYQQRPGQQPKLLIYRASSLASGIPARFSGRGSGTDFTLTIDPVQADDIAAYFCQQSWESPRTFGGGTQLELKRADAAPTV

SEQ ID NO: 235 - protein, 407 aa, Rattus norvegicus
DWNSRTGLSQVSCKASGYTFTAYYISWVKQRPKQGLELIGRIDPEDGSTDYVEKFKIKATLTADTSSNTAYMQFSSLTFEDTATYFCARGNSDYVMDAWGQGASVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPRECNPCGCTGSEVSSVFIFPPKTKDVLTITLTPKVTCVVVDISQNDPEVRFSWFIDDVEVHTAQTHAPEKQSNSTLRSVSELPIVHRDWLNGKTFKCKVNSGAFPAPIEKSISKPEGTPRGPQVYTMAPPKEEMTQSQVSITCMVKGFYPPDIYTEWKMNGQPQENYKNTPPTMDTDGSYFLYSKLNVKKETCSRKHLR

SEQ ID NO: 236 - DNA, 746 bp, Rattus norvegicus
gcatgctgtgtctcaggcagagggccaccatctcctgtagggccagccagagtgtcagtatatctggcattaatttgatgcactggtaccaacagagaccaggacagcaacccaaactcctcatctatcgtgcatccagcctagcatctgggatccctgccaggttcagtggccgtgggtctgggacagacttcaccctcaccatcgatcctgtgcaggctgatgatattgcagcctatttctgtcagcagagttgggagtctcctcggacgttcggtggaggcacccagctggaattgaaacgggctgatgctgcaccaactgtacccatcttccacaattgtcggatctttggggacggggggattttgggatcctctagcccaccaagcgtcttccgtgcacgctcccgggggatatgacttatatctcccgcccctagacctggatgtgagatagctgattcgaatctgacaccaaggtgtatagcgggaattttgtgccgatggagatgctttacttttttggcgtcgtcgcgatggaaacggacagagctaacagcctctattttagttcaccacatagtagtgaaaccataccggcgcaacagtatcgcatgatactcagatgataacgctctggaccctctaattactctacgattactgctattctatcatcaggaggggtgaaggtccggtatatcgaagtcggttttccatctctcgcctctctgcgattgtcat

SEQ ID NO: 237 - DNA, 1221 bp, Rattus norvegicus
gactggaattcgaggacggggctcagtcaagtgtcgtgcaaggcttctggctatacctttacagcatactatatatcctgggtgaagcagaggcctaaacagggcctggaattaataggaaggattgatcctgaagacggtagtactgattatgttgagaagttcaaaatcaaggccacactgactgcagatacatcgtccaacacagcctacatgcaattcagcagcctgacatctgaggacacagcaacctatttctgtgctagagggaattcggactacgttatggatgcctggggtcaaggagcttcagtcactgtctcctcagctgaaacaacagccccatctgtctatccactggctcctggaactgctctcaaaagtaactccatggtgaccctgggatgcctggtcaagggctatttccctgagccagtcaccgtgacctggaactctggagccctgtccagcggtgtgcacaccttcccagctgtcctgcagtctggactctacactctcaccagctcagtgactgtaccctccagcacctggtccagccaggccgtcacctgcaacgtagcccacccggccagcagcaccaaggtggacaagaaaattgtgccaagggaatgcaatccttgtggatgtacaggctcagaagtatcatctgtcttcatcttccccccaaagaccaaagatgtgctcaccatcactctgactcctaaggtcacgtgtgttgtggtagacattagccagaatgatcccgaggtccggttcagctggtttatagatgacgtggaagtccacacagctcagactcatgccccggagaagcagtccaacagcactttacgctcagtcagtgaactccccatcgtgcaccgggactggctcaatggcaagacgttcaaatgcaaagtcaacagtggagcattccctgcccccatcgagaaaagcatctccaaacccgaaggcacaccacgaggtccacaggtatacaccatggcgcctcccaaggaagagatgacccagagtcaagtcagtatcacctgcatggtaaaaggcttctatcccccagacatttatacggagtggaagatgaacgggcagccacaggaaaactacaagaacactccacctacgatggacacagatgggagttacttcctctacagcaagctcaatgtaaagaaagaaacatgcagcaggaaacacttaagg

SEQ ID NO: 238 - protein, 5 aa, Artificial Sequence
  synthetic
GGGGS

SEQ ID NO: 239 - DNA, 75 bp, Artificial Sequence
  synthetic
gcaatcagcaacaccggcaaccagttctacttcggcaccggcaccagcctgaccgtgatccccaacatccagaat

SEQ ID NO: 240 - DNA, 79 bp, Artificial Sequence
  synthetic
ccggattctggatgttggggatcacggtcaggctggtgccggtgccgaagtagaactggttgccggtgttgctgattgc

SEQ ID NO: 241 - DNA, 96 bp, Artificial Sequence
  synthetic
gcaagcaacaacctggccagctacaacgagcagttcttcggccctggcacccggctgaccgtgctggaagatctgaagaacgtgttccccccagag

SEQ ID NO: 242 - DNA, 101 bp, Artificial Sequence
  synthetic
gtcacctctggggggaacacgttcttcagatcttccagcacggtcagccgggtgccagggccgaagaactgctcgttgtagctggccaggttgttgcttgc

SEQ ID NO: 243 - DNA, 30 bp, Homo sapiens
gcaatttcgaacaccggtaaccagttctat

SEQ ID NO: 244 - DNA, 36 bp, Homo sapiens
gccagcaataacttagcctcctacaatgagcagttc

SEQ ID NO: 245 - protein, 10 aa, Homo sapiens
AISNTGNQFY

SEQ ID NO: 246 - protein, 12 aa, Homo sapiens
ASNNLASYNEQF

SEQ ID NO: 247 - DNA, 825 bp, Artificial Sequence
  synthetic
atgagcctgtctagcctgctgaaggtcgtgaccgccagcctgtggctgggacctggaatcgcccagaagatcacccagacccagcccggcatgttcgtgcaggaaaaagaagccgtgaccctggactgcacctacgacaccagcgaccctagctacggcctgttctggtacaagcagcccagcagcggcgagatgatcttcctgatctaccagggcagctacgaccagcagaacgccaccgagggccggtacagcctgaacttccagaaggcccggaagtccgccaacctcgtgatcagcgctagccagctgggcgacagcgccatgtacttttgcgcaatcagcaacaccggcaaccagttctacttcggcaccggcaccagcctgaccgtgatccccaacatccagaatccggaccccgccgtgtaccagctgagagacagcaagagcagcgacaacactgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaatttccagaacctgagcgtgatcggcttcagaatcctgctgctgaaggtggccggcttcaacctgctgatgaccctgcggctgtggtccagctga

SEQ ID NO: 248 - DNA, 936 bp, Artificial Sequence
  synthetic
atgggccctcagctgctgggatacgtggtgctgtgtctgctgggagccggacctctggaagcccaagtgacccagaaccccagatacctgatcaccgtgaccggcaagaaactgaccgtgacctgcagccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgcaagcaacaacctggccagctacaacgagcagttcttcggccctggcacccggctgaccgtgctggaagatctgaagaacgtgttccccccagaggtgaccgtgttcgagcctagcgaggccgagatcagccacacccagaaagccaccctcgtgtgcctggccaccggcttctatcccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccggggctga

SEQ ID NO: 249 - protein, 274 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAISNTGNQFYFGTGTSLTVIPNIQNPDPAVYQLRDSKSSDNTVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 250 - protein, 311 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASNNLASYNEQFFGPGTRLTVLEDLKNVFPPEVTVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 251 - DNA, 3551 bp, Artificial Sequence
  synthetic
acaagcttgcatgcctgcaggtcgactctagaggatccaccggtcgccaccatgagcctgtctagcctgctgaaggtcgtgaccgccagcctgtggctgggacctggaatcgcccagaagatcacccagacccagcccggcatgttcgtgcaggaaaaagaagccgtgaccctggactgcacctacgacaccagcgaccctagctacggcctgttctggtacaagcagcccagcagcggcgagatgatcttcctgatctaccagggcagctacgaccagcagaacgccaccgagggccggtacagcctgaacttccagaaggcccggaagtccgccaacctcgtgatcagcgctagccagctgggcgacagcgccatgtacttttgcgcaatcagcaacaccggcaaccagttctacttcggcaccggcaccagcctgaccgtgatccccaacatccagaatccggaccccgccgtgtaccagctgagagacagcaagagcagcgacaacactgtgtgcctgttcaccgacttcgactcccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttcccaagccccgagagcagctgcgacgtgaagctggtggaaaagagcttcgagacagacaccaacctgaatttccagaacctgagcgtgatcggcttcagaatcctgctgctgaaggtggccggcttcaacctgctgatgaccctgcggctgtggtccagctgaacgcgtatcgatgaattccacgctagccacgatatcaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcgtgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggag

SEQ ID NO: 252 - DNA, 3662 bp, Artificial Sequence
  synthetic
acaagcttgcatgcctgcaggtcgactctagaggatccaccggtcgccaccatgggccctcagctgctgggatacgtggtgctgtgtctgctgggagccggacctctggaagcccaagtgacccagaaccccagatacctgatcaccgtgaccggcaagaaactgaccgtgacctgcagccagaacatgaaccacgagtacatgagctggtacagacaggaccccggcctgggcctgcggcagatctactacagcatgaacgtggaagtgaccgacaagggcgacgtgcccgagggctacaaggtgtcccggaaagagaagcggaacttcccactgatcctggaaagccccagccccaaccagaccagcctgtacttctgcgcaagcaacaacctggccagctacaacgagcagttcttcggccctggcacccggctgaccgtgctggaagatctgaagaacgtgttccccccagaggtgaccgtgttcgagcctagcgaggccgagatcagccacacccagaaagccaccctcgtgtgcctggccaccggcttctatcccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacccagatcgtgtctgccgaagcctggggcagagccgattgcggctttaccagcgagagctaccagcagggcgtgctgagcgccaccatcctgtacgagatcctgctgggcaaggccaccctgtacgccgtgctggtgtctgccctggtgctgatggctatggtcaagcggaaggacagccggggctgaacgcgtatcgatgaattccacgctagccacgatatcaattcaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaaaaaaaaaaaaaaaaaaaaaaaaaaaaaaaaaaactagtggcgtgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatgccaacaacgttgcacaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatcgaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactcgaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctaatacgactcactatagggag

SEQ ID NO: 253 - protein, 10 aa, Artificial Sequence
  humanized version
QSVSISGINL

SEQ ID NO: 254 - protein, 10 aa, Artificial Sequence
  humanized version
CQQSWESPRT

SEQ ID NO: 255 - protein, 8 aa, Artificial Sequence
  humanized version
GYTFTAYY

SEQ ID NO: 256 - protein, 7 aa, Artificial Sequence
  humanized version
DPEDGST

SEQ ID NO: 257 - protein, 12 aa, Artificial Sequence
  humanized version
CARGNSDYGMDV

SEQ ID NO: 258 - protein, 111 aa, Artificial Sequence
  humanized version
EIVMTQSPATLSLSPGERATLSCRASQSVSISGINLLSWYQQKPGQAPRLLIYRASTRATGIPARFSGSGSGTDFTLTISSVQAEDIAVYYCQQSWESPRTFGGGTKLEIK

SEQ ID NO: 259 - protein, 118 aa, Artificial Sequence
  humanized version
EVQLVQSGAEVKKPGATVKISCKASGYTFTAYYMHWVQQAPGKGLEWMGRIDPEDGSTIYAEKFQGRVTLTADTSTDTAYMELSSLRSEDTAVYFCARGNSDYGMDVWGQGTTVTVSS

SEQ ID NO: 260 - protein, 218 aa, Artificial Sequence
  humanized version
EIVMTQSPATLSLSPGERATLSCRASQSVSISGINLLSWYQQKPGQAPRLLIYRASTRATGIPARFSGSGSGTDFTLTISSVQAEDIAVYYCQQSWESPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 448 aa, Artificial Sequence
  humanized version
EVQLVQSGAEVKKPGATVKISCKASGYTFTAYYMHWVQQAPGKGLEWMGRIDPEDGSTIYAEKFQGRVTLTADTSTDTAYMELSSLRSEDTAVYFCARGNSDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 238 aa, Artificial Sequence
  humanized version
MYRMQLLSCIALSLALVTNSEIVMTQSPATLSLSPGERATLSCRASQSVSISGINLLSWYQQKPGQAPRLLIYRASTRATGIPARFSGSGSGTDFTLTISSVQAEDIAVYYCQQSWESPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 468 aa, Artificial Sequence
  humanized version
MYRMQLLSCIALSLALVTNSEVQLVQSGAEVKKPGATVKISCKASGYTFTAYYMHWVQQAPGKGLEWMGRIDPEDGSTIYAEKFQGRVTLTADTSTDTAYMELSSLRSEDTAVYFCARGNSDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - DNA, 657 bp, Artificial Sequence
  humanized version
gaaattgtgatgacccagagcccggcgaccctgagcctgagcccgggcgaacgcgcgaccctgagctgccgcgcgagccagagcgtgagcattagcggcattaacctgctgagctggtatcagcagaaaccgggccaggcgccgcgcctgctgatttatcgcgcgagcacccgcgcgaccggcattccggcgcgctttagcggcagcggcagcggcaccgattttaccctgaccattagcagcgtgcaggcggaagatattgcggtgtattattgccagcagagctgggaaagcccgcgcacctttggcggcggcaccaaactggaaattaaaaggacagtggccgctcccagcgtgttcatcttcccacctagcgacgaacagctgaagtctggcaccgcctccgtcgtgtgcctgctgaacaacttctacccccgcgaggccaaggtgcagtggaaagtggacaatgccctgcagtccggcaacagccaggaaagcgtgaccgagcaggactccaaggatagcacctacagcctgagcagcaccctgacactgagcaaggccgactacgagaagcacaaggtgtacgcctgtgaagtgacccaccagggcctgtccagcccagtgaccaagagcttcaaccggggcgagtgctga

SEQ ID NO: 265 - DNA, 1347 bp, Artificial Sequence
  humanized version
gaagtgcagctggtgcagagcggcgcggaagtgaaaaaaccgggcgcgaccgtgaaaattagctgcaaagcgagcggctatacctttaccgcgtattatatgcattgggtgcagcaggcgccgggcaaaggcctggaatggatgggccgcattgatccggaagatggcagcaccatttatgcggaaaaatttcagggccgcgtgaccctgaccgcggataccagcaccgataccgcgtatatggaactgagcagcctgcgcagcgaagataccgcggtgtatttttgcgcgcgcggcaacagcgattatggcatggatgtgtggggccagggcaccaccgtgaccgtgagcagcgcctctacaaagggcccctccgtgttccctctggcccctagcagcaagagcacatctggcggaacagccgccctgggctgcctcgtgaaggactactttcccgagcccgtgaccgtgtcctggaacagcggagcactgacaagcggcgtgcacacctttccagccgtgctgcagtctagcggcctgtacagcctgtcctccgtcgtgactgtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctggctggccctgacgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccgagtctaaggccaagggacagccccgcgagccccaggtgtacacactgcctccaagccgggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtactccaagctgaccgtggacaagtcccggtggcagcagggcaacgtgttcagctgctccgtgatgcacgaggccctgcacaaccactacacccagaagtccctgtccctgagccccggcaaatac

SEQ ID NO: 266 - protein, 10 aa, Artificial Sequence
  chimeric version
QSVSISGINL

SEQ ID NO: 267 - protein, 10 aa, Artificial Sequence
  chimeric version
CQQSWESPRT

SEQ ID NO: 268 - protein, 8 aa, Artificial Sequence
  chimeric version
GYTFTAYY

SEQ ID NO: 269 - protein, 8 aa, Artificial Sequence
  chimeric version
IDPEDGST

SEQ ID NO: 270 - protein, 12 aa, Artificial Sequence
  chimeric version
CARGNSDYVMDA

SEQ ID NO: 271 - protein, 110 aa, Artificial Sequence
  chimeric version
DIVLTQSPVLAVSLGQRATISCRASQSVSISGINLMHWYQQRPGQQPKLLIYRASSLASGIPARFSGRGSGTDFTLTIDPVQADDIAAYFCQQSWESPRTFGGGTQLELK

SEQ ID NO: 272 - protein, 118 aa, Artificial Sequence
  chimeric version
EVQLQQSGPELQRPGASVKLSCKASGYTFTAYYISWVKQRPKQGLELIGRIDPEDGSTDYVEKFKIKATLTADTSSNTAYMQFSSLTSEDTATYFCARGNSDYVMDAWGQGASVTVSS

SEQ ID NO: 273 - protein, 217 aa, Artificial Sequence
  chimeric version
DIVLTQSPVLAVSLGQRATISCRASQSVSISGINLMHWYQQRPGQQPKLLIYRASSLASGIPARFSGRGSGTDFTLTIDPVQADDIAAYFCQQSWESPRTFGGGTQLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 448 aa, Artificial Sequence
  chimeric version
EVQLQQSGPELQRPGASVKLSCKASGYTFTAYYISWVKQRPKQGLELIGRIDPEDGSTDYVEKFKIKATLTADTSSNTAYMQFSSLTSEDTATYFCARGNSDYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 237 aa, Artificial Sequence
  chimeric version
MYRMQLLSCIALSLALVTNSDIVLTQSPVLAVSLGQRATISCRASQSVSISGINLMHWYQQRPGQQPKLLIYRASSLASGIPARFSGRGSGTDFTLTIDPVQADDIAAYFCQQSWESPRTFGGGTQLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 468 aa, Artificial Sequence
  chimeric version
MYRMQLLSCIALSLALVTNSEVQLQQSGPELQRPGASVKLSCKASGYTFTAYYISWVKQRPKQGLELIGRIDPEDGSTDYVEKFKIKATLTADTSSNTAYMQFSSLTSEDTATYFCARGNSDYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - DNA, 654 bp, Artificial Sequence
  chimeric version
gacatcgtgctgacccagagccccgtgctggctgtgtctctgggacagagagccaccatcagctgtcgggcctctcagagcgtgtccatcagcggcatcaacctgatgcactggtatcagcagcgccctggccagcagcccaagctgctgatctacagagccagctccctggcctctggcatccccgccagattttctggcagaggctccggcaccgactttaccctgaccatcgaccctgtgcaggccgacgatattgccgcctacttctgccagcagagctgggagagccccagaacctttggcggcggaacccagctggaactgaagaggacagtggccgctcccagcgtgttcatcttcccacctagcgacgaacagctgaagtctggcaccgcctccgtcgtgtgcctgctgaacaacttctacccccgcgaggccaaggtgcagtggaaagtggacaatgccctgcagtccggcaacagccaggaaagcgtgaccgagcaggactccaaggatagcacctacagcctgagcagcaccctgacactgagcaaggccgactacgagaagcacaaggtgtacgcctgtgaagtgacccaccagggcctgtccagcccagtgaccaagagcttcaaccggggcgagtgctga

SEQ ID NO: 278 - DNA, 1347 bp, Artificial Sequence
  chimeric version
gaagtgcagctgcagcagagcggccctgagctgcagaggcctggcgcttctgtgaagctgtcctgcaaggccagcggctacaccttcaccgcctactacatcagctgggtcaagcagcggcccaagcagggcctggaactgatcggcagaatcgaccccgaggacggcagcaccgactacgtggaaaagttcaagatcaaggccaccctgaccgccgacaccagcagcaataccgcctacatgcagttcagcagcctgaccagcgaggacaccgccacctacttttgcgccagaggcaacagcgattacgtgatggacgcctggggacagggcgccagcgtgacagtgtctagcgcctctacaaagggcccctccgtgttccctctggcccctagcagcaagagcacatctggcggaacagccgccctgggctgcctcgtgaaggactactttcccgagcccgtgaccgtgtcctggaacagcggagcactgacaagcggcgtgcacacctttccagccgtgctgcagtctagcggcctgtacagcctgtcctccgtcgtgactgtgcccagcagctctctgggcacccagacctacatctgcaacgtgaaccacaagcccagcaacaccaaggtggacaagaaggtggaacccaagagctgcgacaagacccacacctgtcccccttgtcctgcccccgaactgctggctggccctgacgtgttcctgttccccccaaagcccaaggataccctgatgatcagccggacccccgaagtgacctgcgtggtggtggatgtgtcccacgaggaccctgaagtgaagttcaattggtacgtggacggcgtggaagtgcacaacgccaagaccaagcctagagaggaacagtacaacagcacctaccgggtggtgtccgtgctgacagtgctgcaccaggactggctgaacggcaaagagtacaagtgcaaggtgtccaacaaggccctgcctgcccccatcgagaaaaccgagtctaaggccaagggacagccccgcgagccccaggtgtacacactgcctccaagccgggaagagatgaccaagaaccaggtgtccctgacctgtctcgtgaaaggcttctacccctccgatatcgccgtggaatgggagagcaacggccagcccgagaacaactacaagaccaccccccctgtgctggacagcgacggctcattcttcctgtactccaagctgaccgtggacaagtcccggtggcagcagggcaacgtgttcagctgctccgtgatgcacgaggccctgcacaaccactacacccagaagtccctgtccctgagccccggcaaatac

SEQ ID NO: 279 - protein, 11 aa, Artificial Sequence
  chimeric version
QSVSISRHNLI

SEQ ID NO: 280 - protein, 10 aa, Artificial Sequence
  chimeric version
CQQSGESPRT

SEQ ID NO: 281 - protein, 8 aa, Artificial Sequence
  chimeric version
GYTFTENY

SEQ ID NO: 282 - protein, 8 aa, Artificial Sequence
  chimeric version
IDPEDGTT

SEQ ID NO: 283 - protein, 14 aa, Artificial Sequence
  chimeric version
CARGVGSGDYVMDA

SEQ ID NO: 284 - protein, 110 aa, Artificial Sequence
  chimeric version
DIVLTQSPALAVSLGQRATISCRASQSVSISRHNLIHWYQQKPGQQPKLLIYRASNLASGIPARFSGSGSGTDFTLTINPVQADDVATYYCQQSGESPRTFGGGTKLELK

SEQ ID NO: 285 - protein, 120 aa, Artificial Sequence
  chimeric version
EVQLQQSGPELQRPGRAQSVVCQASGYTFTENYIYWVKQRPKQGLELIGRIDPEDGTTDYVEKFKNKATLTVDTSSKTAYMQLSSLTSEDTASYFCARGVGSGDYVMDAWGQGASVTVSS

SEQ ID NO: 286 - protein, 217 aa, Artificial Sequence
  chimeric version
DIVLTQSPALAVSLGQRATISCRASQSVSISRHNLIHWYQQKPGQQPKLLIYRASNLASGIPARFSGSGSGTDFTLTINPVQADDVATYYCQQSGESPRTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 450 aa, Artificial Sequence
  chimeric version
EVQLQQSGPELQRPGRAQSVVCQASGYTFTENYIYWVKQRPKQGLELIGRIDPEDGTTDYVEKFKNKATLTVDTSSKTAYMQLSSLTSEDTASYFCARGVGSGDYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 288 - protein, 237 aa, Artificial Sequence
  chimeric version
MYRMQLLSCIALSLALVTNSDIVLTQSPALAVSLGQRATISCRASQSVSISRHNLIHWYQQKPGQQPKLLIYRASNLASGIPARFSGSGSGTDFTLTINPVQADDVATYYCQQSGESPRTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 470 aa, Artificial Sequence
  chimeric version
MYRMQLLSCIALSLALVTNSEVQLQQSGPELQRPGRAQSVVCQASGYTFTENYIYWVKQRPKQGLELIGRIDPEDGTTDYVEKFKNKATLTVDTSSKTAYMQLSSLTSEDTASYFCARGVGSGDYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - DNA, 654 bp, Artificial Sequence
  chimeric version
gatattgtgctgacccagagcccggcgctggcggtgagcctgggccagcgcgcgaccattagctgccgcgcgagccagagcgtgagcattagccgccataacctgattcattggtatcagcagaaaccgggccagcagccgaaactgctgatttatcgcgcgagcaacctggcgagcggcattccggcgcgctttagcggcagcggcagcggcaccgattttaccctgaccattaacccggtgcaggcggatgatgtggcgacctattattgccagcagagcggcgaaagcccgcgcacctttggcggcggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 291 - DNA, 1353 bp, Artificial Sequence
  chimeric version
gaagtgcagctgcagcagagcggcccggaactgcagcgcccgggccgcgcgcagagcgtggtgtgccaggcgagcggctatacctttaccgaaaactatatttattgggtgaaacagcgcccgaaacagggcctggaactgattggccgcattgatccggaagatggcaccaccgattatgtggaaaaatttaaaaacaaagcgaccctgaccgtggataccagcagcaaaaccgcgtatatgcagctgagcagcctgaccagcgaagataccgcgagctatttttgcgcgcgcggcgtgggcagcggcgattatgtgatggatgcgtggggccagggcgcgagcgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggcgggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccgaatccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 292 - protein, 11 aa, Artificial Sequence
  humanized version
QSVSISRHNLI

SEQ ID NO: 293 - protein, 10 aa, Artificial Sequence
  humanized version
CQQSGESPRT

SEQ ID NO: 294 - protein, 8 aa, Artificial Sequence
  humanized version
GYTFTENY

SEQ ID NO: 295 - protein, 8 aa, Artificial Sequence
  humanized version
IDPEDGTT

SEQ ID NO: 296 - protein, 14 aa, Artificial Sequence
  humanized version
CARGVGSGDYVMDA

SEQ ID NO: 297 - protein, 111 aa, Artificial Sequence
  humanized version
EIVLTQSPATLSLSPGQRATISCRASQSVSISRHNLIHWYQQKPGQAPRLLIYRASNRATGIPARFSGSGSGTDFTLTINPVQAEDFAVYYCQQSGESPRTFGGGTKLELK

SEQ ID NO: 298 - protein, 120 aa, Artificial Sequence
  humanized version
EVQLVQSGAEVKKPGRTVKIVCQASGYTFTENYIYWVQQAPGKGLEWMGRIDPEDGTTDYAEKFQGRVTITADTSTDTAYMELSSLRSEDTAVYFCARGVGSGDYVMDAWGQGASVTVSS

SEQ ID NO: 299 - protein, 218 aa, Artificial Sequence; identical to SEQ ID NO: 224
  humanized version

SEQ ID NO: 300 - protein, 450 aa, Artificial Sequence
  humanized version
EVQLVQSGAEVKKPGRTVKIVCQASGYTFTENYIYWVQQAPGKGLEWMGRIDPEDGTTDYAEKFQGRVTITADTSTDTAYMELSSLRSEDTAVYFCARGVGSGDYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 238 aa, Artificial Sequence
  humanized version
MYRMQLLSCIALSLALVTNSEIVLTQSPATLSLSPGQRATISCRASQSVSISRHNLIHWYQQKPGQAPRLLIYRASNRATGIPARFSGSGSGTDFTLTINPVQAEDFAVYYCQQSGESPRTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 302 - protein, 470 aa, Artificial Sequence
  humanized version
MYRMQLLSCIALSLALVTNSEVQLVQSGAEVKKPGRTVKIVCQASGYTFTENYIYWVQQAPGKGLEWMGRIDPEDGTTDYAEKFQGRVTITADTSTDTAYMELSSLRSEDTAVYFCARGVGSGDYVMDAWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTESKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - DNA, 657 bp, Artificial Sequence
  humanized version
gaaattgtgctgacccagagcccggcgaccctgagcctgagcccgggccagcgcgcgaccattagctgccgcgcgagccagagcgtgagcattagccgccataacctgattcattggtatcagcagaaaccgggccaggcgccgcgcctgctgatttatcgcgcgagcaaccgcgcgaccggcattccggcgcgctttagcggcagcggcagcggcaccgattttaccctgaccattaacccggtgcaggcggaagattttgcggtgtattattgccagcagagcggcgaaagcccgcgcacctttggcggcggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 304 - DNA, 1353 bp, Artificial Sequence
  humanized version
gaagtgcagctggtgcagagcggcgcggaagtgaaaaaaccgggccgcaccgtgaaaattgtgtgccaggcgagcggctatacctttaccgaaaactatatttattgggtgcagcaggcgccgggcaaaggcctggaatggatgggccgcattgatccggaagatggcaccaccgattatgcggaaaaatttcagggccgcgtgaccattaccgcggataccagcaccgataccgcgtatatggaactgagcagcctgcgcagcgaagataccgcggtgtatttttgcgcgcgcggcgtgggcagcggcgattatgtgatggatgcgtggggccagggcgcgagcgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggcgggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccgaatccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2018/0256716 A1
(43) Pub. Date: Sep. 13, 2018

(54) T-CELL RECEPTOR SPECIFIC ANTIBODIES

(71) Applicant: MEDIGENE IMMUNOTHERAPIES GMBH, Planegg-Martinsried (DE)

(72) Inventors: Dolores SCHENDEL, München (DE); Slavoljub MILOSEVIC, München (DE); Tanja HERRMANN, München (DE); Michaela KÜGLER, Warthausen (DE)

(21) Appl. No.: 15/579,117

(22) PCT Filed: Jun. 1, 2016
(86) PCT No.: PCT/EP2016/062370

§ 371 Date: Dec. 1, 2017

(30) Foreign Application Priority Data
Jun. 1, 2015 (EP) 15170154.7

(51) Int. Cl. A61K 39/395; A61P 35/02; A61K 47/68

(52) U.S. Cl. CPC A61K 39/39558; A61P 35/02; A61K 2039/507; A61K 47/6851; A61K 47/6809

(57) ABSTRACT

The present invention relates to an antibody or binding fragment thereof that binds to a fraction of T cell receptor variable alpha (TCR Vα) chains comprising at least two different TCR Vα chains but less than all TCR Vα chains or that binds to a fraction of T cell receptor variable beta (TCR Vβ) chains comprising at least two different TCR Vβ chains but less than all TCR Vβ chains.

### FIELD OF THE INVENTION

The present invention relates to an antibody or a binding fragment thereof that binds to a fraction of T cell receptor variable alpha (TCR Vα) chains comprising at least two different TCR Vα chains but less than all TCR Vα chains or that binds to a fraction of T cell receptor variable beta (TCR Vβ) chains comprising at least two different TCR Vβ chains but less than all TCR Vβ chains. Further, the invention relates to use of an antibody or binding fragment thereof for depleting a subpopulation of T cells.

### BACKGROUND OF THE INVENTION

With their central role in the immune system, T cells normally provide protection from pathogens or malignant cells. Each T cell expresses a single form of a T cell receptor (TCR)—the structure which a T cell uses to recognize infected or modified cells. In some cases, however, T cells can be pathogenic and attack and destroy healthy tissues, as occurs in autoimmune diseases such as multiple sclerosis (MS). Furthermore, as rapidly dividing cells, T cells are susceptible to genetic mutations that lead to highly aggressive T cell leukemias (TCL).

Each TCR is a heterodimer composed of one variable alpha (Vα) chain and one variable beta (Vβ) chain. In turn, each chain is comprised of one constant (C) region and one variable (V) region. There are only one or two gene segments in the human genome encoding the constant regions of human α and β chains, respectively. In contrast, there are 45 different gene segments encoding the alpha variable regions (AV) that are used to generate the full repertoire of human Vα chains and 47 variable gene segments (BV) that encode the full repertoire of human Vβ chains (BV).

Different diseases are caused by unwanted T cells. For example T cells that can undergo malignant transformation leading to T cell leukemia. T cells with particular TCRs can become activated and attack normal body structures in an aberrant fashion as occurs in various forms of T cell mediated autoimmunity. Examples here are multiple sclerosis, insulin-dependent type 1 diabetes mellitus and psoriasis among others. Normal T cells can also mediate unwanted responses directed against normal allogeneic cells, as occurs in host-versus-graft responses where T cells present in a bone marrow or stem cell recipient attack donor cells that are transplanted to reconstitute a new hematopoietic system. Reciprocally, graft-versus-host disease can occur when transplanted donor T cells attack recipient tissues based on recognition of allogeneic differences. More recently, engineered T cells expressing transgenic TCRs have become agents for treatment of cancer or viral diseases. In some cases these T cells can lead to unwanted reactions in recipient patients, thereby becoming unwanted T cells because of their undesired toxicities. Antibodies binding to TCRs can be powerful tools to eliminate unwanted T cells from a patient.

Pan-specific TCR antibodies recognize all Vα or Vβ chains. Monoclonal antibodies (mabs) which are pan-specific may be used to deplete T cells that cause stem cell rejection in patients undergoing bone marrow/stem cell transplantation. A pan-specific mab could be used to deplete all residual patient T cells before transplantation.

Because these pan-specific mabs would lead to the deletion of all TCRs, they can only be used in limited situations because the immune system of the patient no longer can act via its TCR arm.

Mono-specific mabs recognizing individual TCR Vα or Vβ chains primarily have value as research tools. Their therapeutic usefulness is however limited today as, in order to target all possible unwanted T cells expressing specific TCRs it would be necessary to produce a large repertoire of different antibodies, i.e. 45 antibodies binding specifically to the 45 different Vα chains and 47 antibodies binding specifically to the 47 different Vβ chains. Thus a large repertoire of antibodies would have to be generated in order to allow the depletion of all possible unwanted T cells, based on their expression of specific TCRs.

There is thus a need for antibodies which allow for depletion of specific populations of TCRs and thus specific populations of T cells without depletion of the complete population of TCRs and not being subject to some of the aforementioned limitations.

### Objectives and Summary of the Invention

Against this background, it is one objective of the present invention to provide antibodies which allow for depletion of specific populations of TCRs and thus specific populations of T cells without depletion of the complete population of TCRs.

It is another objective of the present invention to provide antibodies or fragments thereof and their use in therapy.

In the context of the invention, an antibody is described that binds different TCR variable chains. In certain embodiments, the antibody may bind different TCR variable chains that belong to different subfamilies.

This antibody therefore seems capable of specifically depleting a subpopulation of T cells without depleting the complete repertoire of T cells. Thereby, a patient treated with such an antibody would remain immunocompetent to respond to pathogen challenges through the presence of remaining T cells untouched by the antibody. Such an antibody can be considered to be a cluster-specific TCR mab. It seems fair to conclude that other cluster-specific TCR mabs can be obtained using inter alia the approaches described hereinafter.

Against this background, the present application in a first aspect is concerned with an antibody or a binding fragment thereof that binds to a fraction of T cell receptor variable alpha (TCR Vα) chains comprising at least two different TCR Vα chains but less than all TCR Vα chains or that binds to a fraction of T cell receptor variable beta (TCR Vβ) chains comprising at least two different TCR Vβ chains but less than all TCR Vβ chains.

In preferred embodiments, the fraction of TCR Vα chains comprises at least two different TCR Vα chains that belong to at least two different TCR Vα chain subfamilies or wherein the fraction TCR Vβ chains comprises least to two different TCR Vβ chains that belong to at least two different TCR Vβ chain subfamilies.

In other preferred embodiments, the fraction of TCR Vα chains comprises at least 3, or at least 4, or at least 5, or at least 6 different TCR Vα chains or the fraction of TCR Vβ chains comprises at least 3, or at least 4, or at least 5, or at least 6 different TCR Vβ chains.

In another preferred embodiment, the fraction of TCR Vα chains comprises at least 5%, or at least 10%, or at least 25%, or at least 30%, or at least 40%, or at least 50% of the total amount TCR Vα chains expressed on T cells or wherein the fraction of TCR Vβ chains comprises at least 5%, or at least 10%, or at least 25%, or at least 30%, or at least 40%, or at least 50% of the total amount TCR Vβ chains expressed on T cells.

In a specific embodiment, the antibody or binding fragment thereof binds to a fraction of TCR Vβ chains.

In a further embodiment, the fraction comprises at least two different TCR Vβ chains that belong to at least two different subfamilies selected from the group comprising BV4, BV6, BV7, BV12, BV14, BV18, BV20, BV21, BV23, BV24 and BV29, in particular the group comprising BV12, BV14, BV18, BV24 and BV29.

In another embodiment, the fraction comprises at least two different TCR Vβ chains that belong to at least two different subfamilies selected from the group comprising BV7, BV12, BV 25 and BV 28.

In still another embodiment, the fraction comprises at least 2, preferably at least 3, more preferably at least 4 different TCR Vβ chains selected from the group comprising TRBV7-3, TRBV12-3, TRBV 12-4, TRBV 25-1, TRBV 28. In a specific embodiment the fraction comprises at least 2, preferably at least 3, more preferably at least 4 different TCR Vβ chains selected from the group comprising TRBV7-2, TRBV7-3, TRBV12-3, TRBV 12-4, TRBV 25-1, TRBV28. In another specific embodiment, the fraction comprises at least 2, preferably at least 3, more preferably at least 4 different TCR Vβ chains selected from the group comprising TRBV2, TRBV5-6, TRBV6-5, TRBV7-3, TRBV7-9, TRBV10-3, TRBV12-3, TRBV 12-4, TRBC 12-5, TRBV21-1, TRBV6-7, TRBV24-1, TRBV 25-1, TRBV28.

As will become apparent from the ensuing description the antibody or binding fragment according to the invention is capable of depleting a subpopulation of T cells expressing a fraction of TCR Vα chains comprising at least two different TCR Vα chains or is capable of depleting a subpopulation of T cells expressing a fraction of TCR Vβ chains comprising at least two different TCR Vβ chains.

An exemplary antibody of the invention is clone 15B4 comprising a light chain variable region and/or a heavy chain variable region, wherein the light chain variable region comprises at least a CDR1 set forth in SEQ ID No.: 212 or sequences at least 80% identical thereto, a CDR2 set forth in SEQ ID No.: 213 or sequences at least 80% identical thereto, and/or a CDR3 set forth in SEQ ID No.: 214 or sequences at least 80% identical thereto; and/or wherein the heavy chain variable region comprises at least a CDR1 set forth in SEQ ID No.: 215 or sequences at least 80% identical thereto, a CDR2 set forth in SEQ ID No.: 216 or sequences at least 80% identical thereto, and/or a CDR3 set forth in SEQ ID No.: 217 or sequences at least 80% identical thereto.

More specifically clone 15B4 comprises a light chain variable region comprising SEQ ID No.: 218 or sequences at least 80% identical thereto and/or a heavy chain variable region comprising SEQ ID No.: 219 or sequences at least 80% identical thereto.

Another exemplary antibody of the invention is clone 5H4 comprising a light chain variable region and/or a heavy chain variable region, wherein the light chain variable region comprises at least a CDR1 set forth in SEQ ID No.: 226 or sequences at least 80% identical thereto, a CDR2 set forth in SEQ ID No.: 227 or sequences at least 80% identical thereto, and/or a CDR3 set forth in SEQ ID No.: 228 or sequences at least 80% identical thereto; and/or wherein the heavy chain variable region comprises at least a CDR1 set forth in SEQ ID No.: 229 or sequences at least 80% identical thereto, a CDR2 set forth in SEQ ID No.: 230 or sequences at least 80% identical thereto, and/or a CDR3 set forth in SEQ ID No.: 231 or sequences at least 80% identical thereto.

More specifically clone 5H4 comprises a light chain variable region comprising SEQ ID No.: 232 or sequences at least 80% identical thereto and/or a heavy chain variable region comprising SEQ ID No.: 233 or sequences at least 80% identical thereto.

In other specific embodiments, the antibody or binding fragment thereof according to the invention is a monoclonal antibody or binding fragment thereof. The antibody may be a chimeric, humanized or human antibody or binding fragment thereof.

A further aspect of the invention relates to the use of an antibody or binding fragment thereof for depleting a subpopulation of T cells expressing a fraction of TCR Vα chains comprising at least two different TCR Vα chains or for depleting a subpopulation of T cells expressing a fraction of TCR Vβ chains comprising at least two different TCR Vβ chains.

Such cluster-specific TCR mabs may also be used for the depletion of unwanted or pathogenic T cells, for example mediating unwanted immune responses or T cells proliferating in an uncontrolled manner without depleting the complete population of T cells.

As rapidly dividing cells, T cells are susceptible to genetic mutations that lead e.g. to highly aggressive T cell leukemia (TCL). Today, the TCR composition, i.e. the respective Vα chains and Vβ chains of pathogenic T cells can be rapidly identified in patient samples by next generation sequencing. To this end, one may use patient blood or bone marrow samples for TCL or tissue samples that are subject to T cell attack in autoimmune diseases, e.g. central nervous system spinal fluid in MS. A mab which on the one hand would recognize different Vα chains and/or Vβ chains as they occur in e.g. malignant TCL clones but which on the other hand would not be pan-specific would be of therapeutic value as it could be used to treat e.g. TCL in a patient without depleting the complete TCR repertoire. Thereby, the patient remains immunocompetent to respond to pathogen challenges.

Against this background, the application in a further aspect is concerned with the provision of an antibody or binding fragment thereof for use as a medicament.

In particular, the application relates to the provision of an antibody or binding fragment thereof according to the invention for use in the treatment of TCL.

Correspondingly, the application relates to methods of treating T cell leukemia in a human or animal being by administering antibodies and binding fragments thereof according to the invention.

Further, the application relates to an antibody or binding fragment thereof according to the invention in the manufacture of a medicament for the treatment of T cell leukemia.

### FIGURE LEGENDS

FIG. 1:

FIG. 1A: Schematic depiction of the TCR complex on the cell surface containing the TCR α and 3 chains as well as the CD3 complex (chains ε, γ, and δ). The TCR is composed of two different protein chains, α and β, which in turn consist of variable (V) and constant (C) regions. The variable regions of both the TCRα and the β chain contain hypervariable regions (CDR, complementarity determining regions), among which the CDR3 region determines the specific epitope recognition.

FIG. 1B: Modular retroviral TCR expression vector system. The pRAVx (pRBVx) vector system is based on the pMP71 backbone (Schambach A, Wodrich H, Hildinger M, Bohne J, Krausslich H G, Baum C., Mol Ther. 2000 November; 2(5):435-45; Hildinger M, Abel K L, Ostertag W, Baum C., J Virol. 1999 May; 73(5):4083-9.). Each vector contains the murine constant alpha (mCA) or beta constant (mCB) region and one of 45 human AV or 47 human BV regions (hAVx and hBVx). Each vector contains an identical CDR3 region derived from the OT-1-specific T cell clone. The 45 pRAVx are used successively to produce retroviruses (RV). One AV-specific RV is used to transduce recipient T cells, in combination with a second RV encoding a TRBV chain, containing a murine constant beta and human BV region. Likewise, cells expressing a selected human BV region are produced using pRAV encoding a TRAV, containing a murine constant alpha and human AV region with successive pRBVx vectors. 5′LTR, 3′LTR designates the 5′ and 3′ retroviral long terminal repeats.

FIG. 2: Surface TCR expression on the transduced Jurkat cell line and a selected clone. The Jurkat cells were retrovirally transduced and stained for murine beta chain constant region (mCB) surface expression. TCR positive cells were either directly sorted using the FACS Aria cell sorter or manually subcloned by limiting dilution.

FIG. 3: TCR cell libraries. a) The murine BW−/− TCR library upon completion will express 45 different human AV and 47 different human BV TCR regions as shown in light grey (gradient) to represent the human sequence. All other regions of the TCR are shown in dark grey to indicate their mouse origin. The murine cell library is used for immunization. b) Upon retroviral transduction with selected RVs, cells were stained with anti-mCB-specific antibodies. Positive cells were sorted and TCR expression was compared on BW−/− parental cells, recipient BW−/− cells prior to transduction, AV9-transduced BW cells (BW-AV9 cell line) before sorting and the sorted BW-AV9 cell line. c) The complete Jurkat TCR library will express 45 different human AV and 47 different human BV TCR regions. This human Jurkat library will be used for screening. d) Specific primers for the TRAV9 variable region and control primers specific for TRAV1 region were used for PCR amplification using cDNA from the transduced and sorted BW-AV9 cell line. Subsequently, the amplified DNA band was cut out, sequenced and AV9 sequences confirmed by alignment with the AV9 sequence in IMGT database.

FIG. 4: Cross-species screening using BW^{-/-} and Jurkat⁻⁻ cells. BW-TCR transduced cells were used for immunization, however these cells could not be used for hybridoma screening since they bind mouse or rat Ig non-specifically as shown here for the anti-human AV12-2-specific hybridoma supernatant, as well as for the anti-human BV12-3-specific supernatant. Both hybridoma supernatants stain BW^{-/-} cells irrespective of their TCR expression (first row in a and b). In contrast, the same supernatants stain Jurkat^{-/-} cells only when they express the specific AV or BV TCR chain (second row a and b). TCR-transduced BW^{-/-} cells are stably transduced also with CD3-GFP in order to allow TCR expression, accounting for their moderate level of GFP. To distinguish between non-specific and specific TCR binding on TCR-transduced Jurkat^{-/-} cells, a stable Jurkat^{-/-} cell clone transduced to express high levels of GFP and used as a control during hybridoma supernatant screening. The location in the upper left corner of the histogram indicates the very high GFP signal which allows distinction from the lower level of GFP in BW^{-/-} cells. As shown, Jurkat-GFP cells remain unlabeled when tested with supernatant containing either AV- or BV-specific mabs (second row a and b). This is seen by their failure to shift to the left in the presence of supernatant containing AV- or BV-specific mabs.

FIG. 5: Primary screening of pooled hybridoma supernatants including hybridoma clone 15B4 (FIG. 5A) and 5H4 (FIG. 5B). Pooled hybridoma supernatants were screened using a pool of Jurkat cells expressing four different TCRs and 10% GFP-expressing negative control cells. It is expected that about 45% of cells are shifted toward alexa fluor 647 in the TCR-expressing cells, but not in the Jurkat-GFP fraction if a mab specific for an individual BV region is present in the pooled supernatant.

FIG. 6: Secondary screening of single hybridoma supernatants. Hybridoma supernatants of the single plates were screened using a pool of Jurkat cells expressing four different TCRs and 10% GFP-expressing negative control cells. Secondary screening including 15B4 is shown in FIG. 6A. Secondary screening including 5B4 is shown in FIG. 6B.

FIG. 7: Experimental set-up for in vivo depletion of BV-cluster expressing T cells in human ABab TCR transgenic mice with a cluster TCR-specific mab.

FIG. 8: Structures of different TCR constructs to reduce TCR mispairing in transgenic settings. Wild-type TCRs comprise the human constant regions (huCα, huCβ) and one disulfide bond linking the two TCR chains via two cysteine residues (Cys). Cys-mutants comprise an additional disulfide bond, therefore increasing the linkage between modified TCR chains. In murinized constructs the human constant regions are replaced by murine constant regions (muCα) to enhance stable surface expression and preferable pairing. To reduce possible immunogenicity due to foreign mouse segments, the minimal murinized constructs comprise only critical murine amino acids required for improved surface expression and pairing.

FIG. 9: Modular vector system.

FIG. 9A: The TCR constructs are constructed in such a way that each segment or segment variation (variable region, linker sequence comprising CDR3 region and constant C region) as well as any vector backbone (e.g. retro-, lenti-, transposon,-ivtRNA.) can be easily exchanged in a single step procedure. Thereby any type of TCR chain can be generated by exchange of the variable region. The specificity can be switched by the introduction of a desired CDR3 region which can be introduced for example by hybridized oligonucleotides. Moreover, the segments can also be switched between different species versions and modified versions (such as human, murine, cysteine-engineered). FIG. 9B: reconstituted TRAV and TRBV chains can be introduced in the same vector as whole genes divided by P2A sequence. Alternatively, AV-CDR3-J and BV-CDR3-J/D can be introduced in front of a mouse or human constant region that is already incorporated in the vector backbone.

FIG. 10: Vector maps of example vectors having a mouse constant segment. FIG. 10A shows an example of retroviral vector carrying TCR α chain composed of human AV1-1, CDR3 derived for OT1 TCR α chain and mouse alpha constant region. The sequence of this vector is set forth in SEQ ID NO: 204. FIG. 10B shows an example of retroviral vector carrying TCR β chain composed of human BV2, CDR3 derived for OT1 TCR β chain and mouse constant β region. The sequence of this vector is set forth in SEQ ID NO: 205.

FIG. 11: Vector maps of example vectors comprising a human constant region.

FIG. 11A shows an example of a retroviral vector carrying TCR α chain composed of human AV14, CDR3 derived from T1.8 TCR α chain and mouse human constant region. The sequence of this vector is set forth in SEQ ID NO: 208. FIG. 11B shows an example of a retroviral vector carrying TCR β chain composed of human BV27, CDR3 derived from T1.8 TCR β chain and mouse constant β region. The sequence of this vector is set forth in SEQ ID NO: 209.

FIG. 12: Reengineering of an isolated TCR

FIG. 12A: Functional analysis of isolated T cell clone T1.8-3-200. Interferon-gamma (IFN-γ) measurements of co-culture of T cell clone T1.8-3-200 with HLA-matched NY-ESO1-X-(human NY-ESO1 antigen fused to a signal peptide)-loaded APC. FIG. 12B: IMGT sequence analysis of T1.8-3-200 TCRα chain. FIG. 12C: IMGT sequence analysis of T1.8-3-200 TCRβ chain. FIG. 12D: Transgenic function analysis of TCR T1.8-3-200. IFN-γ measurements of co-culture of the T1.8-3-200 TCR-transfected PBL with HLA-matched NY-ESO1-X-loaded APC.

FIG. 13: FIG. 13 A: Induction of ADCC in BV12-3 expressing human leukemia cells by candidate antibodies. Antibodies were incubated at 33.5-66.7 nM with human leukemia cells (Jurkat) expressing the human TCR variable alpha chain 3 and beta chain 12-3 (recognized by the isolated antibodies) and NK cells at different target-to-effector ratios for 6 hrs. FIG. 13 B: Induction of ADCC in BV3-1-expressing human leukemia cells by candidate antibodies. Antibodies were incubated at 33.5-66.7 nM with human leukemia cells (Jurkat) expressing the human TCR variable alpha chain 12-2 and beta chain 3-1 (not recognized by the isolated antibodies) and NK cells at different target-to-effector ratios for 6 hrs.

FIG. 14: In vivo depletion of T cells expressing BV12-3-related TCRs in a humanized TCR mouse model. FIG. 14 A: CD3+ positive PBL of blood samples before treatment. FIG. 14B: FIG. 14 B: CD3+ positive PBL of blood samples on day 4 after treatment; FIG. 14 C: CD3+ positive PBL of blood samples on day 21 after treatment. Antibodies used for staining and depletion treatment are indicated in the legend.

FIG. 15: Measurement of IL-2 and IL-4 during in vivo depletion. Serum concentration of IL-2 and IL-4 0, 2, 6, 12 and 24 hours after antibody treatment. Application of αCD3-AB to mouse #6077 was unsuccessful, therefore no reaction was expected.

FIG. 16: Measurement of IL-10, IL-6, INFγ and TNFα during in vivo depletion. Serum concentration of IL-2 and IL-4 0, 2, 6, 12 and 24 hours after antibody treatment. The legend is shown in FIG. 15. Application of αCD3-AB to mouse #6077 was unsuccessful, therefore no reaction was expected.

FIG. 17: TRBV chains isolated by cluster specific R12 5H4 and R12 15B4 antibodies determined by next generation sequencing. The lymphocytes sorted after staining with R12 5H4 and R12 15B4 were NGS sequenced and BV repertoire analyzed. The identified BV sequences are presented as a percentage of specific TRBV chains. Thus the bars indicate the percentage of the specific TRBV chain with respect to the total amount of TRBV chains within the respective sample (5H4 sorted, 115B4 sorted or whole PBMC respectively). Upon staining with R12 5H4 the sorted population contained TRBV7-2, TRBV7-3, TRBV12-3/TRBV12-4* and TRBV 25-1/TRBV28* (gray bars). The isolated BV repertoire using R12 15B4 is composed of TRBV2, TRBV5-6, TRBV6-5, TRBV7-3, TRBV7-9, TRBV10-3, TRBV12-3/TRBV12-4*, TRBV12-5, TRBV21-1, TRBV6-7/TRBV24-1* and TRBV 25-1/TRBV28* (white bars). These results indicate that these antibodies recognize small similar but distinct clusters. The sequencing of unsorted whole PBMC repertoire served as internal control (black bars). Due to a high sequence homology the chains pairs marked with “*” were recognized as a group.

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of +10%, and preferably of 5%.

Technical terms are used by their common sense. If a specific meaning is conveyed to certain terms, definitions of terms will be given in the following in the context of which the terms are used.

One aspect of the invention refers to an antibody or binding fragment thereof that binds to a fraction of T cell receptor variable alpha (TCR Vα) chains comprising at least two different TCR Vα chains but less than all TCR Vα chains or that binds to a fraction of T cell receptor variable beta (TCR Vβ) chains comprising at least two different TCR Vβ chains but less than all TCR Vβ chains.

The terms “fraction”, “fraction of TCR Vα chains” or “fraction of TCR Vβ chains” as used herein means the specific group of TCR Vα chains or TCR Vβ chains to which the antibody is binding which is smaller than the group of all TCR Vα chains or all TCR Vβ chains, but larger than just one specific TCR Vα chain or TCR Vβ chain.

In other words, an antibody or binding fragment according to the invention does not bind to only to one TCR Vα chain, i.e. type of TCR Vα chain or one TCR Vβ chain, i.e. type of TCR Vβ chain, but binds to several TCR Vα chains, i.e. type of TCR Vα chains or TCR Vβ chains, i.e. type of TCR Vβ chains.

The antibodies or binding fragments of the invention bind to a fraction of TCR Vα chains that is smaller than all functional TCR Vα chains or binds to a fraction of TCR Vβ chains which is smaller than all functional TCR Vβ chains. In other words the antibodies of the invention are not pan-specific antibodies that recognize all TCR Vα chains and/or all TCR Vβ chains, in particular all functional TCR Vα chains or functional TCR Vβ chains.

The terms “functional TCR Vα chains” or “functional TCR Vβ chains” or “functional TCR variable chains” relate to TCR variable chains that are expressed on T cells. That means that this term does not include TCR variable chains that are not expressed, such as pseudogenes, i.e. genes with frameshift mutations or defects in the recombination signal. The annotation whether a TCR variable chain is functional or rather a pseudogene can be found for example in Folch (“The Human T cell Receptor Beta Variable (TRBV) Genes”, Folch Geraldibem Lefranc Maire-Paule, Exp Clin Immunogenet 2000; 17:42-54) or Su et al. (Chen Su and Masatoshi Nei, Mol.-Biol. Evol. 2001; 18(4):505-513). Correspondingly, the term “functional TCR types” refers to TCRs that are composed of TCR variable chains that are expressed on T cells.

In one embodiment, the antibody or binding fragment thereof binds a fraction of TCR Vβ chains comprising at least 3, at least 4, at least 5, at least 6, at least 7, at least 8, at least 9, at least 10, at least 15 or at least 20 different TCR Vβ chains. The invention thus contemplates an antibody or binding fragment thereof which binds to a fraction of TCR Vβ chains comprising at least 3, 4, 5, 6, 7, 8, 9 10, 11, 12, 13, 14, 15, 20, 21, 22, 23, 24, 25, 26, 27, 28, 29, 30, 31, 32, 33, 34, 35, 36, 37, 38, 39 or 40 different TCR Vβ chains. Antibodies or binding fragments thereof which bind larger numbers of different TCR Vβ chains (e.g. 20 different Vβ chains compared to 2 different Vβ chains) are in general of particular interest as these antibodies may e.g. be more broadly usable for TCR-related diseases such as TCL in different patients.

In a specific embodiment, the antibody or binding fragment thereof binds to a fraction of TCR Vβ chains consisting of 3, 4, 5, 6, 7, 8, 9 10, 11, 12, 13, 14, 15, 16, 17, 18, 19, 20, 21, 22, 23, 24, 25, 26, 27, 28, 29 or 30 different TCR Vβ chains selected from the group consisting of TCR Vβ chains of Table 1.

In another embodiment the antibody or binding fragment thereof binds a fraction of TCR Vα chains comprising at least 3, at least 4, at least 5, at least 6, at least 7, at least 8, at least 9, at least 10, at least 15 or at least 20 different TCR Vα chains. The invention thus contemplates an antibody or binding fragment thereof which binds to a fraction of TCR Vα chains comprising at least 3, 4, 5, 6, 7, 8, 9 10, 11, 12, 3, 14, 15, 20, 21, 22, 23, 24, 25, 26, 27, 28, 29, 30, 31, 32, 33, 34, 35, 36, 37, 38, 39 or 40 different TCR Vα chains. Antibodies or binding fragments thereof which bind larger numbers of different TCR Vα chains (e.g. 20 different Vα chains compared to 2 different Vα chains) are in general of particular interest as these antibodies may e.g. be more broadly usable for TCR related diseases such as TCL in different patients.

In a specific embodiment the antibody or binding fragment thereof binds to a fraction of TCR Vα chains consisting of 3, 4, 5, 6, 7, 8, 9 10, 11, 12, 13, 14, 15, 16, 17, 18, 19, 20, 21, 22, 23, 24, 25, 26, 27, 28, 29 or 30 different TCR Vα chains selected from the group consisting of TCR Vα chains of Table 1.

TABLE 1
Table 1 - known functional TCR Vα chains and
TCR Vβ chains. AV stands for variable α
chain and BV stands for variable β chain.

| TCR V class | TCR V type |
|---|---|
| TCR Vβ chains | BV2, BV3-1, BV4-1, BV4-2, BV4-3, |
| | BV5-1, BV5-4, BV5-5, BV5-6, BV5-8, |
| | BV6-1, BV6-2, BV6-4, BV6-5, BV6-6, |
| | BV6-8, BV6-9, BV7-2, BV7-3, BV7-4, |
| | BV7-6, BV 7-7, BV 7-8, BV7-9, BV9, |
| | BV10-1, BV10-2, BV10-3, BV11-1, |
| | BV11-2, BV11-3, BV12-3, BV12-4, |
| | BV12-5, BV13, BV14, BV15, BV16, |
| | BV18, BV19, BV20-1, BV 24-1, BV25-1, |
| | BV27, BV28, BV29-1, BV30 |
| TCR Vα chains | AV1-1, AV1-2, AV2, AV3, AV4, AV5, |
| | AV6, AV7, AV8-1, AV8-2, AV8-3, |
| | AV8-4, AV8-6, AV9-1, AV9-2, AV10, |
| | AV12-1, AV12-2, AV12-3, AV13-1, |
| | AV13-2, AV14/DV4, AV16, AV17, |
| | AV18, AV19, AV20, AV21, AV22, |
| | AV23/DV6, AV24, AV25, AV26-1, |
| | AV26-2, AV27, AV29/DV5, AV30, |
| | AV34, AV35, AV36/DV7, AV38-1, |
| | AV38-2/DV8, AV39, AV40, AV41 |

TABLE 2
TCR Vα chains and TCR Vβ chains with identifiers for
their nucleic acid sequences and amino acid sequences.

| | | nucleic acid | amino acid |
|---|---|---|---|
| Identifier | TCR V type | sequence | sequence |
| AVseg1 | AV1-1 | SEQ ID NO: 8 | SEQ ID NO: 100 |
| AVseg2 | AV1-2 | SEQ ID NO: 9 | SEQ ID NO: 101 |
| AVseg3 | AV2 | SEQ ID NO: 10 | SEQ ID NO: 102 |
| AVseg4 | AV3 | SEQ ID NO: 11 | SEQ ID NO: 103 |
| AVseg5 | AV4 | SEQ ID NO: 12 | SEQ ID NO: 104 |
| AVseg6 | AV5 | SEQ ID NO: 13 | SEQ ID NO: 105 |
| AVseg7 | AV6 | SEQ ID NO: 14 | SEQ ID NO: 106 |
| AVseg8 | AV7 | SEQ ID NO: 15 | SEQ ID NO: 107 |
| AVseg9 | AV8-1 | SEQ ID NO: 16 | SEQ ID NO: 108 |
| AVseg10 | AV8-2 | SEQ ID NO: 17 | SEQ ID NO: 109 |
| AVseg11 | AV8-3 | SEQ ID NO: 18 | SEQ ID NO: 110 |
| AVseg12 | AV8-4 | SEQ ID NO: 19 | SEQ ID NO: 111 |
| AVseg13 | AV8-6 | SEQ ID NO: 20 | SEQ ID NO: 112 |
| AVseg14 | AV9-1 | SEQ ID NO: 21 | SEQ ID NO: 113 |
| AVseg15 | AV9-2 | SEQ ID NO: 22 | SEQ ID NO: 114 |
| AVseg16 | AV10-1 | SEQ ID NO: 23 | SEQ ID NO: 115 |
| AVseg17 | AV12-1 | SEQ ID NO: 24 | SEQ ID NO: 116 |
| AVseg18 | AV12-2 | SEQ ID NO: 25 | SEQ ID NO: 117 |
| AVseg19 | AV12-3 | SEQ ID NO: 26 | SEQ ID NO: 118 |
| AVseg20 | AV13-1 | SEQ ID NO: 27 | SEQ ID NO: 119 |
| AVseg21 | AV13-2 | SEQ ID NO: 28 | SEQ ID NO: 120 |
| AVseg22 | AV14/DV4 | SEQ ID NO: 29 | SEQ ID NO: 121 |
| AVseg23 | AV16 | SEQ ID NO: 30 | SEQ ID NO: 122 |
| AVseg24 | AV17 | SEQ ID NO: 31 | SEQ ID NO: 123 |
| AVseg25 | AV18 | SEQ ID NO: 32 | SEQ ID NO: 124 |
| AVseg26 | AV19 | SEQ ID NO: 33 | SEQ ID NO: 125 |
| AVseg27 | AV20 | SEQ ID NO: 34 | SEQ ID NO: 126 |
| AVseg28 | AV21 | SEQ ID NO: 35 | SEQ ID NO: 127 |
| AVseg29 | AV22 | SEQ ID NO: 36 | SEQ ID NO: 128 |
| AVseg30 | AV23/DV6 | SEQ ID NO: 37 | SEQ ID NO: 129 |
| AVseg31 | AV24 | SEQ ID NO: 38 | SEQ ID NO: 130 |
| AVseg32 | AV25 | SEQ ID NO: 39 | SEQ ID NO: 131 |
| AVseg33 | AV26-1 | SEQ ID NO: 40 | SEQ ID NO: 132 |
| AVseg34 | AV26-2 | SEQ ID NO: 41 | SEQ ID NO: 133 |
| AVseg35 | AV27 | SEQ ID NO: 42 | SEQ ID NO: 134 |
| AVseg36 | AV29/DV5 | SEQ ID NO: 43 | SEQ ID NO: 135 |
| AVseg37 | AV30 | SEQ ID NO: 44 | SEQ ID NO: 136 |
| AVseg38 | AV34 | SEQ ID NO: 45 | SEQ ID NO: 137 |
| AVseg39 | AV35 | SEQ ID NO: 46 | SEQ ID NO: 138 |
| AVseg40 | AV36/DV7 | SEQ ID NO: 47 | SEQ ID NO: 139 |
| AVseg41 | AV38-1 | SEQ ID NO: 48 | SEQ ID NO: 140 |
| AVseg42 | AV38-2/DV8 | SEQ ID NO: 49 | SEQ ID NO: 141 |
| AVseg43 | AV39 | SEQ ID NO: 50 | SEQ ID NO: 142 |
| AVseg44 | AV40 | SEQ ID NO: 51 | SEQ ID NO: 143 |
| AVseg45 | AV41 | SEQ ID NO: 52 | SEQ ID NO: 144 |
| BVseg1 | BV2 | SEQ ID NO: 53 | SEQ ID NO: 145 |
| BVseg2 | BV3-1 | SEQ ID NO: 54 | SEQ ID NO: 146 |
| BVseg3 | BV4-1 | SEQ ID NO: 55 | SEQ ID NO: 147 |
| BVseg4 | BV4-2 | SEQ ID NO: 56 | SEQ ID NO: 148 |
| BVseg5 | BV4-3 | SEQ ID NO: 57 | SEQ ID NO: 149 |
| BVseg6 | BV5-1 | SEQ ID NO: 58 | SEQ ID NO: 150 |
| BVseg7 | BV5-4 | SEQ ID NO: 59 | SEQ ID NO: 151 |
| BVseg8 | BV5-5 | SEQ ID NO: 60 | SEQ ID NO: 152 |
| BVseg9 | BV5-6 | SEQ ID NO: 61 | SEQ ID NO: 153 |
| BVseg10 | BV5-8 | SEQ ID NO: 62 | SEQ ID NO: 154 |
| BVseg11 | BV6-1 | SEQ ID NO: 63 | SEQ ID NO: 155 |
| BVseg12 | BV6-2 | SEQ ID NO: 64 | SEQ ID NO: 156 |
| BVseg13 | BV6-4 | SEQ ID NO: 65 | SEQ ID NO: 157 |
| BVseg14 | BV6-5 | SEQ ID NO: 66 | SEQ ID NO: 158 |
| BVseg15 | BV6-6 | SEQ ID NO: 67 | SEQ ID NO: 159 |
| BVseg16 | BV6-8 | SEQ ID NO: 68 | SEQ ID NO: 160 |
| BVseg17 | BV6-9 | SEQ ID NO: 69 | SEQ ID NO: 161 |
| BVseg18 | BV7-2 | SEQ ID NO: 70 | SEQ ID NO: 162 |
| BVseg19 | BV7-3 | SEQ ID NO: 71 | SEQ ID NO: 163 |
| BVseg20 | BV7-4 | SEQ ID NO: 72 | SEQ ID NO: 164 |
| BVseg21 | BV7-6 | SEQ ID NO: 73 | SEQ ID NO: 165 |
| BVseg22 | BV7-7 | SEQ ID NO: 74 | SEQ ID NO: 166 |
| BVseg23 | BV7-8 | SEQ ID NO: 75 | SEQ ID NO: 167 |
| BVseg24 | BV7-9 | SEQ ID NO: 76 | SEQ ID NO: 168 |
| BVseg25 | BV9 | SEQ ID NO: 77 | SEQ ID NO: 169 |
| BVseg26 | BV10-1 | SEQ ID NO: 78 | SEQ ID NO: 170 |
| BVseg27 | BV10-2 | SEQ ID NO: 79 | SEQ ID NO: 171 |
| BVseg28 | BV10-3 | SEQ ID NO: 80 | SEQ ID NO: 172 |
| BVseg29 | BV11-1 | SEQ ID NO: 81 | SEQ ID NO: 173 |
| BVseg30 | BV11-2 | SEQ ID NO: 82 | SEQ ID NO: 174 |
| BVseg31 | BV11-3 | SEQ ID NO: 83 | SEQ ID NO: 175 |
| BVseg32 | BV12-3 | SEQ ID NO: 84 | SEQ ID NO: 176 |
| BVseg33 | BV12-4 | SEQ ID NO: 85 | SEQ ID NO: 177 |
| BVseg34 | BV12-5 | SEQ ID NO: 86 | SEQ ID NO: 178 |
| BVseg35 | BV13 | SEQ ID NO: 87 | SEQ ID NO: 179 |
| BVseg36 | BV14 | SEQ ID NO: 88 | SEQ ID NO: 180 |
| BVseg37 | BV15 | SEQ ID NO: 89 | SEQ ID NO: 181 |
| BVseg38 | BV16 | SEQ ID NO: 90 | SEQ ID NO: 182 |
| BVseg39 | BV18 | SEQ ID NO: 91 | SEQ ID NO: 183 |
| BVseg40 | BV19 | SEQ ID NO: 92 | SEQ ID NO: 184 |
| BVseg41 | BV20-1 | SEQ ID NO: 93 | SEQ ID NO: 185 |
| BVseg42 | BV24-1 | SEQ ID NO: 94 | SEQ ID NO: 186 |
| BVseg43 | BV25-1 | SEQ ID NO: 95 | SEQ ID NO: 187 |
| BVseg44 | BV27 | SEQ ID NO: 96 | SEQ ID NO: 188 |
| BVseg45 | BV28 | SEQ ID NO: 97 | SEQ ID NO: 189 |
| BVseg46 | BV29-1 | SEQ ID NO: 98 | SEQ ID NO: 190 |
| BVseg47 | BV30 | SEQ ID NO: 99 | SEQ ID NO: 191 |

The nucleotide sequences coding for the variable region of the TCR α and the TCR β chains include leader sequences. During maturation the leader sequence is cleaved off, which means that the protein sequence of the variable region of the TCR α and the TCR β chain is devoid of the leader sequence. The amino acid sequences of the variable regions of the TCR α and the TCR β chains disclosed herein therefore do not contain the leader sequence.

The variable region of the TCR α chain AV1-1 is encoded by the AV segment AVseg1 (SEQ ID No. 8) and has an amino acid sequence of SEQ ID No. 100.

In certain embodiments, the variable AV segments AVseg1 to AVseg45 code for variable TCR α chain regions which are at least 80% identical to the sequences set forth in SEQ ID NO: 100 to SEQ ID NO: 144 and wherein the variable BV segments BVseg 1 to BVseg 47 code for variable TCR β chain regions which are least 80% identical to the sequences set forth in SEQ ID NO: 145 to SEQ ID NO: 191.

In certain embodiments, the variable AV segments AVseg1 to AVseg45 code for variable TCR α chain region which are at least 70%, 75%, 80%, 85%, 90%, 95%, 96%, 97%, 98%, 99% identical to the sequences set forth in SEQ ID NO: 100 to SEQ ID NO: 144 and wherein the variable BV segments BVseg1 to BVseg 47 code for variable TCR β chain regions which are least 70%, 75%, 80%, 85%, 90%, 95%, 96%, 97%, 98%, 99% identical to the sequences set forth in SEQ ID NO: 145 to SEQ ID NO: 199.

In certain embodiments, the variable AV segments AVseg1 to AVseg45 code for variable TCR α chain regions which have sequences set forth in SEQ ID NO: 100 to SEQ ID NO: 144 and wherein the variable BV segments BVseg1 to BVseg47 code for variable TCR β chain regions which have sequences set forth in SEQ ID NO: 145 to SEQ ID NO: 199.

In certain embodiments, the variable AV segments AVseg1 to AVseg45 have sequences which are at 80 identical to the sequences set forth in SEQ ID NO: 8 to SEQ ID NO: 52 and the variable BV segments BVseg1 to BVseg47 segments have sequences which are at least 80% identical to the sequences set forth in SEQ ID NO: 53 to SEQ ID NO: 99.

In certain embodiments, the variable AV segments AVseg1 to AVseg45 have sequences which are at least 70%, 75%, 80%, 85%, 90%, 95%, 96%, 97%, 98%, 99% identical to the sequences set forth in SEQ ID NO: 8 to SEQ ID NO: 52 and the variable BV segments BVseg1 to BVseg47 segments have sequences which are at least 70%, 75%, 80%, 85%, 90%, 95%, 96%, 97%, 98%, 99% identical to the sequences set forth in SEQ ID NO: 53 to SEQ ID NO: 99.

In certain embodiments, the variable AV segments AVseg1 to AVseg45 segments have sequences which are set forth in SEQ ID NO: 8 to SEQ ID NO: 52 and the variable BV segments BVseg1 to BVseg47 segments have sequences which are set forth in SEQ ID NO: 53 to SEQ ID NO: 99.

In one embodiment of the invention the fraction of TCR Vα chains comprises at least two different TCR Vα chains that belong to two different TCR Vα chain subfamilies or wherein the fraction TCR Vβ chains comprises least two different TCR Vβ chains that belong to two different TCR Vβ chain subfamilies.

The term subfamily as used herein refers to conventional gene notation for VB genes. In the nomenclature each gene is denoted by two numbers. The first number represents the subfamily to which the gene belongs; the second indicates the order of discovery of the genes in each subfamily. For example, the variable chains BV6-1, BV6-2, BV6-4, BV6-5, BV6-6, BV6-8, BV6-9 belong to one subfamily.

TCR Vα chains can thus be grouped into 34 subfamilies as shown in Table 3.

TABLE 3
Table 3 - TCR Vα chains grouped into subfamilies,
AV stands for variable α chain.

| Identifier | TCR Vα Subfamily | TCR Vα type |
|---|---|---|
| M1 | FAV1 | AV1-1, AV1-2 |
| M2 | FAV2 | AV2 |
| M3 | FAV3 | AV3 |
| M4 | FAV4 | AV4 |
| M5 | FAV5 | AV5 |
| M6 | FAV6 | AV6 |
| M7 | FAV7 | AV7 |
| M8 | FAV8 | AV8-1, AV8-2, AV8-3, |
| | | AV8-4, AV8-6 |
| M9 | FAV9 | AV9-1, AV9-2 |
| M10 | FAV10 | AV10-1 |
| M11 | FAV12 | AV12-1, AV12-2, |
| | | AV12-3 |
| M12 | FAV13 | AV13-1, 13-2 |
| M13 | FAV14 | AV14/DV4 |
| M14 | FAV16 | AV16 |
| M15 | FAV17 | AV17 |
| M16 | FAV18 | AV18 |
| M17 | FAV19 | AV19 |
| M18 | FAV20 | AV20 |
| M19 | FAV21 | AV21 |
| M20 | FAV22 | AV22 |
| M21 | FAV23 | AV23/DV6 |
| M22 | FAV24 | AV24 |
| M23 | FAV25 | AV25 |
| M24 | FAV26 | AV26-1, AV26-2 |
| M25 | FAV27 | AV27 |
| M26 | FAV29 | AV29/DV5 |
| M27 | FAV30 | AV30 |
| M28 | FAV34 | AV34 |
| M29 | FAV35 | AV35 |
| M30 | FAV36 | AV36/DV7 |
| M31 | FAV38 | AV38-1, AV38-2/DV8 |
| M32 | FAV39 | AV39 |
| M33 | FAV40 | AV40 |
| M34 | FAV41 | AV41 |

TCR Vβ chains can be grouped into 23 subfamilies as shown in Table 4:

TABLE 4
TCR Vβ chains grouped into subfamilies.
BV stands for variable β chain

| Identifier | TCR Vβ Subfamily | TCR Vβ type |
|---|---|---|
| N1 | FBV2 | BV2 |
| N2 | FBV3 | BV3-1 |
| N3 | FBV4 | BV4-1, BV4-2, BV4-3 |
| N4 | FBV5 | BV5-1, BV5-4, BV5-5, |
| | | BV5-6, BV5-8 |
| N5 | FBV6 | BV6-1, BV6-2, BV6-4, |
| | | BV6-5, BV6-6, BV6-8, |
| | | BV6-9 |
| N6 | FBV7 | BV7-2, BV7-3, BV7-4, |
| | | BV7-6, BV7-7, BV7-8, |
| | | BV7-9, |
| N7 | FBV9 | BV9 |
| N8 | FBV10 | BV10-1, BV10-2, BV10-3, |
| N9 | FBV11 | BV11-1, BV11-2, BV11-3 |
| N10 | FBV12 | BV12-3, BV12-4, BV12-5 |
| N11 | FBV13 | BV13 |
| N12 | FBV14 | BV14 |
| N13 | FBV15 | BV15 |
| N14 | FBV16 | BV16 |
| N15 | FBV18 | BV18 |
| N16 | FBV19 | BV19 |
| N17 | FBV20 | BV20-1 |
| N18 | FBV24 | BV24-1 |
| N19 | FBV25 | BV25-1 |
| N20 | FBV27 | BV27 |
| N21 | FBV28 | BV28 |
| N22 | FBV29 | BV29-1 |
| N23 | FBV30 | BV30-1 |

The invention thus contemplates that the fraction of TCR Vα chains comprises at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, or at least 25 different TCR Vα chains that belong to at least 2 different TCR Vα chain subfamilies. The at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, or at least 25 different TCR Vα chains can of course belong also to more than at least 2, such as at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, or at least 24 different TCR Vα chain subfamilies. Antibodies or binding fragments that recognize TCR Vα chains from larger numbers of different TCR Vα chain subfamilies are in general of particular interest as these antibodies may e.g. be more broadly usable for TCR related diseases such as TCL in different patients. Such antibodies or binding fragments thereof may have even broader application than TCR specific antibodies or binding fragments thereof that recognize different TCR Vα chains which all belong to the same subfamily.

The invention correspondingly contemplates that the fraction of TCR Vα chains comprises at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 2 different TCR Vα chain subfamilies.

The invention thus contemplates that the fraction of TCR Vα chains comprises at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 3 different TCR Vα chain subfamilies.

The invention also contemplates that the fraction of TCR Vα chains comprises at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 4 different TCR Vα chain subfamilies.

The invention also contemplates that the fraction of TCR Vα chains comprises at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 5 different TCR Vα chain subfamilies.

The invention also contemplates that the fraction of TCR Vα chains comprises at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 6 different TCR Vα chain subfamilies.

The invention also contemplates that the fraction of TCR Vα chains comprises at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 7 different TCR Vα chain subfamilies.

The invention also contemplates that the fraction of TCR Vα chains comprises at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 8 different TCR Vα chain subfamilies.

The invention also contemplates that the invention the fraction of TCR Vα chains comprises at least 9, at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 9 different TCR Vα chain subfamilies.

The invention also contemplates that the fraction of TCR Vα chains comprises at least 10, at least 11, at least 12, at least 13, at least 14, at least 15, at least 16, at least 17, at least 18, at least 20, at least 21, at least 22, at least 23, at least 24, at least 25 different TCR Vα chains that belong to at least 10 different TCR Vα chain subfamilies.

The invention also contemplates that the fraction of TCR Vα chains comprises at least 11, at least 12, at least 13, at least 14, at least 15, at least 16, at least 17, at least 18, at least 20, at least 21, at least 22, at least 23, at least 24, at least 25 different TCR Vα chains that belong to at least 11 different TCR Vα chain subfamilies.

The invention also contemplates that the fraction of TCR Vα chains comprises at least 12, at least 13, at least 14, at least 15, at least 16, at least 17, at least 18, at least 20, at least 21, at least 22, at least 23, at least 24, at least 25, at least 30, at least 35 different TCR Vα chains that belong to at least 12 different TCR Vα chain subfamilies.

The invention also contemplates that the fraction of TCR Vα chains comprises at least 12, at least 13, at least 14, at least 15, at least 16, at least 17, at least 18, at least 20, at least 21, at least 22, at least 23, at least 24, at least 25, at least 30, at least 35 different TCR Vα chains that belong to at least 15 different TCR Vα chain subfamilies.

The invention also contemplates that the fraction of TCR Vα chains comprises at least 12, at least 13, at least 14, at least 15, at least 16, at least 17, at least 18, at least 20, at least 21, at least 22, at least 23, at least 24, at least 25, at least 30, at least 35 different TCR Vα chains that belong to at least 20 different TCR Vα chain subfamilies.

The invention also contemplates that the fraction of TCR Vα chains comprises at least 12, at least 13, at least 14, at least 15, at least 16, at least 17, at least 18, at least 20, at least 21, at least 22, at least 23, at least 24, at least 25, at least 30, at least 35 different TCR Vα chains that belong to at least 25 different TCR Vα chain subfamilies.

The invention also contemplates that the fraction of TCR Vα chains comprises at least 12, at least 13, at least 14, at least 15, at least 16, at least 17, at least 18, at least 20, at least 21, at least 22, at least 23, at least 24, at least 25, at least 30, at least 35 different TCR Vα chains that belong to at least 30 different TCR Vα chain subfamilies.

The invention thus contemplates that the fraction of TCR Vβ chains comprises at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, or at least 25 different TCR Vβ chains that belong to at least 2 different TCR Vβ chain subfamilies. The least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, or at least 25 different TCR Vβ chains can of course belong also to more than at least 2, such as at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, or at least 24 different TCR Vβ chain subfamilies. Antibodies or binding fragments that recognize TCR Vβ chains from larger numbers of different TCR Vβ chain subfamilies are in general of particular interest as these antibodies may e.g. be more broadly usable for TCR-related diseases such as TCL in different patients. Such antibodies or binding fragments thereof may have even broader application than cluster TCR-specific antibodies or binding fragments thereof that recognize different TCR Vβ chains which all belong to the same subfamily.

The invention correspondingly contemplates that the fraction of TCR Vβ chains comprises at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 2 different TCR Vβ chain subfamilies.

The invention thus contemplates that the fraction of TCR Vβ chains comprises at least 3, at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 3 different TCR Vβ chain subfamilies.

The invention also contemplates that the fraction of TCR Vβ chains comprises at least 4, at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 4 different TCR Vβ chain subfamilies.

The invention also contemplates that the fraction of TCR Vβ chains comprises at least 5, at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 5 different TCR Vβ chain subfamilies.

The invention also contemplates that the fraction of TCR Vβ chains comprises at least 6, at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 6 different TCR Vβ chain subfamilies.

The invention also contemplates that the fraction of TCR Vβ chains comprises at least 7, at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 7 different TCR Vβ chain subfamilies.

The invention also contemplates that the fraction of TCR Vβ chains comprises at least 8, at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 8 different TCR Vβ chain subfamilies.

The invention also contemplates that the invention the fraction of TCR Vβ chains comprises at least 9, at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 9 different TCR Vβ chain subfamilies.

The invention also contemplates that the fraction of TCR Vβ chains comprises at least 10, at least 11, at least 12, at least 13, at least 14, at least 15, at least 16, at least 17, at least 18, at least 20, at least 21, at least 22, at least 23, at least 24, at least 25 different TCR Vβ chains that belong to at least 10 different TCR Vβ chain subfamilies.

The invention also contemplates that the fraction of TCR Vβ chains comprises at least 11, at least 12, at least 13, at least 14, at least 15, at least 16, at least 17, at least 18, at least 20, at least 21, at least 22, at least 23, at least 24, at least 25 different TCR Vβ chains that belong to at least 11 different TCR Vβ chain subfamilies.

The invention also contemplates that the fraction of TCR Vβ chains comprises at least 12, at least 13, at least 14, at least 15, at least 16, at least 17, at least 18, at least 20, at least 21, at least 22, at least 23, at least 24, at least 25 different TCR Vβ chains that belong to at least 12 different TCR Vβ chain subfamilies.

The invention also contemplates that the fraction of TCR Vβ chains comprises at least 12, at least 13, at least 14, at least 15, at least 16, at least 17, at least 18, at least 20, at least 21, at least 22, at least 23, at least 24, at least 25 different TCR Vβ chains that belong to at least 15 different TCR Vβ chain subfamilies.

The invention also contemplates that the fraction of TCR Vβ chains comprises at least 12, at least 13, at least 14, at least 15, at least 16, at least 17, at least 18, at least 20, at least 21, at least 22, at least 23, at least 24, at least 25 different TCR Vβ chains that belong to at least 18 different TCR Vβ chain subfamilies.

The invention also contemplates that the fraction of TCR Vβ chains comprises at least 12, at least 13, at least 14, at least 15, at least 16, at least 17, at least 18, at least 20, at least 21, at least 22, at least 23, at least 24, at least 25 different TCR Vβ chains that belong to at least 21 different TCR Vβ chain subfamilies.

Table 5 shows which groups of different TCR Vβ chains may be recognized by cluster TCR-specific antibodies or binding fragments thereof.

TABLE 5
TCR Vβ chain types groups recognized
by antibodies according to the invention

| TCR Vβ | |
|---|---|
| type group no. | TCR Vβ type |
| T-1 | BV6-2, BV6-3, BV6-4, BV6-5, BV6-6, BV12-3, |
| | BV12-5, BV14, BV18, BV20-1, BV21-1, BV24-1, |
| | BV29-1 |
| T-2 | BV6-4, BV6-5, BV6-6, BV12-3, BV12-5, BV14, |
| | BV18, BV20-1, BV21-1, BV24-1, BV29-1 |
| T-3 | BV6-4, BV6-5, BV6-5, BV12-3, BV14, BV24-1, |
| | BV29-1, |
| T-4 | BV12-3, BV12-5, BV14, BV24-1, BV29-1 |
| T-5 | BV12-3, BV12-5, BV14, BV18, BV24-1, BV29-1 |
| T-6 | BV12-3, BV14, BV18, BV24-1, BV29-1 |
| T-7 | BV5-6, BV12-3, BV12-5, BV13, BV14, BV15, BV18, |
| | BV19, BV24-1, BV25-1 BV29-1 |
| T-8 | BV12-3, BV12-5, BV14, BV18 |
| T-9 | BV6-1, BV6-2, BV6-3, BV6-4, BV6-5, BV6-6, BV6-8, |
| | BV6-9, BV10-1, BV10-2, BV10-3, BV19, BV25-1, |
| | BV27, BV24-1, BV28, BV29-1 |
| T-10 | BV6.1, BV6.2, BV6.3, BV6.5, BV10.3, BV19, BV27, |
| | BV28, BV29-1 |
| T-11 | BV6-1, BV19, BV27, BV28, BV29-1 |
| T-12 | BV11-1, BV11-2, BV11-3, BV12-3, BV12-4, BV12-5, |
| | BV14, BV2, BV4-1, BV4-2, BV4-3, BV7-2, BV7-3, |
| | BV7-4, BV7-6, BV7-7, BV7-8, BV7-9, BV5-1, BV5-4, |
| | BV5-5, BV5-6, BV5-8, BV13, BV16, BV18, BV20-1, |
| | BV9-1, BV3-1, BV15, BV30 |
| T-13 | BV12-3, BV4-2, BV7-3, BV7-6, BV5-1, BV20-1, |
| | BV9, BV3-1, BV2- |
| T-14 | BV12-3, BV2, BV4-2, BV5-1, BV20-1, BV9, BV3-1 |
| T-15 | BV12-3, BV2, BV5-1, BV20-1, BV9 |
| T-16 | BV19, BV29-1, BV5-1, BV20-1 |
| T-17 | BV12-3, BV14, BV24-1, BV29-1 |
| T-18 | BV12-3, BV14-, BV24-1 |
| T-19 | BV6-1, BV19, BV29-1 |
| T-20 | BV19, BV29-1 |
| T-21 | BV5-1, BV20-1 |
| T-22 | BV2, BV5-6, BV6-5, BV7-3, BV7-9, BV10-3, BV12-3, |
| | BV12-4, BV12-5, BV21-1, BV6-7, BV24-1, BV25-1, |
| | BV28 |
| T-23 | BV12-3, BV12-4, BV21-1, BV6-7, BV24-1, BV25-1, |
| | BV28 |
| T24 | BV7-2, BV7-3, BV12-3, BV12-4, BV25-1, BV28 |
| T25 | BV7-2, BV12-3, BV12-4 |

The invention thus contemplates that the fraction of TCR Vβ chains comprises at least two different TCR Vβ chains selected from one of the groups defined in lines T-1 to T-25 of Table 5.

The invention further contemplates that the fraction of TCR Vβ chains comprises at least 3 different TCR Vβ chains selected from one of the groups defined in lines T1 to T-19 and T-22 to T-25 of Table 5.

The invention further contemplates that the fraction of TCR Vβ chains comprises at least 4 different TCR Vβ chains selected from one of the groups defined in lines T-1 to T-17 and T22 to T24 of Table 5.

The invention further contemplates that the fraction of TCR Vβ chains comprises at least 5 different TCR Vβ chains selected from one of the groups defined in lines T-1 to T-15 and T22 to T24 of Table 5.

The invention further contemplates that the fraction of TCR Vβ chains comprises at least two different TCR Vβ chains selected from one of the groups as defined in lines T-1 to T-25 of Table 5 that belong to at least 2 different TCR Vβ chain subfamilies.

The invention further contemplates that the fraction of TCR Vβ chains comprises at least 3 different TCR Vβ chains selected from one of the groups as defined in lines T-1 to T-19 and T22 to T24 of Table 5 that belong to at least 2 different TCR Vβ chain subfamilies.

The invention further contemplates that the fraction of TCR Vβ chains comprises at least 4 different TCR Vβ chains selected from one of the groups defined in lines T-1 to T-17 and T22 to T24 of Table 5 that belong to at least 2 different TCR Vβ chain subfamilies.

The invention further contemplates that the fraction of TCR Vβ chains comprises at least 5 different TCR Vβ chains selected from one of the groups defined in lines T-1 to T-15 and T22 to T24 of Table 5 that belong to at least 2 different TCR Vβ chain subfamilies.

The invention further contemplates that the fraction of TCR Vβ chains comprises at least 3 different TCR Vβ chains selected from one of the groups as defined in lines T-1 to T-19 of Table 5 that belong to at least 3 different TCR Vβ chain subfamilies.

The invention further contemplates that the fraction of TCR Vβ chains comprises at least 4 different TCR Vβ chains selected from one of the groups defined in lines T-1 to T-17 of Table 5 that belong to at least 3 different TCR Vβ chain subfamilies.

The invention further contemplates that the fraction of TCR Vβ chains comprises at least 5 different TCR Vβ chains selected from one of the groups defined in lines T-1 to T-15 of Table 5 that belong to at least 3 different TCR Vβ chain subfamilies.

The invention further contemplates that the fraction of TCR Vβ chains comprises at least 4 different TCR Vβ chains selected from one of the groups defined in lines T-1 to T-17 of Table 5 that belong to at least 4 different TCR Vβ chain subfamilies.

The invention further contemplates that the fraction of TCR Vβ chains comprises at least 5 different TCR Vβ chains selected from one of the groups defined in lines T-1 to T-15 of Table 5 that belong to at least 5 different TCR Vβ chain subfamilies.

Table 6 shows to which groups of subfamilies the TCR Vβ chains may belong, that are recognized by cluster TCR-specific antibodies or binding fragments thereof.

TABLE 6
TCR Vβ chain subfamily groups recognized
by antibodies according to the invention

| TCR Vβ | |
|---|---|
| subfamily | |
| group no. | TCR Vβ subfamily |
| F-1 | FBV6, FBV12, FBV14, FBV18, FBV20, FBV21, |
| | FBV24, FBV29 |
| F-2 | FBV6, FBV12, FBV14, FBV24, FBV29, |
| F-3 | FBV12, FBV14, FBV24, FBV29 |
| F-4 | FBV12, FBV14, FBV18, FBV20, FBV24, FBV29 |
| F-5 | FBV12, FBV14, FBV18, FBV24, FBV29 |
| F-6 | FBV5, FBV12, FBV13, FBV14, FBV15, FBV18, |
| | FBV19, FBV23, FBV24, FBV25, FBV29 |
| F-7 | FBV6, FBV10, FBV19, FBV25, FBV24, FBV27, |
| | FBV28, FBV29 |
| F-8 | FBV6, FBV10, FBV19, FBV27, FBV28, FBV29 |
| F-9 | FBV6, FBV19, FBV27, FBV28, FBV29 |
| F-10 | FBV2, FBV3, FBV4, FBV5, FBV7, FBV9, FBV11, |
| | FBV12, FBV14, FBV13, FBV15, FBV16, FBV18, |
| | FBV20, FBV30 |
| F-11 | FBV2, FBV3, FBV4, FBV5, FBV7, FBV9, FBV12, |
| | FBV20 |
| F-12 | FBV2, FBV3, FBV4, FBV5, FBV9, FBV12, FBV20 |
| F-13 | FBV2, FBV5, FBV9, FBV12, FBV20 |
| F-14 | FBV5, FBV19, FBV20, FBV29, |
| F-15 | FBV12, FBV14, FBV24, FBV29 |
| F-16 | FBV12, FBV14, FBV24 |
| F-17 | FBV6, FBV19, FBV29 |
| F-18 | FBV12, FBV14, FBV18 |
| F-19 | FBV19, FBV29 |
| F-20 | FBV5, FBV20 |
| F-21 | FBV2, FBV5, FBV6, FBV7, FBV10, FBV12, FBV21, |
| | FBV24, FBV25, BV28 |
| F-22 | FBV6, FBV12, FBV21, FBV24, FBV25, FBV28 |
| F-23 | FBV 7, FBV12, FBV 15, |
| F-24 | FBV2, FBV5, FBV6, FBV7, FBV10, FBV12, FBV21, |
| | FBV24, FBV25, FBV28, |
| F-25 | FBV12, FBV21, FBV6, FBV24, FBV25, FBV28 |
| F-26 | FBV12, FBV 7, FBV 15, |

The invention thus contemplates that the fraction of TCR Vβ chains comprises at least two different TCR Vβ chains that belong to at least two different TCR Vβ chain subfamilies as defined in lines F-1 to F-26 of Table 6.

The invention further contemplates that the fraction of TCR Vβ chains comprises at least 3 different TCR Vβ chains that belong to at least 3 different TCR Vβ chain subfamilies as defined in lines F-1 to F-18 and lines F-21 to 26 of Table 6.

The invention further contemplates that the fraction of TCR Vβ chains comprises at least 4 different TCR Vβ chains that belong to at least 4 different TCR Vβ chain subfamilies as defined in lines F-1 to F-15 of Table 6.

The invention further contemplates that the fraction of TCR Vβ chains comprises at least 5 different TCR Vβ chains that belong to at least 5 different TCR Vβ chain subfamilies as defined in lines F-1 to F-13 of Table 6.

The invention further contemplates that the fraction of TCR Vβ chains comprises at least 5, at least 6, at least 7, at least 8, at least 9, at least 10 different TCR Vβ chains that belong to at least 5 different TCR Vβ chain subfamilies as defined in lines F-1 to F-13 of Table 6.

In a specific embodiment of the invention the fraction of TCR Vβ chains comprises at least two different TCR Vβ chains that belong to at least two different TCR Vβ chain subfamilies as defined in lines F-21 to F-26 of Table 6.

If it is stated that an antibody or fragment thereof binds to a variable TCR Vα chains or TCR Vβ chains, this means that the antibody or fragments thereof binds specifically to said variable chains, i.e. binds the variable chain with greater affinity than other variable chains.

For example, an antibody or fragment is specific for its cognate antigen when the variable regions of the antibody or fragment recognize and bind the cognate antigen with a detectable preference distinguishing the antigen from other known polypeptides of similar but not identical sequence by virtue of measurable differences in binding affinity. It will be understood that specific antibodies and fragments may also interact with other proteins (for example, S. aureus protein A or other antibodies in ELISA techniques) through interactions with sequences outside the variable region of the antibodies, and in particular, in the constant region of the antibody or fragment. Screening assays to determine binding specificity of an antibody are well known and routinely practiced in the art. For a comprehensive discussion of such assays (see e.g. 4. Harlow et al. (Eds), Antibodies A Laboratory Manual; Cold Spring Harbor Laboratory; Cold Spring Harbor, N.Y. (1988), Chapter 6).

The antibodies and binding fragments thereof as they are used in the context of the present invention may be preferably monoclonal and more preferably monoclonal chimeric, humanized or human antibodies. A particularly preferred aspect which applies to all embodiments described herein relates to monoclonal humanized antibodies or binding fragments thereof.

The antibodies can be of different subtypes such as of the IgG or IgM class. Antibodies of the IgG class are of particular interest.

Antibodies or binding fragments as described herein are capable to deplete subpopulations of T cells. This means that only a subpopulation of T cells is depleted while the remaining populations are still present after the depletion.

In particular antibodies or binding fragments as described herein are capable to deplete a specific subpopulation of T cells. This means that the antibodies of the present invention deplete a subpopulation of T cells that expresses at least two different TCR Vα chains to which the antibody is binding or deplete a subpopulation of T cells that express at least two different TCR Vβ chains to which the antibody is binding. The remaining T cells do not express the at least two different TCR Vα chains to which the antibody is binding or do not express the at least two different TCR Vβ chains to which the antibody is binding. By binding not only one but several different types of TCR Vα chains or several different types of TCR Vβ permits the specific depletion of different T cells in a larger population of T cells with a single antibody.

These properties of the antibodies or binding fragments described herein may thus allow specifically depleting a subpopulation of T cells which contains aberrant T cells while the remaining T cells not containing aberrant T cells stay intact. The antibodies or binding fragments as described herein may therefore be used as a therapeutic agent, in particular for T-cell related malignancies such as TCL.

Given that the antibodies or binding fragments as described herein can recognize fractions of TCRs from e.g. different subfamilies, this may allow for different malignancies involving aberrant T cells being cured with a limited set of antibodies or binding fragments thereof or even with a single antibody or binding fragment thereof.

Moreover, in conditions that are linked to the aberration of several different T cell types antibodies or binding fragments as described herein can target the different T cell types at once and it is not necessary to target each individual T cell type with a separate specific antibody or binding fragment. Dependent on the combination of the aberrant T cell types, e.g. only one or a combination of e.g. two or three different antibodies is necessary in order to deplete a population of aberrant T cells that comprises a larger number of different T cell types, such as 3 to 20 different T cell types.

Moreover, antibody or binding fragments as described herein may not induce the release of proinflammartory cytokines in the form of a cytokine storm when being used for therapeutic purposes.

The present invention therefore also relates to antibodies or binding fragments thereof as described herein for use as a medicament.

In particular, the application relates to the provision of an antibody or binding fragment thereof according to the invention for use in the treatment of T cell leukemia.

Correspondingly, the application relates to methods of treating T cell leukemia in a human or animal being by administering antibodies and binding fragments thereof according to the invention.

Further, the application relates to an antibody or binding fragment thereof according to the invention in the manufacture of a medicament for the treatment of T cell leukemia.

Turning to more specific aspects a preferred embodiment relates to antibodies or binding fragments thereof which bind to a fraction comprising at least two different TCR Vβ chains. In an even more preferred embodiment the fraction comprises at least two different TCR Vβ chains that belong to different TCR Vβ chain subfamilies. Such antibodies or binding fragments may be used to deplete a subpopulation of T cells expressing at least two different TCR Vβ chains to which the antibody is binding.

Such antibodies or binding fragments thereof may comprise a variable heavy chain and/or a variable light chain of the exemplary antibody 15B4 a variable heavy chain and/or a variable light chain having at least 80% sequence identity with the variable heavy chain and/or variable light chain of the exemplary antibody 15B4. 15B4 is an antibody that was identified in the experimental sections as binding to the human BV12. This sequence may therefore be used to obtain antibodies with similar properties as 15B4 by changing this sequence.

Other contemplated exemplary antibodies or binding fragments thereof may thus comprise the complementarity determining regions (CDRs) of the exemplary antibody 15B4 within their variable heavy chain and/or variable light chain. Such antibodies may also comprise CDRs within their variable heavy chain and/or variable light chain having at least 80% sequence identity with the CDRs of the exemplary antibody 15B4.

The heavy chain of 15B4 is e.g. encoded by SEQ ID No. 223. The light chain of 15B4 is e.g. encoded by SEQ ID No. 222. The heavy chain of 15B4 has thus the amino acid sequence as set out in SEQ ID No: 221. The light chain of 15B4 has thus the amino acid sequence as set out in SEQ ID No: 220.

The variable heavy chain of 15B4 has an amino acid sequence of SEQ ID No.219. The variable light chain of 15B4 has an amino acid sequence of SEQ ID No. 218. As regards the variable heavy chain of 15B4, the CDR1 has an amino acid sequence of SEQ ID No. 215, the CDR2 has an amino acid sequence of SEQ ID No. 216 and the CDR3 has an amino acid sequence of SEQ ID No. 217 As regards the variable light chain of 15B4, the CDR1 has an amino acid sequence of SEQ ID No. 212, the CDR2 has the amino acid sequence “RAS” and the CDR3 has an amino acid sequence of SEQ ID No. 214.

One embodiment refers to the humanized version of 15B4:

The heavy chain of the humanized version of 15B4 is e.g. encoded by SEQ ID No. 304. The light chain of the humanized version of 15B4 is e.g. encoded by SEQ ID No. 303. The heavy chain of the humanized version of 15B4 thus has an amino acid sequence of SEQ ID No. 300. The light chain of the humanized version of 15B4 thus has an amino acid sequence of SEQ ID No. 299. The heavy chain of the humanized version of 15B4 including the signal peptide (which is cleaved of in the mature protein) has an amino acid sequence of SEQ ID No. 302. The light chain of the humanized version of 15B4 including the signal peptide (which is cleaved of in the mature protein) has an amino acid sequence of SEQ ID No. 301.

The variable heavy chain of the humanized version of 15B4 has an amino acid sequence of SEQ ID No. 298. The variable light chain of the humanized version of 15B4 has an amino acid sequence of SEQ ID No. 297. As regards the variable heavy chain of the humanized version of 15B4, the CDR1 has an amino acid sequence of SEQ ID No. 294, the CDR2 has the amino acid sequence of SEQ ID No. 295 and the CDR3 has an amino acid sequence of SEQ ID No. 296. As regards the variable light chain of the humanized version of 15B4, the CDR1 has an amino acid sequence of SEQ ID No. 292, the CDR2 has the amino acid sequence “RAS” and the CDR3 has an amino acid sequence of SEQ ID No. 293.

Another embodiment refers to the chimeric version of 15B4:

The heavy chain of the chimeric version of 15B4 is e.g. encoded by SEQ ID No. 291. The light chain of the chimeric version of 15B4 is e.g. encoded by SEQ ID No. 290. The heavy chain of the chimeric version of 15B4 thus has an amino acid sequence of SEQ ID No. 287. The light chain of the chimeric version of 15B4 thus has an amino acid sequence of SEQ ID No. 286. The heavy chain of the chimeric version of 15B4 including the signal peptide (which is cleaved off in the mature protein) has an amino acid sequence of SEQ ID No. 289. The light chain of the chimeric version of 15B4 including the signal peptide (which is cleaved off in the mature protein) has an amino acid sequence of SEQ ID No. 288.

The variable heavy chain of the chimeric version of 15B4 has an amino acid sequence of SEQ ID No. 285. The variable light chain of the chimeric version of 15B4 has an amino acid sequence of SEQ ID No. 284. As regards the variable heavy chain of the chimeric version of 15B4, the CDR1 has an amino acid sequence of SEQ ID No. 281, the CDR2 has the amino acid sequence of SEQ ID No. 282 and the CDR3 has an amino acid sequence of SEQ ID No. 283. As regards the variable light chain of the chimeric version of 15B4, the CDR1 has an amino acid sequence of SEQ ID No. 279, the CDR2 has the amino acid sequence “RAS” and the CDR3 has an amino acid sequence of SEQ ID No. 280.

Such antibodies or binding fragments thereof may comprise a variable heavy chain and/or a variable light chain of the exemplary antibody 5H4, a variable heavy chain and/or a variable light chain having at least 80% sequence identity with the variable heavy chain and/or variable light chain of the exemplary antibody 5H4. 5H4 is an antibody that was identified in the experimental sections as binding to the human BV12. This sequence may therefore be used to obtain antibodies with similar properties as 5H4 by changing this sequence.

Other contemplated exemplary antibodies or binding fragments thereof may thus comprise the complementarity determining regions (CDRs) of the exemplary antibody 5H4 within their variable heavy chain and/or variable light chain. Such antibodies may also comprise CDRs within their variable heavy chain and/or variable light chain having at least 80% sequence identity with the CDRs of the exemplary antibody 5H4.

The heavy chain of 5H4 is e.g. encoded by SEQ ID No. 237. The light chain of 5H4 is e.g. encoded by SEQ ID No. 236. The heavy chain of 5H4 thus has an amino acid sequence of SEQ ID No. 235. The light chain of 5H4 thus has an amino acid sequence of SEQ ID No. 234.

The variable heavy chain of 5H4 has an amino acid sequence of SEQ ID No. 233. The variable light chain of 5H4 has an amino acid sequence of SEQ ID No. 232. As regards the variable heavy chain of 5H4, the CDR1 has an amino acid sequence of SEQ ID No. 229, the CDR2 has an amino acid sequence of SEQ ID No. 230 and the CDR3 has an amino acid sequence of SEQ ID No. 231. As regards the variable light chain of 5H4, the CDR1 has an amino acid sequence of SEQ ID No. 226, the CDR2 has the amino acid sequence “RAS” and the CDR3 has an amino acid sequence of SEQ ID No. 228.

One embodiment refers to the humanized version of 5H4:

The heavy chain of the humanized version of 5H4 is e.g. encoded by SEQ ID No. 265. The light chain of the humanized version of 5H4 is e.g. encoded by SEQ ID No. 264. The heavy chain of the humanized version of 5H4 thus has an amino acid sequence of SEQ ID No. 261. The light chain of the humanized version of 5H4 thus has an amino acid sequence of SEQ ID No. 260. The heavy chain of the humanized version of 5H4 including the signal peptide (which is cleaved off in the mature protein) has an amino acid sequence of SEQ ID No. 263. The light chain of the humanized version of 5H4 including the signal peptide (which is cleaved off in the mature protein) has an amino acid sequence of SEQ ID No. 262.

The variable heavy chain of the humanized version of 5H4 has an amino acid sequence of SEQ ID No. 259. The variable light chain of the humanized version of 5H4 has an amino acid sequence of SEQ ID No. 258. As regards the variable heavy chain of the humanized version of 5H4, the CDR1 has an amino acid sequence of SEQ ID No. 255, the CDR2 has an amino acid sequence of SEQ ID No. 256 and the CDR3 has an amino acid sequence of SEQ ID No. 257. As regards the variable light chain of the humanized version of 5H4, the CDR1 has an amino acid sequence of SEQ ID No. 253, the CDR2 has the amino acid sequence “RAS” and the CDR3 has an amino acid sequence of SEQ ID No. 254.

One embodiment refers to the chimeric version of 5H4:

The heavy chain of the chimeric version of 5H4 is e.g. encoded by SEQ ID No. 278. The light chain of the chimeric version of 5H4 is e.g. encoded by SEQ ID No. 277. The heavy chain of the chimeric version of 5H4 thus has an amino acid sequence of SEQ ID No. 274. The light chain of the chimeric version of 5H4 thus has an amino acid sequence of SEQ ID No. 273. The heavy chain of the chimeric version of 5H4 including the signal peptide (which is cleaved off in the mature protein) has an amino acid sequence of SEQ ID No. 276. The light chain of the chimeric version of 5H4 including the signal peptide (which is cleaved off in the mature protein) has an amino acid sequence of SEQ ID No. 275.

The variable heavy chain of the chimeric version of 5H4 has an amino acid sequence of SEQ ID No. 272. The variable light chain of the chimeric version of 5H4 has an amino acid sequence of SEQ ID No. 271. As regards the variable heavy chain of the chimeric version of 5H4, the CDR1 has an amino acid sequence of SEQ ID No. 268, the CDR2 has an amino acid sequence of SEQ ID No. 269 and the CDR3 has an amino acid sequence of SEQ ID No. 270. As regards the variable light chain of the chimeric version of 5H4, the CDR1 has an amino acid sequence of SEQ ID No. 266, the CDR2 has the amino acid sequence “RAS” and the CDR3 has an amino acid sequence of SEQ ID No. 267.

Such antibodies or binding fragments thereof may comprise a variable heavy chain and/or a variable light chain of the humanized or chimeric antibody 5H4, a variable heavy chain and/or a variable light chain having at least 80% sequence identity with the variable heavy chain and/or variable light chain of the humanized or chimeric antibody 5H4.

Other contemplated exemplary antibodies or binding fragments thereof may thus comprise the complementarity determining regions (CDRs) of the humanized version of 5H4 within their variable heavy chain and/or variable light chain. Such antibodies may also comprise CDRs within their variable heavy chain and/or variable light chain having at least 80% sequence identity with the CDRs of the humanized version of 5H4.

Further contemplated exemplary antibodies or binding fragments thereof may thus comprise the complementarity determining regions (CDRs) of the chimeric version of 5H4 within their variable heavy chain and/or variable light chain. Such antibodies may also comprise CDRs within their variable heavy chain and/or variable light chain having at least 80% sequence identity with the CDRs of the chimeric version of 5H4.

Preferably, in all these embodiments the sequence identity is at least about 85%, more preferably at least about 90%, even more preferably at least about 95% and most preferably at least about 98% or about 99%. Sequence identity may be determined over the whole length of the respective sequences.

The determination of percent identity between two sequences is preferably accomplished using the mathematical algorithm of Karlin and Altschul (1993) Proc. Natl. Acad. Sci USA 90: 5873-5877. Such an algorithm is incorporated into the BLASTn and BLASTp programs of Altschul et al. (1990) J. Mol. Biol., 215: 403-410 (see references) available at NCBI (http://www.ncbi.nlm.nih.gov/blast/Blast.cge).

The determination of percent identity is performed with the standard parameters of the BLASTn and BLASTp programs.

BLAST polynucleotide searches are performed with the BLASTn program.

For the general parameters, the “Max Target Sequences” box may be set to 100, the “Short queries” box may be ticked, the “Expect threshold” box may be set to 10 and the “Word Size” box may be set to 28. For the scoring parameters the “Match/mismatch Scores” may be set to 1, −2 and the “Gap Costs” box may be set to linear. For the Filters and Masking parameters, the “Low complexity regions” box may not be ticked, the “Species-specific repeats” box may not be ticked, the “Mask for lookup table only” box may be ticked, the “Mask lower case letters” box may not be ticked.

BLAST protein searches are performed with the BLASTp program. For the general parameters, the “Max Target Sequences” box may be set to 100, the “Short queries” box may be ticked, the “Expect threshold” box may be set to 10 and the “Word Size” box may be set to “3”. For the scoring parameters the “Matrix” box may be set to “BLOSUM62”, the “Gap Costs” Box may be set to “Existence: 11 Extension:1”, the “Compositional adjustments” box may be set to “Conditional compositional score matrix adjustment”. For the Filters and Masking parameters the “Low complexity regions” box may not be ticked, the “Mask for lookup table only” box may not be ticked and the “Mask lower case letters” box may not be ticked.

The term “CDR” refers to the complementarity determining region or hypervariable region amino acid residues of an antibody that participate in or are responsible for antigen-binding. The CDRs as described herein are defined according to the international ImMunoGeneTics information System® (LaFranc, et al. 2005. Nucl Acids Res. 33:D593-D597) and as described in (Lefranc et al. Dev. Comparat. Immunol. 27:55-77, 2003).

The above-mentioned CDRs of a light and heavy chain variable region may be embedded in human sequences of framework and constant regions derived from other human antibodies, particularly if such sequences have been shown to be effective in antibody dependent cell mediated cytotoxicity (ADCC). In this context, one may e.g. use the human constant and framework sequences of humanized therapeutic antibodies that have been successfully used for therapeutic applications. The above-mentioned CDRs of a light and heavy chain variable region are preferably incorporated into the framework and constant regions of such humanized antibodies of the human IgG class.

Further, the above-mentioned CDRs of a light and heavy chain variable region may be embedded in essentially human sequences for framework and constant regions. However, particularly the framework regions, but also the constant regions may comprise amino acids as they are e.g. typically found in mouse antibodies which are known to enhance antigen binding and/or e.g. ADCC (see e.g. European patent application EP 0 451 216). Preferably these antibodies are of the IgG class.

In the following several methodologies are described which have been developed for reduction of immunogenicity of non-human derived antibodies, like chimerization or humanization. These approaches may also be applied to other antibodies that can be identified using e.g. the immunization and screening approaches which are described in the experiments hereinafter. They may thus be applied to antibodies and binding fragments thereof that recognize other human Vβ chains than human BV12 or that recognize human Vα chains.

During humanization, all amino acids which are not essential for proper antibody folding or antigen recognition are exchanged with amino acids from the human antibody counterpart. Several methods for mab humanization are developed including traditional CDR grafting or more novel approaches which involve computer modeling and bioinformatics analysis. Humanization of the heavy and light chains of CL 1 was performed using the CDR grafting method (see e.g. Desmet et al. in Kontermann and Dübel (eds.) Antibody Engineering Vol. 1, p. 341ff; Bernett et al. J. Mol. Biol. (2010) 396, 1474-1490). The heavy and light chain variable framework regions can be derived from the same or different human antibody sequences. The human antibody sequences can be the sequences of naturally occurring human antibodies. Human heavy and light chain variable framework regions are listed e.g. in Lefranc, M.-P., Current Protocols in Immunology (2000)—Appendix IP A.1P.1-A.1P.37 and are accessible via IMGT, the international ImMunoGeneTics information System® (http://imgt.cines.fr). A humanized BV cluster mab IGK chain sequence of 15B4 can be prepared based on human IGKV7-3*01(P), IGKV3-11*01, IGKV3-NL5*01, IGKV3D-7*01, IGKV3-NL1*01 and rat 15B4 IGVK chain as indicated in SEQ ID NO: 224.

A humanized BV cluster mab IGH chain sequence of 15B4 may thus be prepared based on human IGHV1-f*0, IGHV1-24*01, IGHJ6*01, IGHD3-10*01 and rat 15B4 IGVH chain as indicated in SEQ ID NO: 225.

It is therefore to be understood that 15B4 and 5H4 serve not only as an example of antibodies or binding fragments thereof which recognize human Vβ chains other than human VB12 but also as an example of antibodies or binding fragments thereof, which recognize a fraction of TCR Vα chains comprising at least two different two different TCR Vα chains but less than all TCR Vα chains or which recognize a fraction of TCR Vβ chains comprising at least two different two different TCR Vβ chains but less than all TCR Vβ chains.

The invention therefore also contemplates using TCR Vα chain antibodies and binding fragments thereof or TCR Vβ chain antibodies and binding fragments thereof binding substantially to the same epitope or parts of the same epitope as do the TCR Vα binding antibodies and binding fragments or TCR Vβ chain antibodies and binding fragments thereof as described above. Thus the invention relates TCR Vβ chain antibodies and binding fragments thereof binding substantially to the same epitope or parts of the same epitope as 15B4 or 5H4. The invention relates to antibodies and binding fragments thereof binding substantially to the same epitope or parts of the same epitope as 15B4 or 5H4.

Further, the invention considers using TCR Vα chain antibodies and binding fragments thereof or TCR Vβ chain antibodies and binding fragments thereof competing with TCR Vα chain antibodies and binding fragments thereof or TCR Vβ chain antibodies and binding fragments thereof as described above. Thus the invention relates to TCR Vα chain antibodies and binding fragments thereof or TCR Vβ chain antibodies and binding fragments thereof antibodies and binding fragments thereof competing with 15B4 or 5H4.

Epitope mapping may be undertaken by producing different fragments of the antigen such as the TCR Vα chain or the TCR Vβ chain and to then test these fragments for binding to antibodies or the binding fragments thereof. Binding may be measured using a Biacore® interaction analysis. One may also use commercially available peptide arrays such as PepSpot™ from JPT Peptide Technologies GmbH (Berlin, Germany), or proteomics-based mass spectrometry methods. Competition for binding to a particular antigen or epitope can be determined using assays known in the art. For example one may label an antibody in accordance with the invention and test for its binding to TCR Vα chain or TCR Vβ chain. Subsequently, one adds unlabeled 15B4 (or any other TCR Vα chain or TCR Vβ chain antibody) and determines whether it affects binding of the labeled antibody, or binding of the labeled antibody is studied in presence or absence of various concentrations of such unlabeled TCR Vα chain or TCR Vβ chain binding antibody. Such label could be radioactive or fluorescent or other kinds of detectable label.

Competition for binding to a particular antigen or epitope is determined by a reduction in binding to antigen or epitope of at least about 50%, or at least about 70%, or at least about 80%, or at least about 90%, or at least about 95%, or at least about 99% or about 100% for the antibody in accordance with the invention. Binding may be measured using Biacore® equipment, various fluorescence detection technologies (e.g. fluorescence correlation spectroscopy, fluorescence cross-correlation, fluorescence lifetime measurements etc.) or various types of radioimmunoassays or other assays used to follow antibody binding to a target molecule.

As mentioned above, the present invention considers cluster-specific TCR Vα chain or TCR Vβ chain antibodies or binding fragments thereof. A full-length antibody includes a constant domain and a variable domain. The constant region need not be present in an antigen binding fragment of an antibody.

Binding fragments may thus include portions of an intact full length antibody, such as an antigen binding or variable region of the complete antibody. Examples of antibody fragments include Fab, F(ab′)₂, Id and Fv fragments; diabodies; linear antibodies; single-chain antibody molecules (e.g., scFv); multispecific antibody fragments such as bispecific, trispecific, and multispecific antibodies (e.g., diabodies, triabodies, tetrabodies); minibodies; chelating recombinant antibodies; tribodies or bibodies; intrabodies; nanobodies; small modular immunopharmaceuticals (SMIP), binding-domain immunoglobulin fusion proteins; camelized antibodies; VHH containing antibodies; chimeric antigen receptor (CAR); and any other polypeptides formed from antibody fragments. The skilled person is aware that the antigen-binding function of an antibody can be performed by fragments of a full-length antibody.

A Fab fragment consists of the VL, VH, CL and CH1 domains. An F(ab′)² fragment comprises two Fab fragments linked by a disulfide bridge at the hinge region. An Fd is the VH and CH1 domains of a single arm of an antibody. An Fv fragment is the VL and VH domains of a single arm of an antibody.

Binding fragments also encompass monovalent or multivalent, or monomeric or multimeric (e.g. tetrameric), CDR-derived binding domains.

A bispecific antibody comprises two different binding specificities and thus binds to two different antigens. In one embodiment, the bispecific antibody comprises a first antigen recognition domain that binds to a first antigen and a second antigen recognition domain that binds to a second antigen. In one embodiment, the first antigen recognition domain binds to a fraction of T cell TCR Vα chains as defined herein and the second antigen recognition region binds to a fraction of T cell TCR Vα chains as defined herein which comprises at least one different TCR Vα chain as the fraction of T cell TCR Vα chains that is recognized by the first antigen recognition domain. In one embodiment, the first antigen recognition domain binds to a fraction of T cell TCR Vβ chains as defined herein and the second antigen recognition region binds to a fraction of T cell TCR Vβ chains as defined herein which comprises at least one different TCR Vβ chain as the fraction of T cell TCR Vβ chains that is recognized by the first antigen recognition domain.

In some instances, a bispecific antibody that recognizes a T cell antigen is referred to as a Bispecific T Cell Engager (BiTE). The present invention is not limited by the use of any particular bispecific antibody. Rather, any bispecific antibody or BiTE can be used. One of the scFvs binds to T cells via the CD3 receptor, and the other to the antigen to be targeted via an antigen specific molecule. This causes T cells to exert cytotoxic activity on cells expressing the targeted antigen by producing proteins like perforin and granzymes, independently of the presence of MHC I or co-stimulatory molecules. Examples of TCR Vα chains or TCR Vβ chains are described elsewhere herein, all of which may be targeted by the bispecific antibody. In one embodiment, the bispecific antibody comprises a human antibody, a humanized antibody, or fragments thereof.

In one embodiment, the first antigen recognition domain binds to a fraction of T cell TCR Vβ chains and the second antigen recognition region binds to an antigen recognition region binds to CD3 on T cells. Methods for making bispecific antibodies are known to the skilled person in the art. Bispecific antibodies can be produced recombinantly using the co-expression of two immunoglobulin heavy chain/light chain pairs, as for example described in Milstein et al. (1983; Nature 305:537). Alternatively, bispecific antibodies can be prepared using chemical linkage (see, e.g., Brennan et al. (1985)). Bispecific antibodies include bispecific antibody fragments (see, e.g., Holliger et al. (1993) Proc. Natl. Acad. Sci. U.S.A. 90:6444-48, Gruber et al. (1994) J. Immunol. 152:5368.)

A chimeric antigen receptor CAR comprises an antigen binding domain derived from a bispecific antibody, a transmembrane domain, and a CD3 zeta signaling domain. More specifically the term “chimeric antigen receptors (CARs),” as used herein, refers for example to chimeric T-cell receptors, artificial T-cell receptors, or chimeric immunoreceptors. CARs may be used for mediating the specificity of a monoclonal antibody onto a T cell. In specific embodiments of the invention, CARs direct specificity of the cell to TCR Vα chains or TCR Vβ chains, for example. In some embodiments, CARs comprise an intracellular activation domain, a transmembrane domain, and an extracellular domain comprising a binding region directed to TCR Vα chains or TCR Vβ chains. In particular aspects, CARs comprise fusions of single-chain variable fragments (scFv) derived from monoclonal antibodies, fused to CD3-zeta a transmembrane domain and endodomain. In certain cases, the spacing of the antigen-recognition domain can be modified to reduce activation-induced cell death. In certain cases, CARs comprise domains for additional co-stimulatory signaling, such as CD3-zeta, FcR, CD27, CD28, CD137, DAP10, and/or OX40. It is contemplated by the invention that a CAR could be used for enhancing the effect of the antibody or fragment of the invention. For example, if an antibody that binds to a fraction of T cell receptor variable alpha (TCR Vα) chains comprising at least two different TCR Vα chains but less than all TCR Vα chains or that binds to a fraction of T cell receptor variable beta (TCR Vβ) chains comprising at least two different TCR Vβ chains but less than all TCR Vβ chains shows no or only little activity of T cell depletion, its binding domain can be integrated into a CAR in order to elicit or enhance its T cell depletion capability. It is also envisioned that the activity of an antibody of the invention that is considerably effective, for example in depleting specific T cells, is further enhanced by the integration of its binding domain or fragments and/or variations thereof into a CAR.

The TCR variable chain binding antibodies and binding fragments thereof may also encompass variants of the exemplary antibodies, binding fragments and sequences disclosed herein. Variants include peptides and polypeptides comprising one or more amino acid sequence substitutions, deletions, and/or additions that have the same or substantially the same affinity and specificity of epitope binding as one or more of the exemplary antibodies, fragments and sequences disclosed herein. Thus, variants include peptides and polypeptides comprising one or more amino acid sequence substitutions, deletions, and/or additions to the exemplary antibodies, fragments and sequences disclosed herein where such substitutions, deletions and/or additions do not cause substantial changes in affinity and specificity of epitope binding. For example, a variant of an antibody or fragment may result from one or more changes to an antibody or fragment comprising one or more of amino acid sequence of SEQ ID NOs: 218, 219 or 232, 233 or where the changed antibody or fragment has the same or substantially the same affinity and specificity of epitope binding as the starting sequence.

Antibodies or binding fragments thereof as far as they are generally referred to in the context of the present invention may also be part of larger immunoadhesion molecules, formed by covalent or non-covalent association of the antibody or antibody portion with e.g. one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibodies and fragments comprising immunoadhesion molecules can be obtained using standard recombinant DNA techniques, as described herein. Preferred antigen binding portions are complete domains or pairs of complete domains.

The binding antibodies and binding fragments of the present invention may also encompass domain antibody (dAb) fragments (Ward et al., Nature 341:544-546, 1989) which consist of a V_{H} domain. The antibodies and binding fragments of the present invention also encompass diabodies are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., EP 404,097; WO 93/11161; Holliger et al., Proc. Natl. Acad. Sci. USA 90:6444-6448, 1993, and Poljak et al., Structure 2:1121-1123, 1994). Diabodies can be bispecific or monospecific.

As mentioned the antibodies and binding fragments of the present invention also encompass single-chain antibody fragments (scFv). An scFv comprises an antibody heavy chain variable region (V_{H}) operably linked to an antibody light chain variable region (V_{L}) wherein the heavy chain variable region and the light chain variable region, together or individually, form a binding site. A scFv may comprise a V_{H} region at the amino-terminal end and a V_{L} region at the carboxy-terminal end. Alternatively, scFv may comprise a V_{L} region at the amino-terminal end and a V_{H} region at the carboxy-terminal end. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883).

A scFv may optionally further comprise a polypeptide linker between the heavy chain variable region and the light chain variable region. Such polypeptide linkers generally comprise between 1 and 50 amino acids, alternatively between 3 and 12 amino acids, alternatively 2 amino acids. An example of a linker peptide for linking heavy and light chains in a scFv comprises the 5 amino acid sequence Gly-Gly-Gly-Gly-Ser (SEQ ID NO: 238). Other examples comprise one or more tandem repeats of this sequence (for example, a polypeptide comprising two to four repeats of Gly-Gly-Gly-Gly-Ser (SEQ ID NO: 238) to create linkers.

The antibodies and binding fragments of the present invention also encompass heavy chain antibodies (HCAb). Exceptions to the H₂L₂ structure of conventional antibodies occur in some isotypes of the immunoglobulins found in camelids (camels, dromedaries and llamas; Hamers-Casterman et al., 1993 Nature 363: 446; Nguyen et al., 1998 J. Mol. Biol. 275: 413), wobbegong sharks (Nuttall et al., Mol Immunol. 38:313-26, 2001), nurse sharks (Greenberg et al., Nature 374:168-73, 1995; Roux et al., 1998 Proc. Nat. Acad. Sci. USA 95: 11804), and in the spotted ratfish (Nguyen, et al., “Heavy-chain antibodies in Camelidae; a case of evolutionary innovation,” 2002 Immunogenetics 54(1): 39-47). These antibodies can apparently form antigen-binding regions using only heavy chain variable region, in that these functional antibodies are dimers of heavy chains only (referred to as “heavy-chain antibodies” or “HCAbs”). Accordingly, some embodiments of the present antibodies and binding fragments may be heavy chain antibodies (HCAb) that specifically bind to the TCR. For example, heavy chain antibodies that are a class of IgG and devoid of light chains are produced by animals of the genus Camelidae which includes camels, dromedaries and llamas (Hamers-Casterman et al., Nature 363:446-448 (1993)). HCAbs have a molecular weight of about 95 kDa instead of the about 160 kDa molecular weight of conventional IgG antibodies. Their binding domains consist only of the heavy-chain variable domains, often referred to as V_{HH} to distinguish them from conventional V_{H}, Muyldermans et al., J. Mol. Recognit. 12:131-140 (1999). The variable domain of the heavy-chain antibodies is sometimes referred to as a nanobody (Cortez-Retamozo et al., Cancer Research 64:2853-57, 2004). A nanobody library may be generated from an immunized dromedary as described in Conrath et al., (Antimicrob Agents Chemother 45: 2807-12, 2001) or using recombinant methods.

Since the first constant domain (C_{H1}) is absent (spliced out during mRNA processing due to loss of a splice consensus signal), the variable domain (V_{HH}) is immediately followed by the hinge region, the C_{H2} and the C_{H3} domains (Nguyen et al., Mol. Immunol. 36:515-524 (1999); Woolven et al., Immunogenetics 50:98-101 (1999)). Camelid V_{HH} reportedly recombines with IgG2 and IgG3 constant regions that contain hinge, CH2, and CH3 domains and lack a CH1 domain (Hamers-Casterman et al., supra). For example, llama IgG1 is a conventional (H₂L₂) antibody isotype in which V_{H} recombines with a constant region that contains hinge, CH1, CH2 and CH3 domains, whereas the llama IgG2 and IgG3 are heavy chain-only isotypes that lack CH1 domains and that contain no light chains.

Although the HCAbs are devoid of light chains, they have an antigen-binding repertoire. The genetic generation mechanism of HCAbs is reviewed in Nguyen et al. Adv. Immunol 79:261-296 (2001) and Nguyen et al., Immunogenetics 54:39-47 (2002). Sharks, including the nurse shark, display similar antigen receptor-containing single monomeric V-domains. Irving et al., J. Immunol. Methods 248:31-45 (2001); Roux et al., Proc. Natl. Acad. Sci. USA 95:11804 (1998).

V_{HH}s comprise small intact antigen-binding fragments (for example, fragments that are about 15 kDa, 118-136 residues). Camelid V_{HH} domains have been found to bind to antigen with high affinity (Desmyter et al., J Biol. Chem. 276:26285-90, 2001), with V_{HH} affinities typically in the nanomolar range and comparable with those of Fab and scFv fragments. V_{HH}s are highly soluble and more stable than the corresponding derivatives of scFv and Fab fragments. V_{H} fragments have been relatively difficult to produce in soluble form, but improvements in solubility and specific binding can be obtained when framework residues are altered to be more V_{HH}-like (see, for example, Reichman et al., J Immunol Methods 1999, 231:25-38). V_{HH}s carry amino acid substitutions that make them more hydrophilic and prevent prolonged interaction with BiP (Immunoglobulin heavy-chain binding protein), which normally binds to the H-chain in the Endoplasmic Reticulum (ER) during folding and assembly, until it is displaced by the L-chain. Because of the VHHs' increased hydrophilicity, secretion from the ER is improved.

Functional VHHs may be obtained by proteolytic cleavage of HCAb of an immunized camelid, by direct cloning of V_{HH} genes from B-cells of an immunized camelid resulting in recombinant V_{HH}s, or from naive or synthetic libraries. VHHs with desired antigen specificity may also be obtained through phage display methodology. Using V_{HH}s in phage display is much simpler and more efficient compared to Fabs or scFvs, since only one domain needs to be cloned and expressed to obtain a functional antigen-binding fragment. Muyldermans, Biotechnol. 74:277-302 (2001); Ghahroudi et al., FEBS Lett. 414:521-526 (1997); and van der Linden et al., J. Biotechnol. 80:261-270 (2000). Methods for generating antibodies having camelid heavy chains are also described in U.S. Patent Publication Nos. 20050136049 and 20050037421.

The binding antibodies and binding fragments thereof may also encompass any of the e.g. foregoing specifically mentioned amino acid sequences of the light or heavy chains with one or more conservative substitutions (e.g., 1, 2, 3, 4, 5, 6, 7, 8, 9, 10, 11, 12, 13, 14, or 15 conservative substitutions). One can determine the positions of an amino acid sequence that are candidates for conservative substitutions, and one can select synthetic and naturally-occurring amino acids that effect conservative substitutions for any particular amino acids. Consideration for selecting conservative substitutions include the context in which any particular amino acid substitution is made, the hydrophobicity or polarity of the side-chain, the general size of the side chain, and the pK value of side-chains with acidic or basic character under physiological conditions. For example, lysine, arginine, and histidine are often suitably substituted for each other. As is known in the art, this is because all three amino acids have basic side chains, whereas the pK value for the side-chains of lysine and arginine are much closer to each other (about 10 and 12) than to histidine (about 6). Similarly, glycine, alanine, valine, leucine, and isoleucine are often suitably substituted for each other, with the proviso that glycine is frequently not suitably substituted for the other members of the group. Other groups of amino acids frequently suitably substituted for each other include, but are not limited to, the group consisting of glutamic and aspartic acids; the group consisting of phenylalanine, tyrosine, and tryptophan; and the group consisting of serine, threonine, and, optionally, tyrosine.

By making conservative modifications to the amino acid sequence or corresponding modifications to the encoding nucleotides, one can produce antibodies or binding fragments thereof having functional and chemical characteristics similar to those of the exemplary antibodies and fragments disclosed herein.

The binding antibodies and binding fragments thereof as they are mentioned in the context of the present invention may encompass derivatives of the exemplary antibodies, fragments and sequences disclosed herein. Derivatives include polypeptides or peptides, or variants, fragments or derivatives thereof, which have been chemically modified. Examples include covalent attachment of one or more polymers, such as water soluble polymers, N-linked, or O-linked carbohydrates, sugars, phosphates, and/or other such molecules such as detectable labels such as fluorophores.

Labeling agents may be coupled either directly or indirectly to the antibodies or antigens of the invention. One example of indirect coupling is by use of a spacer moiety. Furthermore, the antibodies of the present invention can comprise a further domain, said domain being linked by covalent or noncovalent bonds. The linkage can be based on genetic fusion according to the methods known in the art and described above or can be performed by, e.g., chemical cross-linking as described in, e.g., international application WO 94/04686. The additional domain present in the fusion protein comprising the antibody of the invention may preferably be linked by a flexible linker, advantageously a polypeptide linker, wherein said polypeptide linker comprises plural, hydrophilic, peptide-bonded amino acids of a length sufficient to span the distance between the C-terminal end of said further domain and the N-terminal end of the antibody of the invention or vice versa. The therapeutically or diagnostically active agent can be coupled to the antibody of the invention or an antigen-binding fragment thereof by various means. This includes, for example, single-chain fusion proteins comprising the variable regions of the antibody of the invention coupled by covalent methods, such as peptide linkages, to the therapeutically or diagnostically active agent. Further examples include molecules which comprise at least an antigen-binding fragment coupled to additional molecules covalently or non-covalently include those in the following non-limiting illustrative list. Traunecker et al., Int. J. Cancer Surp. SurP 7 (1992), 51-52, describe the bispecific reagent janusin in which the Fv region directed to CD3 is coupled to soluble CD4 or to other ligands such as OVCA and IL-7. Similarly an Fv region directed to TCR Vα chains or TCR Vβ chains may be coupled to portions of e.g. an anti-CD40 agonistic antibody and/or portions of an anti-CTLA4 antagonistic antibody. Similarly, the variable regions of the antibody of the invention can be constructed into Fv molecules and coupled to alternative ligands such as those illustrated in the cited article. Higgins et al., J. Infect Disease 166 (1992), 198-202, described a hetero-conjugated antibody composed of OKT3 cross-linked to an antibody directed to a specific sequence in the V3 region of GP120. Such hetero-conjugate antibodies can also be constructed using at least the variable regions contained in the antibody of the invention methods. Additional examples of specific antibodies include those described by Fanger et al., Cancer Treat. Res. 68 (1993), 181-194 and by Fanger et al., Crit. Rev. Immunol. 12 (1992), 101-124. Conjugates that are immunotoxins including conventional antibodies have been widely described in the art. The toxins may be coupled to the antibodies by conventional coupling techniques or immunotoxins containing protein toxin portions can be produced as fusion proteins. The antibodies of the present invention can be used in a corresponding way to obtain such immunotoxins. Illustrative of such immunotoxins are those described by Byers et al., Seminars Cell. Biol. 2 (1991), 59-70 and by Fanger et al., Immunol. Today 12 (1991), 51-54.

The above described fusion proteins may further comprise a cleavable linker or cleavage site for proteases. These spacer moieties, in turn, can be either insoluble or soluble (Diener et al., Science 231 (1986), 148) and can be selected to enable drug release from the antigen at the target site.

Examples of therapeutic agents which can be coupled to the antibodies and antigens of the present invention for immunotherapy are drugs, radioisotopes, lectins, and toxins. The drugs with which can be conjugated to the antibodies and antigens of the present invention include compounds which are classically referred to as drugs such as mitomycin C, daunorubicin, and vinblastine. In using radioisotopically conjugated antibodies or antigens of the invention for, e.g., tumor immunotherapy, certain isotopes may be more preferable than others depending on such factors as leukocyte distribution as well as stability and emission.

Some emitters may be preferable to others. In general, alpha and beta particle emitting radioisotopes are preferred in immunotherapy. Preferred are short range high energy a emitters such as ²¹²Bi. Examples of radioisotopes which can be bound to the antibodies or antigens of the invention for therapeutic purposes are ¹²⁵I, ¹³¹I, ⁹⁰Y, ⁶⁷Cu, ²¹²Bi, ²¹²At, ²¹¹Pb, ⁴⁷Sc, ¹⁰⁹Pd and ¹⁸⁸Re. Other therapeutic agents which can be coupled to the antibody or antigen of the invention, as well as ex vivo and in vivo therapeutic protocols, are known, or can be easily ascertained, by those of ordinary skill in the art.

As mentioned, the invention also relates in some embodiment to nucleic acid molecules encoding antibodies and binding fragments thereof, vectors comprising such nucleic acid molecules and host cells comprising such nucleic acid sequences and vectors.

The antibodies and binding fragments thereof may be encoded by a single nucleic acid (e.g., a single nucleic acid comprising nucleotide sequences that encode the light and heavy chain polypeptides of the antibody), or by two or more separate nucleic acids, each of which encode a different part of the antibody or antibody fragment. In this regard, the invention provides one or more nucleic acids that encode any of the forgoing antibodies, or binding fragments. The nucleic acid molecules may be DNA, cDNA, RNA and the like.

According to one aspect of the invention, the invention provides a nucleic acid that encodes a heavy chain region of an antibody or a portion thereof. Exemplary nucleic acid sequences are provided in SEQ ID Nos: 223 and 237. The invention also provides a nucleic acid that encodes a light chain variable region of an antibody or a portion thereof. Exemplary nucleic acid sequences are provided in SEQ ID Nos.:222 and 236.

Also encompassed by the invention are nucleic acids encoding any of the foregoing amino acid sequences of the light or heavy chains that comprise one or more conservative substitutions (e.g., 1, 2, 3, 4, 5, 6, 7, 8, 9, 10, 11, 12, 13, 14, or 15 conservative substitutions), as discussed with respect to the antibody and antibody fragment of the invention, where the antibody or fragment comprising the substitution has the same or substantially the same affinity and specificity of epitope binding as one or more of the exemplary antibodies, fragments and sequences disclosed herein.

Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally associated or heterologous promoter regions.

The nucleic acids described herein can be inserted into vectors, e.g., nucleic acid expression vectors and/or targeting vectors. Such vectors can be used in various ways, e.g., for the expression of an antibody or a binding fragment in a cell or transgenic animal. Accordingly, the invention provides a vector comprising any one or more of the nucleic acids of the invention. A “vector” is any molecule or composition that has the ability to carry a nucleic acid sequence into a suitable host cell where synthesis of the encoded polypeptide can take place. Typically and preferably, a vector is a nucleic acid that has been engineered, using recombinant DNA techniques that are known in the art, to incorporate a desired nucleic acid sequence (e.g., a nucleic acid of the invention). Desirably, the vector is comprised of DNA. However, vectors that are not based on nucleic acids, such as liposomes, are also known in the art and can be used in connection with the invention. The inventive vector can be based on a single type of nucleic acid (e.g., a plasmid) or non-nucleic acid molecule (e.g., a lipid or a polymer). Alternatively, the vector can be a combination of a nucleic acid and a non-nucleic acid (i.e., a “chimeric” vector). For example, a plasmid harboring the nucleic acid can be formulated with a lipid or a polymer as a delivery vehicle. Such a vector is referred to herein as a “plasmid-lipid complex” and a “plasmid-polymer” complex, respectively. The inventive gene transfer vector can be integrated into the host cell genome or can be present in the host cell in the form of an episome.

Vectors are typically selected to be functional in the host cell in which the vector will be used (the vector is compatible with the host cell machinery such that amplification of the gene and/or expression of the gene can occur). A nucleic acid molecule encoding an antibody or binding fragment thereof may be amplified/expressed in prokaryotic, yeast, insect (baculovirus systems) and/or eukaryotic host cells. Selection of the host cell will depend in part on whether the antibody or fragment is to be post-transitionally modified (e.g., glycosylated and/or phosphorylated). If so, yeast, insect, or mammalian host cells are preferable.

Expression vectors typically contain one or more of the following components (if they are not already provided by the nucleic acid molecules): a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

The invention in some aspects further provides a cell (e.g., an isolated or purified cell) comprising a nucleic acid or vector of the invention. The cell can be any type of cell capable of being transformed with the nucleic acid or vector of the invention so as to produce a polypeptide encoded thereby. The cell is preferably the cell of a mammal, such as a human, and is more preferably a hybridoma cell, an embryonic stem cell, or a fertilized egg. The embryonic stem cell or fertilized egg may not be a human embryonic stem cell or a human fertilized egg.

The host cells may be prokaryotic host cells (such as E. coli) or eukaryotic host cells (such as a yeast cell, an insect cell, or a vertebrate cell). The host cell, when cultured under appropriate conditions, expresses an antibody or binding fragment which can subsequently be collected from the culture medium (if the host cell secretes it into the medium) or directly from the host cell producing it (if it is not secreted). Selection of an appropriate host cell will depend upon various factors, such as desired expression levels, polypeptide modifications that are desirable or necessary for activity, such as glycosylation or phosphorylation, and ease of folding into a biologically active molecule. A number of suitable host cells are known in the art and many are available from the American Type Culture Collection (ATCC), Manassas, Va. Examples include mammalian cells, such as Chinese hamster ovary cells (CHO) (ATCC No. CCL61) CHO DHFR-cells (Urlaub et al. Proc. Natl. Acad. Sci. USA 97, 4216-4220 (1980)), human embryonic kidney (HEK) 293 or 293T cells (ATCC No. CRL1573), 3T3 cells (ATCC No. CCL92), or PER.C6 cells.

The cell comprising the nucleic acid or vector of the invention can be used to produce the antibody or binding fragment thereof, or a portion thereof (e.g., a heavy chain sequence, or a light chain sequence encoded by the nucleic acid or vector). After introducing the nucleic acid or vector of the invention into the cell, the cell is cultured under conditions suitable for expression of the encoded sequence. The antibody, antigen binding fragment, or portion of the antibody then can be isolated from the cell.

Another aspect of the invention relates to the use of an antibody or binding fragment thereof according to any one of the preceding claims for depleting a subpopulation of T cells expressing a fraction of TCR Vα chains comprising at least two different TCR Vα chains or for depleting a subpopulation of T cells expressing a fraction of TCR Vβ chains comprising at least two different TCR Vβ chains.

Another aspect of the invention relates to the use of an antibody or binding fragment thereof according to any one of the preceding claims for ex vivo depleting a subpopulation of T cells expressing a fraction of TCR Vα chains comprising at least two different TCR Vα chains or for depleting a subpopulation of T cells expressing a fraction of TCR Vβ chains comprising at least two different TCR Vβ chains.

A further aspect of the invention relates to an antibody or binding fragment thereof as described herein for use as a medicament.

A specific embodiment relates to an antibody or binding fragment thereof according to any one of the preceding claims for use in the treatment of T cell leukemia.

The binding data showing that CL 1 binds to Jurkat cells, which are T cell leukemia cells and hence are an established model for T-cell leukemia, show that the antibody or binding fragments according to the invention are targeting T cell leukemia cells. Therefore, the antibodies or binding fragments of the invention can be used for the treatment of T cell mediated diseases such as T cell leukemia.

In vivo depletion experiments in mouse are suitable to prove that it is feasible to deplete specific T cell populations, such as aberrant T cells causing T cell leukemia, in vivo.

Further the ADCC assay monitors the capability of the antibody of the invention to trigger the ADCC, i.e. the active lysis of a target cells, e.g. malignant T cells.

The TCR variable chain binding antibodies or binding fragments thereof can be formulated in compositions, especially pharmaceutical compositions. Such compositions comprise a therapeutically or prophylactically effective amount of an antibody or binding fragment thereof in admixture with a suitable carrier, e.g., a pharmaceutically acceptable agent.

Pharmaceutically acceptable agents for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in liquid form or in a lyophilized or freeze-dried form and may include one or more lyoprotectants, excipients, surfactants, high molecular weight structural additives and/or bulking agents (see for example U.S. Pat. Nos. 6,685,940, 6,566,329, and 6,372,716).

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, antioxidants, chelating agents, inert gases and the like. See generally, Remington's Pharmaceutical Science, 16th Ed., Mack Eds., 1980, which is incorporated herein by reference.

Pharmaceutical compositions described herein can be formulated for controlled or sustained delivery in a manner that provides local concentration of the product (e.g., bolus, depot effect) and/or increased stability or half-life in a particular local environment. The compositions can include the formulation of antibodies, binding fragments, nucleic acids, or vectors of the invention with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection.

Both biodegradable and non-biodegradable polymeric matrices can be used to deliver compositions of the present invention, and such polymeric matrices may comprise natural or synthetic polymers. Biodegradable matrices are preferred. The period of time over which release occurs is based on selection of the polymer. Typically, release over a period ranging from between a few hours and three to twelve months is most desirable.

Alternatively or additionally, the compositions can be administered locally via implantation into the affected area of a membrane, sponge, or other appropriate material on to which an antibody, binding fragment, nucleic acid, or vector of the invention has been absorbed or encapsulated. Where an implantation device is used, the device can be implanted into any suitable tissue or organ, and delivery of an antibody, binding fragment, nucleic acid, or vector of the invention can be directly through the device via bolus, or via continuous administration, or via catheter using continuous infusion.

A pharmaceutical composition comprising a binding antibody or binding fragment thereof can be formulated for inhalation, such as for example, as a dry powder. Inhalation solutions also can be formulated in a liquefied propellant for aerosol delivery. In yet another formulation, solutions may be nebulized.

Certain formulations containing antibodies or binding fragments thereof can be administered orally. Formulations administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of a selective binding agent. Diluents, flavorings, low melting point waxes, vegetable oils, lubricants, suspending agents, tablet disintegrating agents, and binders also can be employed.

Antibody or binding fragment thereof that binds to a fraction of TCR Vβ chains comprising at least two different TCR Vβ chains but less than all TCR Vβ chains has an EC₅₀ of about 0.08 nm to about 1000 nM, preferably of about 0.1 to about 900 nM, more preferably of about 0.4 to about 800 nM, such as about 0.4 to about 400 nM, about 0.08 nM to about 0.8 nM, or about 0.1 nM to about 0.6 nM. The EC50 values of the rat antibodies may vary from the chimeric or humanized antibody version. The EC50 value of the chimeric or humanized antibodies according to the invention may be of about 0.4 to about 800 nM, preferably 100 to about 500 nM, more preferably of about 150 nM to 350 nM.

The invention is now described with respect to some examples which are however not be construed as limiting.

ADCC refers to antibody-dependent cellular cytotoxicity. In order to determine whether an antibody is in principle capable of mediating ADCC, ADCC may be measured in vitro by a luciferase assay monitoring the activation of gene transcription through the NFAT (nuclear factor of activated T-cells) pathway in the effector cell. For example, the ADCC Reporter Bioassay (Promega) uses engineered Jurkat cells stably expressing the FcγRIIIa receptor, V158 (high affinity) variant, and an NFAT response element driving expression of firefly luciferasease effector cells. The biological activity of the antibody in ADCC MOA is quantified through the luciferase produced as a result of NFAT pathway activation;

In addition ADCC could be measured by so-called Cr⁵¹, Eu, S³⁵, and Calcein-release assays. A target cell displaying the antigen of interest on its surface may be labeled with these compounds. After binding of the therapeutic antibody, the cells are washed and effector cells expressing Fc receptors such as FcγRIII are co incubated with the antibody-labeled target cells and lysis of the target cells can be monitored by release of the labels. Another approach uses the so-called aCella TOX™ assay.

CDC refers to complement-dependent cellular cytotoxicity. In order to determine whether an antibody is in principle capable of mediating CDC, CDC may be measured in vitro as described e.g. in Delobel A et al, Methods Mol Biol. (2013); 988:115-43 or Current Protocols in Immunology, Chapter 13 Complement

### (Print ISSN: 1934-3671).

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

The above-mentioned CDRs of a light and heavy chain variable region are preferably embedded in the framework and constant region of a human-derived antibody, i.e. in the sequences as determined for antibodies obtained from human patients as described herein. Preferably these antibodies are of the IgG class.

However, the above-mentioned CDRs of a light and heavy chain variable region may also be embedded in human sequences of framework and constant regions derived from other human antibodies, particularly if such sequences have been shown to be effective in antibody dependent cell mediated cytotoxicity (ADCC). In this context, one may e.g. use the human constant and framework sequences of humanized therapeutic antibodies that have been successfully used for therapeutic applications. The above-mentioned CDRs of a light and heavy chain variable region are preferably incorporated into the framework and constant regions of such humanized antibodies of the human IgG class.

Further, the above-mentioned CDRs of a light and heavy chain variable region may be embedded in essentially human sequences for framework and constant regions. However, particularly the framework regions, but also the constant regions may comprise amino acids as they are e.g. typically found in mouse antibodies which are known to enhance antigen binding and/or e.g. ADCC (see e.g. European patent application EP 0 451 216). Preferably these antibodies are of the IgG class.

The antibody may trigger antibody dependent cytotoxicity (ADCC) and/or CDC complement dependent cytotoxicity and/or antibody dependent cellular phagocytosis (ADCP) phagocytosis. In a specific embodiment of the present application the antibody triggers ADCC.

### Method for Generating an Antibody

A further aspect of the invention refers to a method for generating an antibody binding to a cell surface protein of interest, the method comprising the following steps:
(a) providing a non-human cell which does not express the endogenous form of the cell surface protein of interest but expresses an exogenous form of the cell surface protein of interest comprising at least one human segment;
(b) immunization of a non-human animal with the cell line provided in step (a);
(c) generation of hybridomas from the immunized non-human animal of step (b);
(d) screening for an antibody that binds to the surface protein of interest by contacting the antibodies secreted by the hybridomas of step (c) with human cells which do not express the endogenous form of the cell surface protein of interest but express an exogenous form of the cell surface protein of interest comprising at least one human segment.

Providing a non-human cell which does not express the endogenous form of the cell surface protein of interest means that the non-human cell is substantially incapable of producing the endogenous form of the protein but is capable of producing an exogenous form of the cell surface protein of interest. The skilled person is aware of different methods to inhibit the expression of the endogenous form of the protein. Also isolated cell lines, not expressing the endogenous form of the surface protein of interest that arose spontaneously can be used. Typically, in the non-human cell line the gene locus/loci of the surface protein of interest has/have been disabled.

The term “at least one human segment” as used herein refers to at least one part or region of the protein. This means that both completely human cell surface proteins and cell surface proteins that are not completely human are envisaged by the invention. Accordingly, the cell surface protein may comprise in addition to the at least one human segment, segments of another origin. For example, the intracellular domain and transmembrane domain of cell surface protein may be of mouse origin and the extracellular domain may be of human origin. For example, the constant regions of a TCR may be of mouse origin, while the variable domains may be of human origin. The term “segment” as used herein refers to parts of a protein such as, without limitation, domains or sequence stretches.

The term “cell surface protein of interest” refers to any protein that is known by the skilled person as a cell surface protein. “Cell surface protein” as used herein is a protein of which at least one part is exposed to the extracellular environment. The protein may be embedded in the lipid layer of the cell membrane or may bind to a molecule which is integrated in the lipid layer. The cell surface protein of interest may be a dimer, preferably a heterodimer, such as the TCR. An exemplary embodiment of the cell surface protein is the TCR. In a specific embodiment the cell surface protein is not CD5.

The exogenous form of the cell surface protein of interest can be expressed transiently or permanently. The skilled person is familiar with techniques of permanent or transient expression of genes.

The preparation of the monoclonal antibodies may be carried out based on known methods (C. Milstein, G. Kohler, Nature 256 (1975) 495). As immunogen a non-human cell which does not express the endogenous form of the cell surface protein of interest but expresses an exogenous form of the cell surface protein of interest comprising at least one human segment is used.

The term “non-human cell line” as used herein refers to any non-human cell line that is known to the skilled in the art which is suitable for immunization of a non-human animal. For example mouse or rat cell lines may be used.

Examples for non-human animals that may be immunized are cattle, sheep, goat, lama, pig, horse, mouse, rat, fowl, monkey, rabbit and the like. In a preferred embodiment, rat, mouse, rabbit or lama may be immunized. In a more preferred embodiment a rat may be immunized. In the rat a high number of spleen cells, in particular a higher number of spleen cells compared to a mouse, can be obtained.

In one embodiment, the non-human animal that is immunized in step (b) is of another species than the non-human cell line provided in step (a). For example the immunization of rats with a mouse cell line has the advantage that a strong immune response is triggered in the rat by the mouse cell line.

In a particular embodiment, the non-human animal to be immunized is a rat and the non-human cell line used for immunization is a mouse cell line. Also other combinations of non-human animals to be immunized and non-human cell lines can be used.

Screening for an antibody that binds to the surface protein of interest may be carried out by the use flow cytometry in particular, by FACS. The antibody is secreted by the hybridomas of step (c) is thereby contacted with a human cell line. The non-human cell line used for immunization is not used for screening, since this cell line also binds antibodies which are not specific for the cell surface protein of interest. A human cell line expressing the cell surface protein of interest which is used in the screening step is advantageous since antibodies specific for the human cell surface protein of interest bind to this cell line, but antibodies not specific for the human cell surface protein of interest bind substantially not to this human cell line. Hence, using the human cell line in the screening step allows differentiating between antibodies which bind specifically to the cell surface protein of interest and antibodies which bind non-specifically.

In order to make the screening step more efficient, supernatants of several plates can be pooled and be analyzed in a single step. For example the supernatant of 2, of 3, of 4, of 5, of 6, of 7, of 8, of 9, of 10 or more wells can be pooled. Preferably the supernatants of 4 wells can be pooled and analyzed in a primary screening step. If a supernatant pooled from several wells shows binding of an antibody, the supernatants of the single wells may be analyzed individually in a secondary screening step.

The antibodies secreted by the hybridomas of step (c) may be contacted with a mixture of human cells which do not express the endogenous form of the cell surface protein of interest comprising:
(i) a first defined proportion of the mixture of human cells which expresses the functional cell surface protein of interest; and
(ii) a second defined proportion of the mixture of human cells which does not express a functional cell surface protein of interest and which comprises a selection marker.

The term “selection marker” as used herein may refer to a marker that can be used in flow cytometry, in particular in FACS. For FACS typically fluorescent markers are used. The skilled person is aware of different fluorescent markers that are useful for FACS, for example and without limitation fluorescent proteins expressed in the cell line, such as, without limitation, GFP, YFP or DsRed or derivatives thereof. In some embodiments, the first defined proportion of cells and the second defined proportion of may comprise the selection marker but the level of the selection marker may be different in the two proportions which allow distinguishing both proportions. For example, the selection marker may be present at moderate levels in the first defined proportion and may be present at high levels at the second defined proportion.

One aspect of the invention refers to a method for generating an antibody binding to a cell surface protein of interest, the method comprising the following steps:
(a) providing a mouse cell which does not express the endogenous form of the cell surface protein of interest but expresses an exogenous form of the cell surface protein of interest comprising at least one human segment;
(b) immunization of a non-human animal with the mouse cell line provided in step (a);
(c) generation of hybridomas from the immunized non-human animal of step (b);
(d) screening for an antibody that binds to the cell surface protein of interest by contacting the antibodies secreted by the hybridomas of step (c) with a mixture of human cells which does not express the endogenous form of the cell surface protein of interest comprising:
(i) a first defined proportion of the mixture of human cells which expresses the functional cell surface protein of interest; and
(ii) a second defined proportion of the mixture of human cells which does not express a functional cell surface protein of interest and which comprises a selection marker.
wherein the non-human animal is either a mouse or a rat.

One aspect of the invention refers to a method for generating an antibody binding to a cell surface protein of interest, the method comprising the following steps:
(a) providing a mouse cell which does not express the endogenous form of the cell surface protein of interest but expresses an exogenous form of the cell surface protein of interest comprising at least one human segment;
(b) immunization of a rat with the mouse cell line provided in step (a);
(c) generation of hybridomas from the immunized rat of step (b);
(d) screening for an antibody that binds to the cell surface protein of interest by contacting the antibodies secreted by the hybridomas of step (c) with a mixture of human cells which does not express the endogenous form of the cell surface protein of interest comprising:
(i) a first defined proportion of the mixture of human cells which expresses the functional cell surface protein of interest; and
(ii) a second defined proportion of the mixture of human cells which does not express a functional cell surface protein of interest and which comprises a selection marker.

Another aspect of the invention refers to a method for generating an antibody binding to a cell surface protein of interest, the method comprising the following steps:
(a) providing a mouse cell which does not express the endogenous form of the cell surface protein of interest but expresses an exogenous form of the cell surface protein of interest comprising at least one human segment;
(b) immunization of a non-human animal with the mouse cell line provided in step (a);
(c) generation of hybridomas from the immunized non-human animal of step (b);
(d) screening for an antibody that binds to the cell surface protein of interest by contacting the antibodies secreted by the hybridomas of step (c) with human cells which do not express the endogenous form of the cell surface protein of interest but express an exogenous form of the cell surface protein of interest comprising at least one human segment;
wherein the non-human animal is either a mouse or a rat.

As a non-limiting example, the generation of antibodies binding to a TCRs is shown.

Therefore, one embodiment relates to a method for generating an antibody binding to a TCR of interest, the method comprising the following steps:
(a) providing a non-human cell which does not express the endogenous form of the TCR of interest but expresses an exogenous form of the TCR of interest comprising at least one human segment;
(b) immunization of a non-human animal with the cell line provided in step (a);
(c) generation of hybridomas from the immunized non-human animal of step (b);
(d) screening for an antibody that binds to the surface protein of interest by contacting the antibodies secreted by the hybridomas of step (c) with human cells which do not express the endogenous form of the TCR of interest but express an exogenous form of the TCR of interest comprising at least one human segment.

The cell line provided in step (a) may be a mouse BW^{-/-} cell line.

The term “BW^{-/-} cell line” refers to a BW cell line, which was derived from the parental BW5147 thymoma that arose spontaneously in an AKR mouse (Lee N E and Davis M M., J Immunol. 1988 Mar. 1; 140(5):1665-75; Letourneur F., Malissen B., Eur J Immunol. 1989; 19(12):2269-2274) and does neither express the endogenous TCR α chain nor the endogenous TCR β chain. Since the surface expression of a TCR heterodimer is dependent on association with the CD3 protein complex the BW^{-/-} cell line was stably transduced to co-express human CD3 with GFP (BW^{-/-}-CD3-GFP; herein referred to simply as BW^{-/-}), enabling transduced cells to be easily identified. The presence of human CD3 allows these cells to express any human or mouse transgenic TCR at the cell surface.

The human cell line of step (d) may be a Jurkat cell^{-/-} line.

The terms “Jurkat^{-/-} ” and “Jurkat76^{-/-}” refer to a human Jurkat76^{-/-} cell line which is a variant of the original human TCL line that does not express human Vα and Vβ chains (Abraham R T, Weiss A., Nat Rev Immunol. 2004 April; 4(4):301-8). It has all remaining TCR-associated CD3 components necessary for transgenic TCR surface expression.

Another embodiment relates to a method for generating an antibody binding to a TCR of interest, the method comprising the following steps:
(a) providing a mouse BW^{-/-} cell line which expresses an exogenous form of the TCR of interest comprising at least one human segment;
(b) immunization of a non-human animal with the cell line provided in step (a);
(c) generation of hybridomas from the immunized non-human animal of step (b);
(d) screening for an antibody that binds to the surface protein of interest by contacting the antibodies secreted by the hybridomas of step (c) with Jurkat^{-/-} cells which do not express the endogenous form of the TCR of interest but express an exogenous form of the TCR of interest comprising at least one human segment.

A further embodiment relates to a method for generating an antibody binding to a TCR of interest, the method comprising the following steps:
(a) providing a mouse BW^{-/-} cell line which expresses an exogenous form of the TCR of interest comprising at least one human segment;
(b) immunization of a non-human animal with the cell line provided in step (a);
(c) generation of hybridomas from the immunized non-human animal of step (b);
(d) screening for an antibody that binds to the TCR of interest by contacting the antibodies secreted by the hybridomas of step (c) with a mixture of Jurkat^{-/-} cells which do not express the endogenous form of the TCR of interest comprising:
(i) a first defined proportion of the mixture of Jurkat^{-/-} cells which expresses the TCR of interest; and
(ii) a second defined proportion of the mixture of Jurkat^{-/-} cells which does not express a functional TCR of interest and which comprises a selection marker.

An additional embodiment relates to a method for generating an antibody binding to a TCR of interest, the method comprising the following steps:
(a) providing a mouse BW^{-/-} cell line which expresses an exogenous form of the TCR of interest comprising at least one human segment;
(b) immunization of a rat with the cell line provided in step (a);
(c) generation of hybridomas from the rat of step (b);
(d) screening for an antibody that binds to the TCR of interest by contacting the antibodies secreted by the hybridomas of step (c) with a mixture of Jurkat^{-/-} cells which do not express the endogenous form of the TCR of interest comprising:
(i) a first defined proportion of the mixture of Jurkat^{-/-} cells which expresses the TCR of interest; and
(ii) a second defined proportion of the mixture of Jurkat^{-/-} cells which does not express a TCR of interest and which comprises a selection marker.

An additional embodiment relates to a method for generating an antibody binding to a TCR of interest, the method comprising the following steps:
(a) providing a mouse cell which does not express the endogenous form of the TCR of interest but expresses an exogenous form of the TCR of interest comprising at least one human segment;
(b) immunization of a rat with the cell line provided in step (a);
(c) generation of hybridomas from the rat of step (b);
(d) screening for an antibody that binds to the surface protein of interest by contacting the antibodies secreted by the hybridomas of step (c) with a mixture with human cells which do not express the endogenous form of the TCR of interest comprising:
(i) a first defined proportion of the mixture of human cells which expresses the TCR of interest; and
(ii) a second defined proportion of the mixture of human cells which does not express TCR of interest and which comprises a selection marker.

In a specific embodiment the invention refers to a method for generating an antibody that binds to at least one TCR Vα chain or binds to at least one TCR Vβ chain, the method comprising the following steps:
(a) providing a non-human cell which does neither express the endogenous TCR α chain nor the endogenous TCR β chain and expresses an exogenous TCR α chain and an exogenous TCR β chain comprising a variable human TCR V α chain and variable human TCR β chain;
(b) immunization of a non-human animal with the cell line provided in step (a);
(c) generation of hybridomas from the immunized non-human animal of step (b);
(d) screening for an antibody that binds to at least one TCR Vα chain or binds to at least one TCR Vβ chain by contacting the antibodies secreted by the hybridomas of step (c) with a mixture of human cells which express neither the endogenous TCR α chain nor the endogenous TCR β chain comprising:
   (i) a first defined proportion of the mixture of human cells which comprises the TCR having the TCR chains that are expressed by the non-human cell provided in step (a),
   (ii) a second defined proportion of the mixture of human cells which does not comprise a TCR having TCR chains that are expressed by the non-human cell line provided in step (a) but comprises a TCR having TCR chains that are different to the TCR chains expressed by the non-human cell provided in step (a), and
   (iii) a third defined proportion of the mixture of human cells which does not comprise a functional TCR but comprises a selection marker.

In a specific embodiment the invention refers to a method for generating an antibody that binds to at least one T cell receptor variable alpha (TCR Vα) chain or binds to at least one T cell receptor variable beta (TCR Vβ) chain, the method comprising the following steps:
(a) providing a non-human cell which does neither express the endogenous TCR α chain nor the endogenous TCR β chain and expresses an exogenous TCR α chain and an exogenous TCR β chain comprising a variable human TCR V α chain and variable human TCR β chain;
(b) immunization of a non-human animal with the cell line provided in step (a);
(c) generation of hybridomas from the immunized non-human animal of step (b);
(d) screening for an antibody that binds to at least one TCR Vα chain or binds to at least one TCR Vβ chain by contacting the antibodies secreted by the hybridomas of step (c) with a mixture of human cells which express neither the endogenous TCR α chain nor the endogenous TCR β chain comprising:
   (i) a first defined proportion of the mixture of human cells which comprises the TCR having the TCR chains that are expressed by the non-human cell provided in step (a),
   (ii) a second defined proportion of the mixture of human cells which does not comprise a TCR having TCR chains that are expressed by the non-human cell line provided in step (a) but comprises a TCR having TCR chains that are different to the TCR chains expressed by the non-human cell provided in step (a), and
   (iii) a third defined proportion of the mixture of human cells which does not comprise a functional TCR but comprises a selection marker;
   wherein the non-human animal is mouse or rat and the non-human cell provided in step (a) is a mouse cell line.

Certain embodiments comprise a step of identifying an antibody that binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains, comprising the following steps:
(i) incubating human peripheral blood lymphocytes (PBL) with the antibody identified in step (d) as binding to at least one TCR Vα chain or binding to at least one TCR Vβ chain;
(ii) screening for cells that bind to the antibody by FACS sorting;
(iii) analysis of the TCR Vα chain repertoire or TCR Vβ chain repertoire of the cells that bind to the antibody of step (ii);
   wherein a TCR Vα chain repertoire or TCR Vβ chain repertoire comprising at least two different TCR Vα chains but less than all TCR Vα chains or at least two different TCR Vβ chains but less than all TCR Vβ chains indicates that the antibody binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains.

For example, one embodiment the invention refers to a method for generating an antibody that binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains, the method comprising the following steps:
(a) providing a non-human cell which does neither express the endogenous TCR α chain nor the endogenous TCR β chain but expresses an exogenous TCR α chain comprising a variable human TCR V α chain and an exogenous TCR β chain comprising a variable human TCR β chain;
(b) immunization of a non-human animal with the cell line provided in step (a);
(c) generation of hybridomas from the immunized non-human animal of step (b);
(d) screening for an antibody that binds to at least one TCR Vα chain or binds to at least one TCR Vβ chain by contacting the antibodies secreted by the hybridomas of step (c) with a mixture of human cells which express neither the endogenous TCR α chain nor the endogenous TCR β chain comprising:
   (i) a first defined proportion of the mixture of human cells which comprises the TCR having the TCR chains that are expressed by the non-human cell provided in step (a),
   (ii) a second defined proportion of the mixture of human cells which does not comprise a TCR having TCR chains that are expressed by the non-human cell line provided in step (a) but comprises a TCR having TCR chains that are different to the TCR chains expressed by the non-human cell provided in step (a), and
   (iii) a third defined proportion of the mixture of human cells which does not comprise a functional TCR but comprises a selection marker;
   wherein the non-human animal is mouse or rat and the non-human cell provided in step (a) is a mouse cell line.
(e) identifying an antibody that binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains, comprising the following steps:
   (i) incubating human peripheral blood lymphocytes (PBL) with the antibody identified in step (d) as binding to at least one TCR Vα chain or binding to at least one TCR Vβ chain;
   (ii) screening for cells that bind to the antibody by FACS sorting;
   (iii) analysis of the TCR Vα chain repertoire or TCR Vβ chain repertoire of the cells that bind to the antibody of step (ii);
   wherein a TCR Vα chain repertoire or TCR Vβ chain repertoire comprising different TCR Vα chains but less than all TCR Vα chains or at least two different TCR Vβ chains but less than all TCR Vβ chains indicates that the antibody binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains.

Another embodiment of the invention refers to a method for generating an antibody that binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains, the method comprising the following steps:
(a) providing a mouse cell which does neither express the endogenous TCR α chain nor the endogenous TCR β chain but expresses an exogenous TCR α chain comprising a variable human TCR V α chain and an exogenous TCR β chain comprising a variable human TCR β chain;
(b) immunization of a rat with the cell line provided in step (a);
(c) generation of hybridomas from the immunized rat of step (b);
(d) screening for an antibody that binds to at least one TCR Vα chain or binds to at least one TCR Vβ chain by contacting the antibodies secreted by the hybridomas of step (c) with a mixture of human cells which express neither the endogenous TCR α chain nor the endogenous TCR β chain comprising:
   (i) a first defined proportion of the mixture of human cells which comprises the TCR having the TCR chains that are expressed by the non-human cell provided in step (a),
   (ii) a second defined proportion of the mixture of human cells which does not comprise a TCR having TCR chains that are expressed by the non-human cell line provided in step (a) but comprises a TCR having TCR chains that are different to the TCR chains expressed by the non-human cell provided in step (a), and
   (iii) a third defined proportion of the mixture of human cells which does not comprise a functional TCR but comprises a selection marker;
   wherein the non-human animal is mouse or rat and the non-human cell provided in step (a) is a mouse cell line.
(e) identifying an antibody that binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains, comprising the following steps:
   (i) incubating human peripheral blood lymphocytes (PBL) with the antibody identified in step (d) as binding to at least one TCR Vα chain or binding to at least one TCR Vβ chain;
   (ii) screening for cells that bind to the antibody by FACS sorting;
   (iii) analysis of the TCR Vα chain repertoire or TCR Vβ chain repertoire of the cells that bind to the antibody of step (ii);
   wherein a TCR Vα chain repertoire or TCR Vβ chain repertoire comprising different TCR Vα chains but less than all TCR Vα chains or at least two different TCR Vβ chains but less than all TCR Vβ chains indicates that the antibody binds to a fraction of TCR Vα chains comprising at least two different TCR Vα chains but less than all TCR Vα chains or that binds to a fraction of TCR Vβ chains comprising at least two different TCR Vβ chains but less than all TCR Vβ chains.

The analysis of the TCR Vα chain repertoire or TCR Vβ chain repertoire may be carried out for example by PCR or by next generation sequencing methods. Methods for identifying the sequence of a nucleic acid are well known to those skilled in the art.

### TCR Library

In a further aspect the present application is concerned with a library for the expression of all functional TCR types comprising 45 TCR constructs each encoding one of the 45 different TCR α chains and 47 TCR constructs each encoding one of the 47 different TCR β chains, wherein each of the 45 TCR constructs encoding one of 45 different TCR α chain comprises the following building blocks:
one of the variable AV1 to AV45 segments, and
a constant AC segment; and
   wherein each of the 47 TCR constructs encoding one of 47 different TCR β chains comprises the following building blocks:
one of the variable BV1 to BV47 segments, and
a constant BC segment.

Certain embodiments refer to a library for the expression of all functional TCR types comprising 45 TCR constructs each encoding one of the 45 different TCR α chains and 47 TCR constructs each encoding one of the 47 different TCR β chains, wherein each of the 45 TCR constructs encoding one of 45 different TCR α chain comprises the following building blocks:
(i) one of the variable AV1 to AV45 segments;
(ii) a linker sequence specific for the A segment; and
(iii) a constant AC segment; and
   wherein each of the 47 TCR constructs encoding one of 47 different TCR β chains comprises:
(i) one of the variable BV1 to BV47 segments,
(ii) a linker sequence specific for the B segment, and
(iii) a constant BC segment.

The term “functional TCR types” refers to TCRs that are composed of TCR variable chains that are expressed on T cells. A “TCR receptor construct” refers to a nucleic acid sequence that encodes a TCR α chain or a TCR β chain.

The term “building block” as used herein refers to the elements of the TCR library and the expression system for expressing TCRs, such as the variable AV and AB segments, the constant AC and BC segments, the linker sequences and the backbone vectors.

The linker sequence specific for the A segment may be any sequence that will be considered by the skilled person in the art as useful for linking a variable AV segment with the constant AC segment. The linker may contain sequences that are useful for the recombination, such as, without limitation, one or several restriction sites or may contain sequences useful for modifying the TCR construct via cloning. Further, the linker may contain any AJ and or CDR3 sequence, so that the construct consisting of the (i) one variable AV segment, (ii) a linker sequence specific for the A segment and (iii) a constant AC segment encodes a functional TCR α chain. In a specific embodiment the linker sequence has a sequence which is at least 90% identical to the sequence set forth in SEQ ID No: 192 or which is at least 90% identical to the sequence set forth in SEQ ID No: 194. In a more specific embodiment the linker sequence has a sequence which set forth in SEQ ID No: 192 and in SEQ ID No: 194.

The linker sequence specific for the B segment may be any sequence that will be considered by the skilled person in the art as useful for linking a variable BV segment with the constant BC segment. The linker may contain one or several restriction sites or may contain sequences useful for modifying the TCR construct via cloning. Further, the linker may contain any BD, BJ and/or CDR3 sequence, so that the construct consisting of the (i) one variable BV segment, (ii) a linker sequence specific for the B segment and (iii) a constant BC segment encodes a functional TCR β chain. In a specific embodiment the linker sequence has a sequence which is at least 90% identical to the sequence set forth in SEQ ID No: 193 or which is at least 90% identical to the sequence set forth in SEQ ID No: 195. In a more specific embodiment the linker sequence has a sequence which set forth in SEQ ID No: 193 and in SEQ ID No: 195.

The AC segment and the BC segment may be murine, minimal-murinized, cysteine-engineered or wild-type human or a combination thereof.

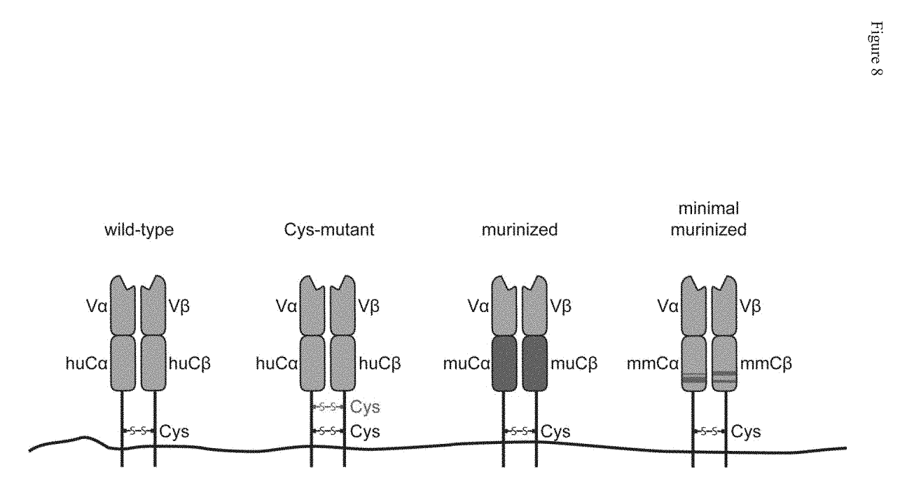

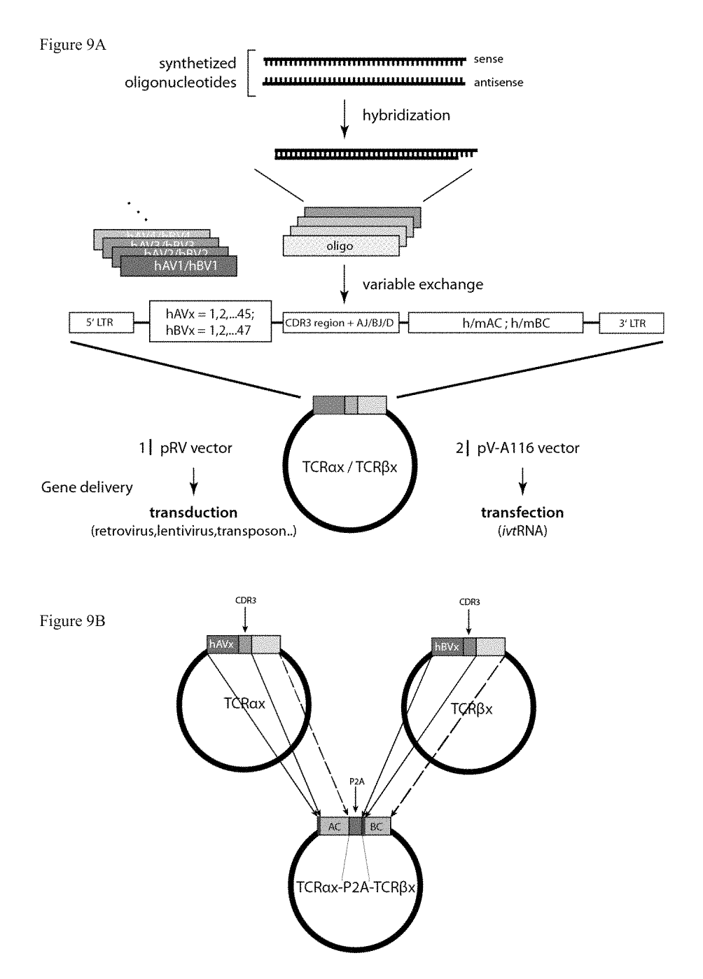

These modifications may improve pairing of the TCR α and TCR β chain. “cysteine-engineered” AC and BC segments encode for mutations of single amino acids to cysteines in each TCR chain and lead to formation of an additional disulfide bond connecting the C regions of the TCR α and TCR β chain (Cohen, C. J., Li, Y. F., El-Gamil, M., Robbins, P. F., Rosenberg, S. a, & Morgan, R. a. (2007), Cancer Research, 67(8), 3898-903.). This reduces mixed TCR pairing and enhances the functionality of TCR gene-modified T cells. Therefore, human TCRs are equipped with murine C regions lead to a more stable expression of the TCRs, this so called “murinization” increases the cell surface expression of these hybrid TCRs compared with wild-type (wt) human TCRs and results in a higher functional avidity of T cells modified with different TCRs (Cohen, C. J., Zhao, Y., Zheng, Z., Rosenberg, S. a, & Morgan, R. a. (2006). Cancer Research, 66(17), 8878-86). Alternatively the AC and the BC segments can be minimal-murinized, i.e. the critical amino acids within the C regions of the murine TCR α and 3 chain that ensure TCR cell surface expression comparable to full replacement of human C regions are exchanged (Sommermeyer, D., & Uckert, W. (2010); Journal of Immunology (Baltimore, Md.: 1950), 184(11), 6223-31.). See also FIG. 8. In a preferred embodiment, the AC segment and the BC segment are murine or human.

In another embodiment the variable AV segments and variable BV segments are human or murine. In a preferred embodiment the variable AV segments and variable BV segments are human. In an even more preferred embodiment the AC segment and the BC segment are murine and the variable AV segments and variable BV segments are human.

In particular, if the TCRs are used for non-therapeutic use, such as the generation of TCR specific antibodies, it is advantageous that the AC segment and the BC segment be murine and the variable AV segments and variable BV segments be human.

In another preferred embodiment the AC segment and the BC segment are human and the variable AV segments and the variable BV segments are human.

In particular, if the TCRs that are produced by the library as described herein are used for therapy, it is advantageous that the AC segment and the BC segment be human and the variable AV segments and the variable BV segments be human.

The sequence of the TCR constructs may be modified, e.g., without limitation, it may be codon optimized or further restriction sites may be inserted for example by exchange of nucleotides. In preferred embodiments, the sequence of the TCR constructs is codon optimized for the expression in mammalian cells, preferably in human cells. Alternatively the sequence of the TCR construct may not be modified.

For example, SEQ ID No: 1 is a modified version of nucleotide sequence SEQ ID No: 2 encoding the human constant α region, since SEQ ID No: 1 further contains a DraIII restriction site. Another example is SEQ ID No: 4 which is a modified version of nucleotide sequence SEQ ID No: 5 encoding the human constant 1 region, as it further contains a BstEII restriction site.

The building blocks of the TCR construct are constructed so that they can be easily exchanged, e.g. by a single cloning step. That means that the elements contain combination sites that are compatible, i.e. all AV segments comprise combination sites at the 5′-end that can be combined with the combination sites of the 3′-end of the backbone vectors and further comprise combination sites at their 3′-end that can be combined with the linker sequence specific for the A segment. In addition, all AC segments comprise combination sites at their 5′-end that can be combined with the linker sequence specific for the A segment and further comprise combination sites at their 3′-end that can be combined with the 5′-end of the backbone vector. Thus, the linker sequences specific for the A segment comprise combination sites at their 5′-end that can be combined with the combination site of the 3′-end of the AV segments and further comprise combination sites at their 3′-end that can be combined with the combination site of the 5′-end of the AC segments. Further, all BV segments comprise combination sites at the 5′-end that can be combined with the combination site of the 3′-end of the backbone vectors and further comprise combination sites at their 3′-end that can be combined with the linker sequence specific for the B segment. In addition, all BC segments comprise combination sites at their 5′-end that can be combined with the linker sequence specific for the B segment and further comprise combination sites at their 3′-end that can be combined with the 5′-end of the backbone vector. Thus, the linker sequences specific for the B segment comprise combination sites at their 5′-end that can be combined with the combination site of the 3′-end of the BV segments and further comprise combination sites at their 3′-end that can be combined with the combination site of the 5′-end of the BC segments.

The term “combination site” as used herein refers to any sequence that is useful for cloning in order to exchange sequences in a vector, such as, without limitation, restriction sites, recombination sequences or homology regions for seamless cloning techniques.

For example, all AV segments comprise restriction sites at the 5′-end that can be combined with the restriction site of the 3′-end of the backbone vectors and further comprise restriction sites at their 3′-end that can be combined with the linker sequence specific for the A segment. In addition, all AC segments comprise restriction sites at their 5′-end that can be combined with the linker sequence specific for the A segment and further comprise restriction sites at their 3′-end that can be combined with the 5′-end of the backbone vector. Thus, the linker sequences specific for the A segment comprise restriction sites at their 5′-end that can be combined with the restriction site of the 3′-end of the AV segments and further comprise restriction sites at their 3′-end that can be combined with the restriction site of the 5′-end of the AC segments.

Further, all BV segments comprise restriction sites at the 5′-end that can be combined with the restriction site of the 3′-end of the backbone vectors and further comprise restriction sites at their 3′-end that can be combined with the linker sequence specific for the B segment. In addition, all BC segments comprise restriction sites at their 5′-end that can be combined with the linker sequence specific for the B segment and further comprise restriction sites at their 3′-end that can be combined with the 5′-end of the backbone vector. Thus, the linker sequences specific for the B segment comprise restriction sites at their 5′-end that can be combined with the restriction site of the 3′-end of the BV segments and further comprise restriction sites at their 3′-end that can be combined with the restriction site of the 5′-end of the BC segments.

In certain embodiments the library may contain AV segments of different types, such as murine, minimal-murinized, cysteine-engineered or wild-type human, which comprise the same restriction sites at their 3′-end and their 5′-end, so that they can be easily exchanged. Accordingly, the library may contain BV segments of different types, such as murine, minimal-murinized, cysteine-engineered or wild-type human which comprise the same restriction sites at their 3′-end and their 5′-end, so that they can be easily exchanged.

In certain embodiments the library may contain AC segments of different types, such as murine, minimal-murinized, cysteine-engineered or wild-type human, which comprise the same restriction sites at their 3′-end and their 5′-end, so that they can be easily exchanged. Accordingly, the library may contain BC segments of different types, such as murine, minimal-murinized, cysteine-engineered or wild-type human which comprise the same restriction sites at their 3′-end and their 5′-end, so that they can be easily exchanged.

In certain embodiments, the variable AV segment is preceded by a NotI and/or AgeI restriction site and followed by a FspI restriction site.

In certain embodiments, the linker sequence specific for the A segment is preceded by a FspI restriction site and followed by a DraIII restriction site. In certain embodiments, the linker sequence specific for the A segment is preceded by a FspI restriction site and followed by a BspEI and/or a DraIII restriction site.

In certain embodiments, the linker sequence specific for the A segment is preceded by a FspI restriction site and followed by a BspEI restriction site.

In certain embodiments, the constant AC segment is preceded by a BspEI and/or DraIII restriction site and followed by MluI and/or ClaI and/or EcoRI restriction site.

In certain embodiments, the constant AC segment is preceded by a BspEI restriction site and followed by MluI and/or ClaI and/or EcoRI restriction site.

In specific embodiments, the variable AV segment is preceded by a NotI and/or AgeI restriction site and followed by a FspI restriction site. The linker sequence specific for the A segment is preceded by a FspI restriction site and followed by a BspEI and/or a DraIII restriction site. The constant AC segment is preceded by a BspEI and/or DraIII restriction site and followed by MluI and/or ClaI and/or EcoRI restriction site.

In certain embodiments, the variable BV segment is preceded by a NotI and/or AgeI restriction site and followed by a FspI restriction site.

In certain embodiments, the linker sequence specific for the B segment is preceded by a FspI restriction site and followed by a BstEII restriction site.

In certain embodiments, the constant BC segment is preceded by a BspEII restriction site and followed by MluI, ClaI and EcoRI restriction site.

In certain embodiments, the constant BC segment is preceded by a BspEII restriction site and followed by a EcoRI restriction site.

In specific embodiments, the variable BV segment is preceded by a NotI and/or AgeI restriction site and followed by a FspI restriction site. The linker sequence specific for the B segment is preceded by a FspI restriction site and followed by a BstEII restriction site. The constant BC segment is preceded by a BspEII restriction site and followed by MluI, ClaI and EcoRI restriction site.

Therefore, the variable segment, the linker sequence and the C segment can be replaced in a single cloning step. In addition the unique design of the restriction sites of the TCR constructs and the backbone vectors allows not only efficient exchange of the variable and the constant chains of the TCR and its CDR3 regions but also facilitates easy switching between the vectors for ivtRNA production and/or viral transfection.

A specific embodiment thus relates to a library for the expression of all functional TCR types comprising 45 TCR constructs, each encoding one of the 45 different TCR α chains and 47 TCR constructs each encoding one of the 47 different TCR β chains, wherein each of the 45 TCR constructs, encoding one of 45 different TCR α chain comprises:
(i) one of the variable AV1 to AV45 segments comprising a NotI and/or AgeI restriction site at the 5′-end and a FspI restriction site at the 3′-end
(ii) a linker sequence specific for the A segment comprising a FspI restriction site at the 5′-end a BspEI restriction site at the 3′ end, and
(iii) a constant AC segment comprising a BspEI and/or a DraIII restriction site at the 5′-end and a MluI and/or ClaI and/or EcoRI restriction site at the 3′-end; and
wherein each of the 47 TCR constructs encoding one of 47 different TCR β chains comprises:
(i) one of the variable BV1 to BV47 segments comprising a NotI and/or AgeI restriction site at the 5′-end and a FspI-restriction site at the 3′-end,
(ii) a linker sequence specific for the B segment comprising a FspI restriction site at the 5′-end and a BstEII restriction site at the 3′-end, and
(iii) a constant BC segment comprising a BspEII restriction site at the 5′-end and followed by MluI, ClaI and EcoRI restriction site at the 3′ end.

Accordingly in the expression systems for the expression of TCRs described herein the backbone vectors comprise compatible combination sites for the introduction of the library constructs. In a specific embodiment, the expression systems for the expression of TCRs described herein the backbone vectors comprise compatible restriction sites for the introduction of the library constructs.

For example, the AC segment may have a sequence which is at least 90% identical to the sequences set forth in SEQ ID NOs: 1, 2 or 6 and the BC segment may have a sequence which is at least 90% identical to the sequences set forth in SEQ ID NOs: 3, 4, 5 or 7. Particularly, the AC segment may have a sequence which is set forth in SEQ ID Nos: 1, 2 or 6 and the BC segment may have a sequence which is set forth in SEQ ID Nos: 3, 4, 5 or 7.

The variable AV segments AVseg1 to AVseg45 may have sequences which are at least 90% identical to the sequences set forth in SEQ ID No: 8 to SEQ ID No: 52 and the variable BV segments BV1 to BV47 segments may have sequences which are at least 90% identical to the sequences set forth in SEQ ID No: 53 to SEQ ID No: 99. In particular, the variable AV1 to AV45 segments may have sequences which are set forth in SEQ ID No: 8 to SEQ ID No: 52 and the variable BV1 to BV47 segments may have sequences which are set forth in SEQ ID No: 53 to SEQ ID No: 99.

The TCR constructs are integrated into at least one backbone vector.

The term “vector” as used herein is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. The backbone vector may be a circular or linear nucleic acid molecule to which an insert sequence can be integrated so as to bring about replication of the insert sequence. The vector may comprise any of a number of vector elements, such as those described below. The vector may be produced using a combination of in vitro and in vivo methods such as those described in Sambrook, J., et al., “Molecular Cloning: A Laboratory Manual,” which is incorporated herein by reference. Representative examples of vectors include, but are not limited to, in vitro transcription mRNA (ivtRNA) backbone vectors, transposon vectors (e.g. sleeping beauty transposon system), adenoviral backbone vectors, retroviral backbone vectors, lentiviral backbone vectors including next generation SIN retroviral or lentiviral vectors.

The vector may also comprise an insert site, which may be used to clone a nucleic acid. The insert site may be the recognition site of an endonuclease such as a Type I, II or III restriction enzyme, a homing endonuclease, or a nicking enzyme. The insert site may also be a specific site for homologous recombination. The insert site may be present in the vector only at the insert site. In certain circumstances, it may be desirable to remove other insert sites from the vector. For example, when the insert site is the recognition site for a restriction enzyme, it may be desirable to remove other such recognition sites from the chromosome.

Representative examples of Type I restriction enzymes include, but are not limited to, CfrAI, Eco377I, Eco394I, Eco585I, Eco646I, Eco777I, Eco826I, Eco851I, Eco912I, EcoAI, EcoBI, EcoDI, EcoDR2, EcoDR3, EcoDXXI, EcoEI, EcoKI, EcoprrI, EcoR124I, EcoR124II, EcoRD2, EcoRD3, HindI, KpnAI, KpnBI, NgoAV, StyLTIII, StySBLI, StySEAI, StySGI, StySJI, StySKI, StySPI and StySQI. Representative examples of Type III restriction enzymes include, but are not limited to, EcoP15I, EcoPI, HinfIII and StyLTI. Representative examples of Type II restriction enzymes include, but are not limited to, AarI, AatII, AccI, AceIII, AciI, AclI, AcyI, AflI, AflIII, AgeI, AhaIII, AjuI, AlfI, AloI, AluI, AlwFI, AlwNI, ApaBI, ApaI, ApaLI, ApoI, AscI, AspCNI, AsuI, AsuII, AvaI, AvaII, AvaIII, AvrII, BaeI, Ball, BamHI, BbvCI, BbvI, BbvII, BccI, Bce83I, BcefI, BcgI, BciVI, BelI, BdaI, BetI, BfiI, BglI, BglII, BinI, BmgI, BplI, Bpu10I, BsaAI, BsaBI, BsaXI, BsbI, BscGI, BseMII, BsePI, BseRI, BseSI, BseYI, BsgI, BsiI, BsiYI, BsmAI, BsmI, Bspl407I, Bsp24I, BspGI, BspHI, BspLU11I, BspMI, BspMII, BspNCI, BsrBI, BsrDI, BsrI, BstEII, BstXI, BtgZI, BtrI, BtsI, Cac8I, CauII, CdiI, Cfr10I, CfrI, CjeI, CjeNII, CjePI, ClaI, CspCI, CstMI, CviJI, CviRI, DdeI, DpnI, DraII, DraIII, DrdI, DrdII, DsaI, Eam11051, EciI, Eco31I, Eco47III, Eco57I, Eco57MI, EcoNI, EcoRI, EcoRII, EcoRV, Esp3I, EspI, FaII, FauI, FinI, Fnu4HI, FnuDII, FokI, FseI, FspI, GdiII, GsuI, HaeI, HaeII, HaeIII, HaeIV, HgaI, HgiAI, HgiCI, HgiEII, HgiJII, HhaI, Hin4I, Hin4II, HindII, HindIII, Hinfi, HpaI, HpaII, HphI, Hpy178III, Hpy188I, Hpy99I, KpnI, Ksp632I, MaeI, MaeII, MaeIII, MboI, MboII, McrI, MfeI, MjaIV, MluI, MmeI, MnII, MseI, MsII, MstI, MwoI, NaeI, NarI, NcoI, NdeI, NheI, NlaIII, NlaIV, NotI, NruI, NspBII, NspI, OliI, PacI, PasI, Pfl1108I, PflMI, PfoI, PleI, PmaCI, PmeI, PpiI, PpuMI, PshAI, PsiI, PspXI, PsrI, PstI, PvuI, PvuII, RleAI, RsaI, RsrII, SacI, SacII, SaII, SanDI, SapI, SauI, ScaI, ScrFI, SduI, SecI, SexAI, SfaNI, SfeI, Sfi, SgfI, SgrAI, SgrDI, SimI, SmaI, SmII, SnaBI, SnaI, SpeI, SphI, SpII, SrfI, Sse232I, Sse8387I, Sse8647I, SsmI, SspI, Sth132I, StuI, StyI, SwaI, TaqI, TaqII, TatI, TauI, TfiI, TseI, TsoI, Tsp45I, Tsp4CI, TspDTI, TspEI, TspGWI, TspRI, TssI, TstI, TsuI, Tth1111I, Tth111II, UbaF10I, UbaF9I, UbaPI, VspI, XbaI, XcmI, XhoI, XhoII, XmaIII and XmnI. Representative examples of homing endonucleases include, but are not limited to, F-SceI, F-SceII, F-SuvI, F-TevI, F-TevII, F-TfII, F-TflII, F-TflIV (also known as HegA), H-DreI, I-AmaI, I-AniI, I-BasI, I-BmoI, I-CeuI, I-CeuAIIP, I-ChuI, I-CmoeI, I-CpaI, I-CpaII, I-CreI, I-CrepsbIP, I-CrepsbIIP, I-CrepsbIIIP, I-CrepsbIVP, I-CsmI, I-CvuI, I-CvuAIP, I-DdiI, I-DirI, I-DmoI, I-HmuI, I-HmuII, I-HspNIP, I-LlaI, I-MsoI, I-NaaI, I-NanI, I-NclIP, I-NgrIP, I-NitI, I-NjaI, I-Nsp236IP, I-PakI, I-PbolP, I-PculP, I-PcuAI, I-PcuVI, I-PgrIP, I-PobIP, I-PogI, I-PorI, I-PorlIP, I-PpbIP, I-PpoI, I-ScaI, I-SceI, I-SceII, I-SceIII, I-SceIV, I-SceV, I-SceVI, I-SceVII, I-SneIP, I-SpomI, I-SquIP, I-Ssp6803I, I-SthPhiJP, I-SthPhiST3P, I-SthPhiS3bP, I-TevI, I-TevII, I-TevIII, I-Tsp061I, I-TwoI, I-UarHGPA1P, I-VinIP, I-ZbiIP, PI-MgaI, PI-MtuI, PI-MtuHIP, PI-MtuHIIP, PI-PabI, PI-PabII, PI-PfuI, PI-PfuII, PI-PkoI, PI-PkoII, PI-PspI, PI-Rma43812IP, PI-ScaI, PI-SceI, PI-TfuI, PI-TfuII, PI-ThyI, PI-TliI, PI-TliII and PI-ZbaI. Other possible types of endonucleases are enzymes characterized by the complexity of their recognition sites. A representative example of such an enzyme is FseI.

The vector may comprise a plurality of insert sites and the insert sites may be clustered as part of a multiple cloning site. The vector may also comprise more than one multiple cloning sites, which may be identical.

The constructs may be integrated into the backbone vectors by the cloning techniques known to the skilled person. These include use of Type I, Type II, Type IIS and Type IIG restriction enzyme based cloning approaches, use of recombination based cloning approaches such as Gateway® cloning (Life technologies, ThermoFisher), use of homology based cloning approaches such as Gibson Assembly® (NEB), GeneArt® (Life technologies, ThermoFisher) or In-Fusion® system (Clonetech) seamless cloning.

In a particular embodiment the backbone is an ivtRNA backbone vector or retroviral backbone vector.

The term “ivtRNA backbone vector” refers to any vector that can be used for in vitro transcription of RNA. ivtRNA backbone vectors contemplated for use in the invention include those comprising at a T7, a T3 and/or a sp6 promotor. Such vectors are well known to ordinary skill in the art. In one embodiment the ivtRNA backbone vector comprises a T7 and/or a sp6 promotor. Further the ivtRNA backbone vector may comprise at least one RNA stabilizing sequence, such as, without limitation a poly-adenine tail. The poly-adenine tails may comprise at least 40 adenines, at least 60 adenines, at least 80 adenines, at least 90 adenines, at least 100 adenines, at least 110 adenines.

As used herein, the term “retroviral backbone vector” refers to any vector that can be used for integration of a desired DNA construct into the host genome of a eukaryotic cell. The skilled person is aware of such vectors. A non-limiting example of a vector contemplated for use in the present invention is the MP71 retroviral backbone vector (Schambach A, Wodrich H, Hildinger M, Bohne J, Krausslich H G, Baum C., Mol Ther. 2000 November; 2(5):435-45; Hildinger M, Abel K L, Ostertag W, Baum C., J Virol. 1999 May; 73(5):4083-9). Receiver plasmids (pR) containing candidate DNA constructs are used for virus production. Retroviruses carrying the transgenes are subsequently utilized for transduction of target cells. Transduced cells permanently expressing the transgenic protein can easily be produced in large numbers. The skilled person is aware that a retroviral backbone vector may comprise elements such as long terminal repeat (LTR) sequences. The design of retroviral backbone vectors is known to those of ordinary skill in the art and is described in the pertinent texts and literature (e.g. “Retroviruses”, Coffin J M, et al. eds.; 1997).

Preferably, the replacement of the linker sequence specific for the A segment by a CDR3A sequence and AJ sequence results in a construct encoding a functional TCR α chain and replacement of the linker sequence specific for the A segment by a CDR3B sequence, a BD and BJ region results in a construct encoding a functional TCR β chain.

In a preferred embodiment, the CDR3A sequence and the AJ sequences, the CDR3 sequence, the BD and BJ region are contained in an oligonucleotide. Thereby, the library described herein allows the efficient generation of TCRs of any specificity by the insertion of any CDR3 region via an oligonucleotide.

In certain embodiments, the ivtRNA backbone vector has a sequence which is at least 90% identical to the sequence set forth in SEQ ID No: 196. In particular embodiments, the ivtRNA backbone vector has a sequence which is set forth in SEQ ID No: 196. In other embodiments, the retroviral backbone vector has a sequence which is at least 90% identical to the sequence set forth in SEQ ID No: 200. In particular embodiments, the retroviral backbone vector has a sequence which is set forth in SEQ ID No: 200.

Preferably, in the TCR construct encoding one TCR α chain and one TCR β chain, the sequence encoding one TCR α chain and the sequence encoding one TCR β chain are linked by elements that allow the expression of more than one protein from a vector. Such exemplary elements include without limitation internal ribosome entry sites (IRES) or ribosomal skipping elements. The ribosomal skipping element allows the stoichiometric production of the proteins that are encoded by the sequences flanking the element. The sequence prevents the ribosome form covalently linking a new inserted amino acid and let the ribosome continue translation resulting in a co-translational cleavage of the polyprotein. A preferred ribosomal skipping element is the P2A element.

Another aspect of the invention refers to an expression system for the expression of TCRs comprising
a library comprising 45 TCR constructs each encoding one of the 45 different variable TCR α chains and 47 TCR constructs each encoding one of the 47 different variable TCR β chains,
wherein each 45 TCR constructs encoding one of 45 different variable TCR α chain comprises:
   (i) one of the variable AV segments AVseg1 to AVseg45;
   (ii) a linker sequence specific for the A segment;
wherein each 47 TCR constructs encoding one of 47 different variable TCR β chain comprises:
   (i) one of the variable BV segments BVseg1 to BVseg47;
   (ii) a linker sequence specific for the B segment; and
at least one ivtRNA backbone vector selected from the group consisting of:
   (i) ivtRNA backbone vector comprising a AC segment
   (ii) ivtRNA backbone vector comprising a BC segment
   (iii) ivtRNA backbone vector comprising a AC and a BC segment; and/or
at least one retroviral backbone vector selected from the group consisting of:
   (iv) retroviral backbone vector comprising a AC segment
   (v) retroviral backbone vector comprising a BC segment
   (vi) retroviral backbone vector comprising a AC and a BC segment.

In certain embodiments of the invention, the expression system as described above, further comprises at least one lentiviral backbone vector selected from the group consisting of:
(vii) lentiviral backbone vector comprising a AC segment
(viii) lentiviral backbone vector comprising a BC segment
(ix) lentiviral backbone vector comprising a AC and a BC segment.

In certain embodiments, the ivtRNA backbone vector comprising a AC segment has a sequence which is at least 90% identical to the sequence set forth in SEQ ID NO: 197 and/or the ivtRNA backbone vector comprising a BC segment has a sequence which is at least 90% identical to the sequence set forth in SEQ ID NO: 198 and/or the ivtRNA backbone vector comprising a AC and a BC segment has a sequence which is at least 90% identical to the sequence set forth in SEQ ID NO: 199. In particular embodiments, the ivtRNA backbone vector comprising a AC segment has a sequence which is set forth in SEQ ID NO: 197 and/or the ivtRNA backbone vector comprising a BC segment has a sequence which is set forth in SEQ ID NO: 198 and/or the ivtRNA backbone vector comprising a AC and a BC segment has a sequence set forth in SEQ ID NO: 199.

In other embodiments, the retroviral backbone vector comprising a AC segment has a sequence which is at least 90% identical to the sequence set forth in SEQ ID No: 201 and/or the retroviral backbone vector comprising a BC segment has a sequence which is at least 90% identical to the sequence set forth in SEQ ID No: 202 and/or the retroviral backbone vector comprising a AC and a BC segment has a sequence which is at least 90% identical to the sequence set forth in SEQ ID No: 203. In particular embodiments, the retroviral backbone vector comprising a AC segment has a sequence which is set forth in SEQ ID No: 201 and/or the retroviral backbone vector comprising a BC segment has a sequence which is set forth in SEQ ID No: 202 and/or the retroviral backbone vector comprising a AC and a BC segment has a sequence set forth in SEQ ID No: 203.

An additional aspect of the invention refers to an expression system for the expression of TCRs comprising
a library comprising 45 TCR constructs each encoding one of the 45 different variable TCR α chains and 47 TCR constructs each encoding one of the 47 different variable TCR β chains,
wherein each of the 45 TCR constructs encoding one of 45 different variable TCR α chain comprises one of the variable AV segments AVseg 1 to AVseg 45;
wherein each of the 47 TCR constructs encoding one of 47 different variable TCR 13 chain comprises one of the variable BV segments BVseg 1 to BVseg 47; and
at least one ivtRNA backbone vector selected from the group consisting of:
   (i) ivtRNA backbone vector comprising a AC segment and a linker sequence specific for the A segment,
   (ii) ivtRNA backbone vector comprising a BC segment and a linker sequence specific for the B segment,
   (iii) ivtRNA backbone vector comprising a AC segment, a linker sequence specific for the A segment, a BC segment and a linker sequence specific for the B segment, and/or
at least one retroviral backbone vector selected from the group consisting of:
   (iv) retroviral backbone vector comprising a AC segment and linker sequence specific for the A segment,
   (v) retroviral backbone vector comprising a BC segment and linker sequence specific for the B segment,
   (vi) retroviral backbone vector comprising a AC segment, a linker sequence specific for the A segment, a BC segment and a linker sequence specific for the B segment.

In one embodiment this expression system further comprises at least one lentiviral backbone vector selected from the group consisting of:
(vii) lentiviral backbone vector comprising a AC segment,
(viii) lentiviral backbone vector comprising a BC segment,
(ix) lentiviral backbone vector comprising a AC and a BC segment.

The skilled person understands that the invention also contemplates expression systems as described above which comprise ivtRNA backbone vectors (i) to (iii) and retroviral backbone vectors (iv) to (vi). Further, it is clear that the above described expression systems may comprise ivtRNA backbone vectors (i) to (iii), retroviral backbone vectors (iv) to (vi) and lentiviral backbone vectors (vii) to (ix).

A further aspect relates to a library of cell clones expressing TCRs comprising a population of cell clones expressing 45 different TCR α chains and a population of cell clones expressing 47 different TCR β chains,

wherein each of the cell clones expressing different TCR α chains comprises one of the 45 TCR constructs encoding one of 45 different TCR α chains as described herein and one TCR construct encoding a TCR β chain; and
wherein each of the cell clones expressing different TCR β chains comprises one of the 47 TCR constructs encoding one of 47 different TCR β chains as described herein and one TCR construct encoding a TCR α chain.

Certain embodiments relate to a library of cell clones expressing TCRs comprising a population of cell clones expressing 45 different TCR α chains and a population of cell clones expressing 47 different TCR β chains,

wherein each of the cell clones expressing different TCR α chains comprises one of the 45 TCR constructs encoding one of 45 different TCR α chains according to claim 1 and one TCR construct encoding a TCR β chain; and
wherein each of the cell clones expressing different TCR β chains comprises one of the 47 TCR constructs encoding one of 47 different TCR β chains according to claim 1 and one TCR construct encoding a TCR α chain;
wherein the cell clones do neither express the endogenous TCR α chain nor the endogenous TCR β chain.

In certain embodiments the cell clones are of the BW^{-/-} cell line and/or the Jurkat^{-/-} cell line.

The term “BW^{-/-} cell line” refers to a BW cell line, which was derived from the parental BW5147 thymoma that arose spontaneously in an AKR mouse (Lee N E and Davis M M., J Immunol. 1988 Mar. 1; 140(5):1665-75; Letourneur F., Malissen B., Eur J Immunol. 1989; 19(12):2269-2274) and does neither express the endogenous TCR α chain nor the endogenous TCR β chain. Since the surface expression of a TCR heterodimer is dependent on association with the CD3 protein complex the BW^{-/-} cell line was stably transduced to co-express human CD3 with GFP (BW^{-/-}-CD3-GFP) (hereafter referred to simply as BW^{-/-}), enabling transduced cells to be easily identified. The presence of human CD3 allows these cells to express any human or mouse transgenic TCR at the cell surface after successful co-transduction with selected AV- and BV-encoding RVs.

The terms “Jurkat^{-/-}” and “Jurkat76^{-/-}” refer to a human Jurkat76^{-/-} cell line which is a variant of the original human TCL line that does not express human Vα and Vβ chains (Abraham R T, Weiss A., Nat Rev Immunol. 2004 April; 4(4):301-8). It has all remaining TCR-associated CD3 components necessary for transgenic TCR surface expression, after transduction with appropriate RVs of choice.

A further aspect of the invention relates to a library of TCR proteins comprising a population of TCR proteins comprising 45 different TCR α chains and a population of TCR proteins 47 comprising 47 different TCR β chains,
wherein each of the TCR proteins comprising different TCR α chains comprises one of the 45 different TCR α chains encoded by the TCR constructs according to claim 1 and a TCR β chains; and
wherein each of the TCR proteins comprising different TCR β chains comprises one of the 47 different TCRβ chains encoded by the TCR constructs according to claim 1 and a TCR α chains.

As already described, the TCR library can be used for the immunization of animals in order to generate polyclonal and monoclonal, preferably monoclonal antibodies. The TCR library can be used for the generation of pan-specific, cluster-specific and mono-specific antibodies. In a preferred embodiment, the TCR library can be used for the generation of cluster-specific antibodies. In particular, the library may be used for the immunization of animals for antibody production and the selection of TCR specific antibodies.

The library is constructed in a way that it can be specifically adapted to the needs of its application:

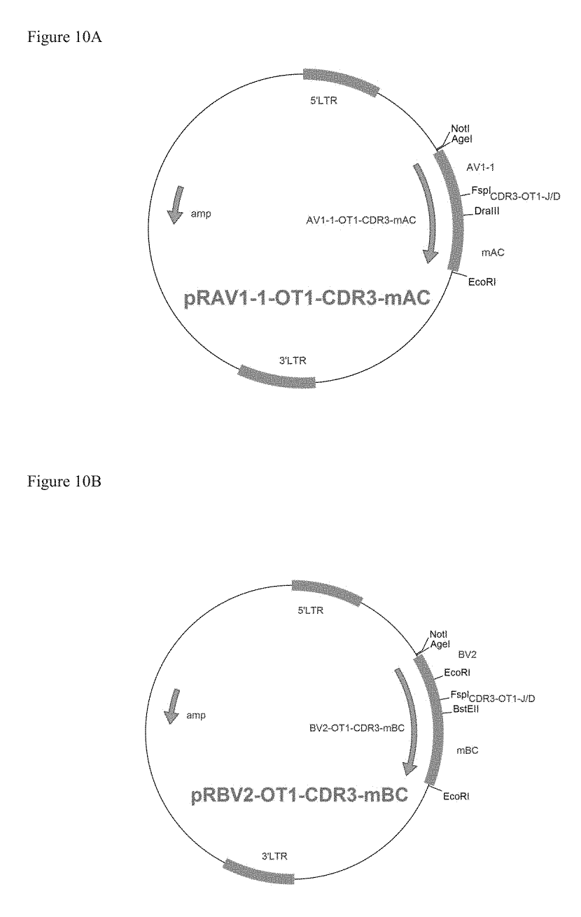

In particular, if the library is used for the generation of TCR-specific antibodies, a TCR construct coding for a TCR having mouse constant region, human variable regions and linker sequence may be used. More particular, if the library is used for the generation of TCR-specific antibodies, a TCR construct coding for a TCR having mouse constant region, human variable regions and a mouse linker sequence may be used. Preferably, the TCR construct is integrated into a retroviral backbone vector. Exemplary vectors that may be used for the generation of TCR-specific antibodies are shown in FIG. 10. FIG. 10A depicts a vector that may be used for the generation of an antibody specific for the human AV1-1 region. The sequence of this vector is set forth in SEQ ID NO: 204. An exemplary vector that may be used for the generation of an antibody specific for the human BV2 chain is shown in FIG. 10B and its sequence is set forth in SEQ ID NO: 205.

On the other hand, if the library is used for the construction of therapeutic TCRs, the TCR construct coding for a TCR having human constant regions and human variable regions is used and a CDR3 having the desired specificity is introduced by an oligonucleotide.

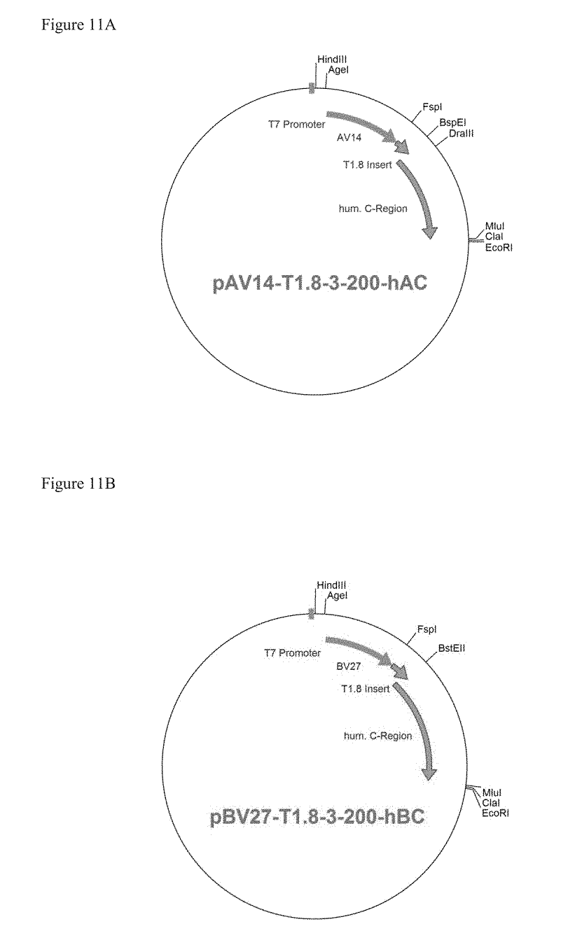

More specifically, for the production of the therapeutic TCR, the sequence of a candidate TCR is identified as described in detail in the section “Reengineering of an isolated TCR” for clone T1.8. Therefore, the specific sequence of the CDR3 region is sequenced. Further, the type of the variable region of the TCR α chain and of the variable region of the TCR β chain of the desired TCR is identified, either by PCR using primers specific for the variable TCR α chain and variable TCR β chain types, or by sequencing (For illustration, the sequence coding for the TCR α chain of the isolated T1.8 clone is set forth in SEQ ID No: 210 and the sequence coding for the TCR β chain of isolated clone T1.8 is depicted in SEQ ID No: 211). The TCR is then rebuilt by combining the AV and BV segments corresponding to the variable α chains and variable β chains identified for the desired TCR with the constant CA and CB segments respectively and replacing the linker sequence by the desired CDR 3 sequence using a synthesized oligonucleotide having this sequence. The sequences of the reengineered TCR are shown in FIG. 11. The vector map of the reengineered TCR α chain is shown in FIG. 11A and its sequence is depicted in SEQ ID No: 208. The vector map of the reengineered TCR β chain is shown in FIG. 11B and its sequence is depicted in SEQ ID No: 209.

The building blocks of the TCR library can also be generated by DNA synthesis. DNA synthesis methods are well known to skilled person in the art.

Further, the library as described herein can be used for synthetic display screens in order to generate antibodies such as phage display, yeast display, ribosomal display or cellular display screens. The skilled person in the art is aware of the diverse display screening techniques which include naive, immunized library and synthetic library.

The library as described herein can be used for the transient or stable expression of TCRs for their characterization and/or their use in therapy.

Another aspect of the application refers to a TCR receptor comprising a TCR α chain having a amino acid sequence which is at least 50%, at least 60%, at least 70%, at least 80%, at least 85%, at least 90%, at least 95%, at least 96%, at least 97%, at least 98%, at least 99% identical to SEQ ID No: 249 and a TCR β chain having an amino acid sequence which is at least 50%, at least 60%, at least 70%, at least 80%, at least 85%, at least 90%, at least 95%, at least 96%, at least 97%, at least 98%, at least 99% identical to SEQ ID No: 250.

Certain embodiments relate to a TCR receptor comprising a TCR α chain having the amino acid sequence of SEQ ID No: 249 and a TCR β chain comprising the amino acid sequence of SEQ ID No: 250.

Further, the application is related to a TCR receptor comprising a TCR α chain and a TCR 1 chain, wherein
the TCR α chain comprises a amino acid sequence which is at least 80% identical to SEQ ID No: 249 and comprises a CDR3 having the sequence of SEQ ID No: 245;
the TCR β chain comprises a amino acid sequence which is at least 80% identical to SEQ ID No: 250 and comprises a CDR3 having the sequence of SEQ ID No: 246.

Certain embodiments relate to a TCR receptor comprising TCR α chain and a TCR β chain, wherein
the TCR α chain comprises a amino acid sequence which is at least 50%, at least 60%, at least 70%, at least 80%, at least 85%, at least 90%, at least 95%, at least 96%, at least 97%, at least 98%, at least 99% identical to SEQ ID No: 249 and comprises a CDR3 having the sequence of SEQ ID No: 245;
the TCR β chain comprises amino acid sequence which is at least 50%, at least 60%, at least 70%, at least 80%, at least 85%, at least 90%, at least 95%, at least 96%, at least 97%, at least 98%, at least 99% identical to SEQ ID No: 250 and comprises a CDR3 having the sequence of SEQ ID No: 246.

Certain embodiments refer to a TCR receptor comprising a TCR α chain encoded by a nucleotide sequence which is at least 50%, at least 60%, at least 70%, at least 80%, at least 85%, at least 90%, at least 95%, at least 96%, at least 97%, at least 98%, at least 99% identical to SEQ ID No: 247 and a TCR β chain encoded by a nucleotide sequence which is at least 50%, at least 60%, at least 70%, at least 80%, at least 85%, at least 90%, at least 95%, at least 96%, at least 97%, at least 98%, at least 99% identical to SEQ ID No: 248.

Certain embodiments relate to a TCR receptor comprising a TCR α chain encoded by the nucleotide sequence SEQ ID No: 247 and a TCR β chain encoded by the nucleotide sequence SEQ ID No: 248.

Further, the application is related to a TCR receptor comprising a TCR α chain and a TCR 13 chain, wherein
the TCR α chain is encoded by a nucleotide sequence which is at least 80% identical to SEQ ID No: 247 and comprises a CDR3 region encoded by the nucleotide sequence set out in SEQ ID No: 243;
the TCR β chain is encoded by a nucleotide sequence which is at least 80% identical to SEQ ID No: 248 and comprises a CDR3 region encoded by the nucleotide sequence set out SEQ ID No: 244.

Certain embodiments relate to a TCR receptor comprising TCR α chain and a TCR β chain, wherein
the TCR α chain is encoded by a nucleotide sequence which is at least 50%, at least 60%, at least 70%, at least 80%, at least 85%, at least 90%, at least 95%, at least 96%, at least 97%, at least 98%, at least 99% identical to SEQ ID No: 247 and comprises a CDR3 region encoded by the nucleotide sequence set out in SEQ ID No: 243;
the TCR β chain is encoded by a nucleotide sequence which is at least 50%, at least 60%, at least 70%, at least 80%, at least 85%, at least 90%, at least 95%, at least 96%, at least 97%, at least 98%, at least 99% identical to SEQ ID No: 248 and comprises a CDR3 region encoded by the nucleotide sequence set out in SEQ ID No: 244.

It is clear to the skilled person that the present application also relates to nucleotide acid molecules coding for the TCRs as defined above.

Another aspect of the application relates to the TCRs as defined above for use as a medicament.

Thus, the present application also contemplates a pharmaceutical composition comprising the TCRs as defined above and a pharmaceutically acceptable carrier. Certain embodiments refer to the TCRs as defined above for use in treating a disease involving malignant cells expressing NY-ESO1. Thus, the application also refers to the TCRs as defined above for use in the treatment of cancer.

### Experiments

### Generation of TCR-Specific Immunogens

As heterodimeric proteins expressed in association with CD3, the native TCR is a highly conformation-dependent structure. This complex structure impacts strongly on the exposure of epitopes that can be used to distinguish different V regions.

In a first step each and every TCR Vα and Vβ chain in its native configuration are expressed on the surface of recipient cells. These cells serve as immunogens and as primary screening cells. The cellular immunogens are developed in three steps. First, vector libraries that encode all 45 AV gene segments and all 47 BV gene segments in the human TCR repertoire are generated. Next, vectors are selected from this library as needed to create retroviruses (RV) to transduce TCR-negative cell lines (Jurkat-76^{-/-}), to thereby generate cell lines with individually defined VαVβ heterodimers. Third, these TCR-transgenic cell lines are selected by flow cytometry for TCR surface expression and individual T cell clones showing stable, high surface expression are obtained. These T cell clones become part of a master cell library after expansion, validation of their specific AV and BV regions by PCR, and cyro conservation.

### TCR Vector Library

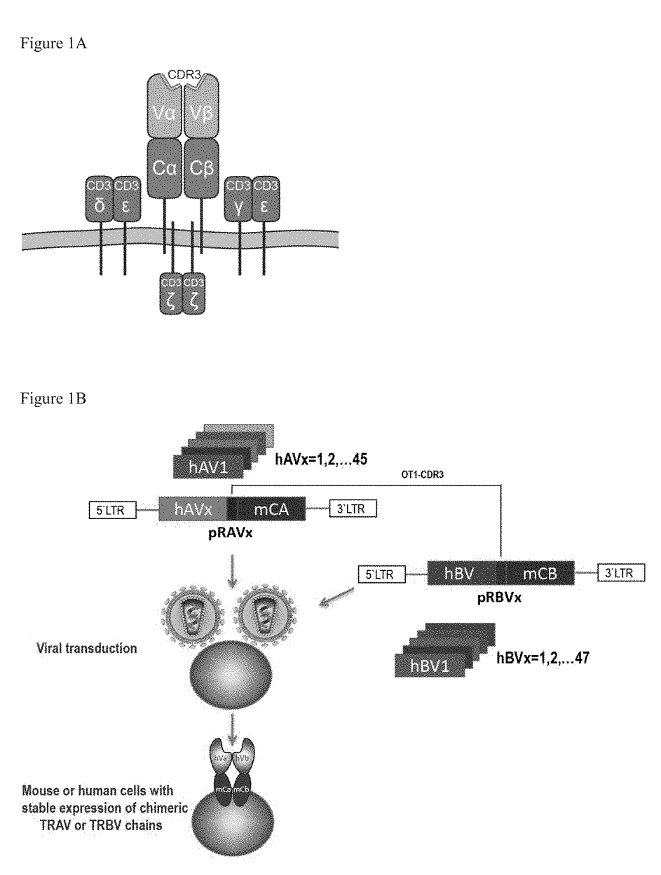

The modular TCR vector library was developed using the MP71 retroviral vector backbone (Schambach, 2000; Hildinger, 1999; FIG. 1B). The complete TCR vector library is composed of 92 different vectors, each containing one of the 45 separate AV or 47 separate BV variable regions. The vectors were designed in order to allow expression of different TCRs in the correct native conformation. A common CDR3 region was used in all vectors. This was derived from the OT-1 mouse T cell clone, which is specific for ovalbumin protein. Second, the human variable regions were combined with the respective mouse constant regions (mCA or mCB). The murine constant regions foster better pairing of human Vα and Vβ protein chains because they contain several charged amino acids, not present in human constant regions, which allow improved reciprocal protein interactions and better TCR heterodimer pairing and higher surface expression.

### Retrovirus Production and Cell Transduction

Individual pRAVx or pRBVx vectors are used to make corresponding RVs. An example demonstrating the capacity of chimeric human-mouse TCR chains encoded by these vectors to form heterodimers, with the appropriate confirmation at the cell surface, is shown in FIG. 1. Here a T cell line was transduced with a selected combination of pRAVx and pRBVx retroviruses. Surface expression of the transgenic TCR was assessed in flow cytometry using a hamster anti-mouse antibody specific for mCB. Surface expression of any TCR requires the fully correct formation of the α/β heterodimer and association with CD3. Thus, a TCR will only show surface expression if the heterodimer folds and pairs in a proper configuration. As seen in this case, a subpopulation of around 40% of cells expressed surface TCR after simultaneous transduction with two RVs. Upon selection of an individual clone, uniform stable expression was found on all T cells. In a similar manner, several other TCR heterodimers are expressed for which corresponding commercial Vβ-specific mabs are available. These were found to bind the mabs, demonstrating that the conformation was equivalent to that of human T cells for the epitopes recognized by the tested mabs (data not shown).

### Development of TCR Cell Libraries Expressing Chimeric TCRs

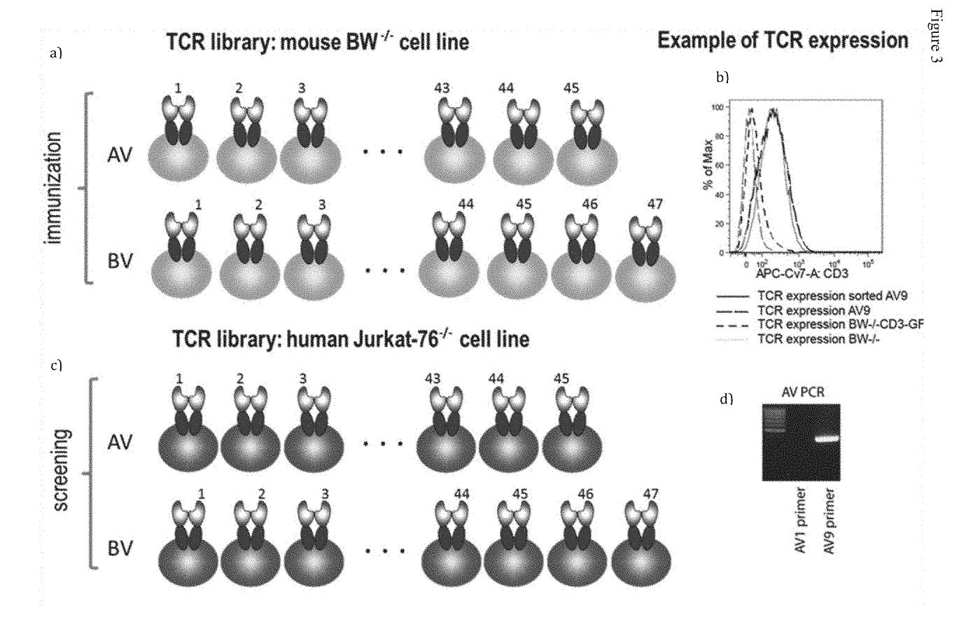

Two TCR cell libraries are developed using the respective AV and BV retroviral vector libraries. Upon retroviral transduction with selected RVs, cells were stained with an antibody that is useful for detecting the expression of a functional TCR and positive cells were sorted. For the generation of a TCR cell library expressing TCR with a mouse constant region an anti-mCB-specific antibody was used. For the generation of a TCR cell library expressing TCR with a human constant region an anti-CD3 antibody was used. The cell libraries are generated using transformed TCR-negative T cells in order to efficiently produce cellular reagents with uniform TCR expression specific for the AV or BV region of choice. Further, these cells have unlimited capacity for proliferation in vitro. One cell library is developed using murine TCR-negative cells (BW⁻¹⁻) and the second library is developed using TCR-negative human Jurkat T cells (FIG. 3).

The BW^{-/-} TCR cellular library was used for immunization. For this purpose, mice were immunized with chimeric TCR-expressing BW^{-/-} cells, which minimize the differences seen during immunization with whole cell immunogens. Despite this minimization of TCR immunogenicity to selected V regions, mice were still able to produce antibodies against other surface proteins expressed by BW^{-/-} cells. These included responses to allogeneic MHC molecules, dependent upon the immunized strains of mice. Furthermore, undefined surface proteins expressed by BW^{-/-} cells, associated with cellular transformation or viral transduction also served as immunogenic epitopes. Lastly, BW^{-/-} cells were found to bind mouse or rat Ig non-specifically.

In order to avoid that mabs were identified which do not react with TCR structures in primary screens the corresponding Jurkat^{-/-} library is used for screening (“cross-species screening”). Since Jurkat^{-/-} cells differ for MHC and other cell surface proteins from BW^{-/-} cells, they will not bind mabs specific for these molecules raised using BW^{-/-} cellular immunogens. Furthermore, Jurkat^{-/-} cells do not show non-specific binding of mouse or rat Ig.

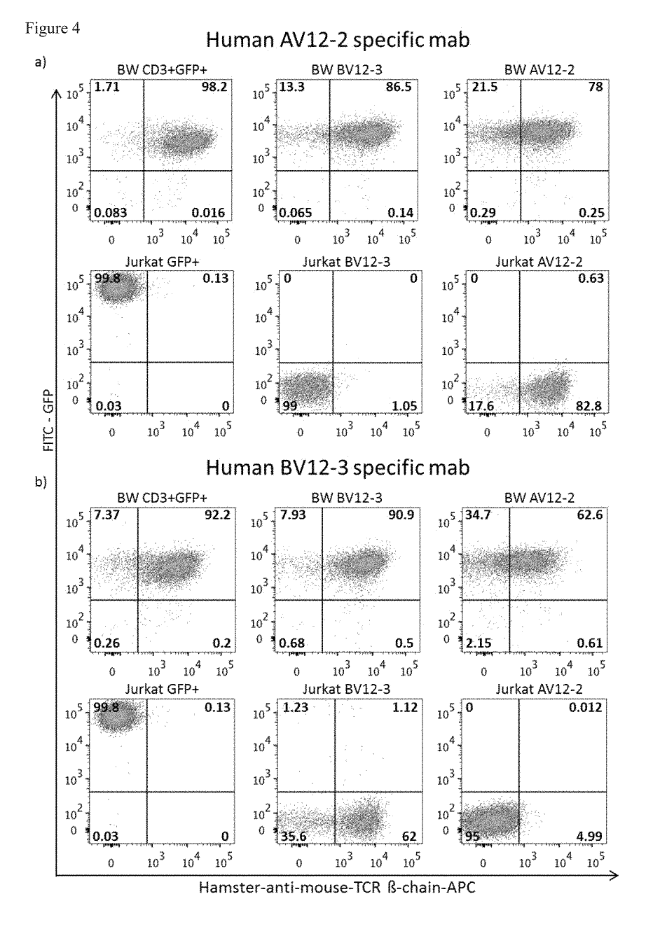

An example of cross-species screening is illustrated in FIG. 4, with two supernatants with putative specificity for human TCR AV12-2 and BV12-3 regions. As seen, all three BW^{-/-} cell lines bind both supernatants, irrespective of specific TCR expression, due to their property of non-specific Ig binding. In contrast, the Jurkat^{-/-} GFP control cells remain negative with both supernatants and each supernatant binds only to the TCR-transduced Jurkat^{-/-} cell line with the appropriate TCR.

### Mouse BW^{-/-} Cell Library Expressing Chimeric TCRs

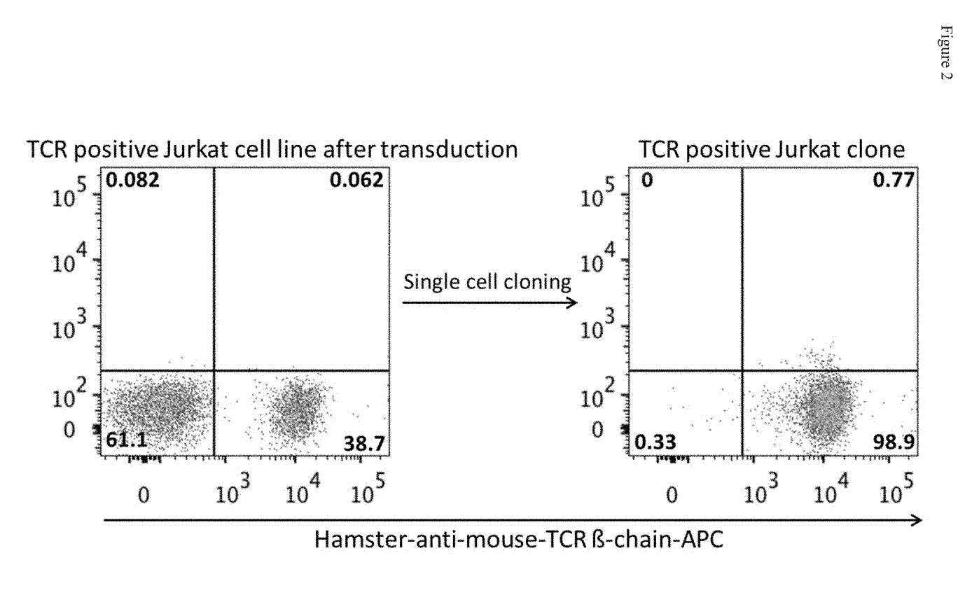

The mouse cell library is based on the BW^{-/-} cell line, which was derived from the parental BW5147 thymoma that arose spontaneously in an AKR mouse (Lee N E and Davis M M., J Immunol. 1988 Mar. 1; 140(5):1665-75; Letoumeur F., Malissen B., Eur J Immunol. 1989; 19(12):2269-2274). As described above, surface expression of a TCR heterodimer is dependent on association with the CD3 protein complex. Therefore, the BW^{-/-} cell line was stably transduced to co-express human CD3 with GFP (BW^{-/-}-CD3-GFP) (hereafter referred to simply as BW^{-/-}), enabling transduced cells to be easily identified. The presence of human CD3 allows these cells to express any human or mouse transgenic TCR at the cell surface after successful co-transduction with selected AV- and BV-encoding RVs. Surface expression can be monitored via binding of antibody specific for human CD3, or with antibody against the murine constant region, as shown in FIG. 2.

### Human Jurkat Cell Library Expressing Chimeric TCR

The second cell library is constructed using the human Jurkat TCL. The human Jurkat76^{-/-} cell line (hereafter Jurkat^{-/-}) is a variant of the original human TCL line that does not express human Vα and Vβ chains (Abraham R T, Weiss A., Nat Rev Immunol. 2004 April; 4(4):301-8). It has all remaining TCR-associated CD3 components necessary for transgenic TCR surface expression, after transduction with appropriate RVs of choice. As a negative control, Jurkat^{-/-} cells were made which express very high levels of GFP, but do not express TCR proteins.

Cross-Species Screening Using BW^{-/-} and Jurkat^{-/-} Cells.

BW-TCR transduced cells were used for immunization, however these cells could not be used for hybridoma screening since they bind mouse or rat Ig non-specifically as shown here for the anti-human AV12-2-specific hybridoma supernatant, as well as for the anti-human BV12-3-specific supernatant. Both hybridoma supernatants stain BW^{-/-} cells irrespective of their TCR expression (FIG. 4, first row in a and b). In contrast, the same supernatants stain Jurkat^{-/-} cells only when they express the specific AV or BV TCR chain (FIG. 4, second row a and b). As previously mentioned, TCR-transduced BW^{-/-} cells are stably transduced also with CD3-GFP in order to allow TCR expression, accounting for their intermediate level of GFP. To distinguish between non-specific and specific TCR binding on TCR-transduced Jurkat^{-/-} cells, a stably transduced GFP Jurkat^{-/-} cell clone was established and used as a control during hybridoma supernatant screening. As shown, Jurkat-GFP cells remain unlabeled when tested with supernatant containing either AV- or BV-specific mabs (FIG. 4, second row a and b).

### Lewis Rats Immunization

Lewis rats with were immunized with BW^{-/-} cells expressing hAV/hBV heterodimers containing mouse constant regions in combination or as single TCRs. The spleen cells of these rats were harvested and were fused to with myeloma cell line P3X63Ag8 and plated in twenty-four 96 well-plates. Two weeks later, an average of three hybridoma clones per well were observed throughout all plates, yielding approximately 6,900 hybridomas to be assessed.

### Primary Screening

For the first screening, supernatants from four 96-well-plates were pooled in one collecting plate for screening in flow cytometry. This reduced the sample number for the primary flow cytometry screen from twenty-four to six 96-well-plates.

In order to distinguish whether positive supernatants show mono-, cluster- or pan-TCR specificity during the primary screen, a pool of Jurkat cell clones, comprising a population expressing hAV3/hBV12-3 (45%), a population expressing Jurkat hAV8-2/hBV24 (45%) and one population of non TCR-transduced Jurkat-GFP cells (10%) were analyzed to identify non-TCR-specific mabs.

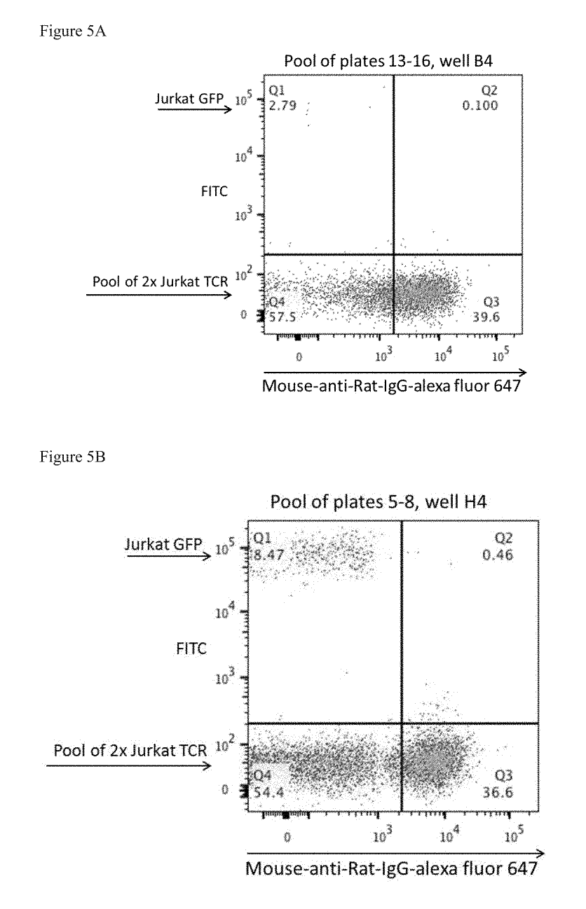

During the primary cross-species screen, for example one pooled supernatant of different antibody clones was found that bound around 40% of the screening pool (FIG. 5). This mab shows a TCR-associated binding pattern since Jurkat-GFP (TCR-negative) control cells did not show any shift in binding; Results of primary screening including clone 15B4 is shown in FIG. 5A. Results of primary screening including clone 5H4 is shown in FIG. 5B.

### Secondary Screening

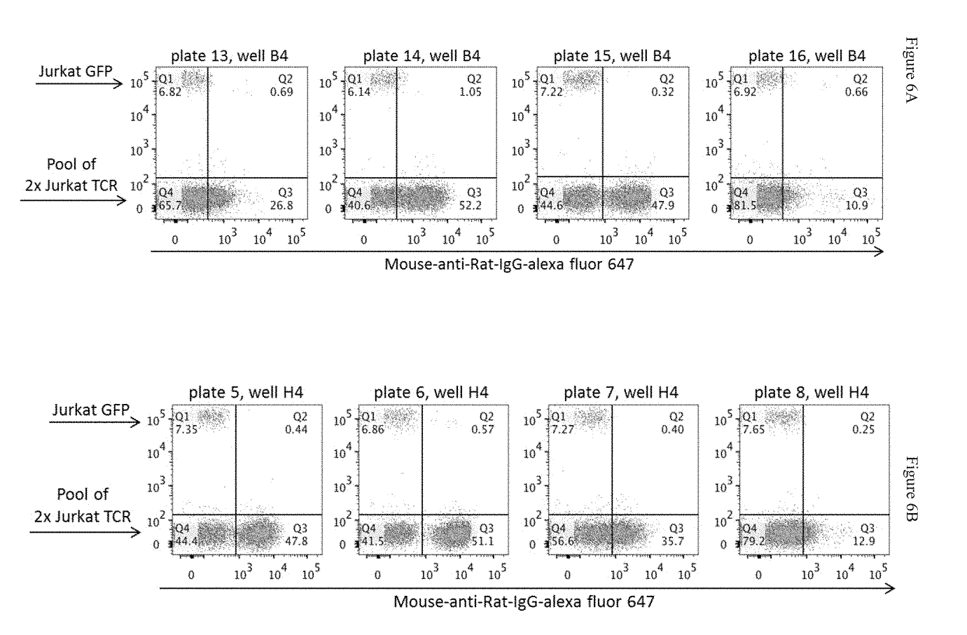

In order to identify the individual hybridoma responsible for the primary screening activity, the supernatants from location identified in the primary screen as having TCR associated binding pattern in the pooled supernatant of the primary screening were tested individually on the same pool of screening cells. One supernatant for example was found to reproduce the expected binding pattern, this hybridoma clone is indicated herein as 15B4 (FIG. 6A). Another supernatant for example was also found to reproduce the expected binding pattern, this hybridoma clone is indicated herein as 5H4 (FIG. 6B). These experiments establish that 15B4 as well as 5H4 recognize at least BV12-3. In the following, experiments are described which can be used to establish that 15B4 is a cluster TCR-specific mab.

### PBL Sorting

To differentiate whether a candidate antibody is mono-specific or cluster-specific (i.e. reacting with multiple BV chains that share amino acid homologies), PBL of a single human donor are stained with the candidate mab. The positive fraction of cells is sorted by flow cytometry and a full human TCR AV and BV PCR repertoire analysis is performed on the sorted cells.

From the PBL sorted PBL mRNA is extracted and cDNA prepared. The full human TCR AV and BV repertoire is analyzed by a standard PCR protocol (denaturation: 94° C. for 2 min; annealing: 35 cycles of: 94° C. for 30 sec, 55° C. for 30 sec, 72° C. for 1 min; extension: 72° C. for 1 min) using primer-pairs specific for each specific BV chain. Amplified bands are extracted and sequenced. Samples that show several amplicons of different TCR Vα or TCR Vβ chains will indicate that the candidate antibody is specific for a cluster of TCR Vα or TCR Vβ chains.

### Sequence Analysis of Sorted Cells

For antibodies binding to several TCR Vα or TCR Vβ chains, cells expressing BV chains for which the antibody is specific would be included in the sorted population. However, some contaminating cells might also be included in the sorted population, yielding a positive PCR amplicon due to the high sensitivity of the PCR method. In order to exclude amplicons due to contamination, all amplicon bands detected with BV-specific primers are sequenced. When the sequences are analyzed, the chromatopherograms as well as the density of the amplified bands were taken into account.

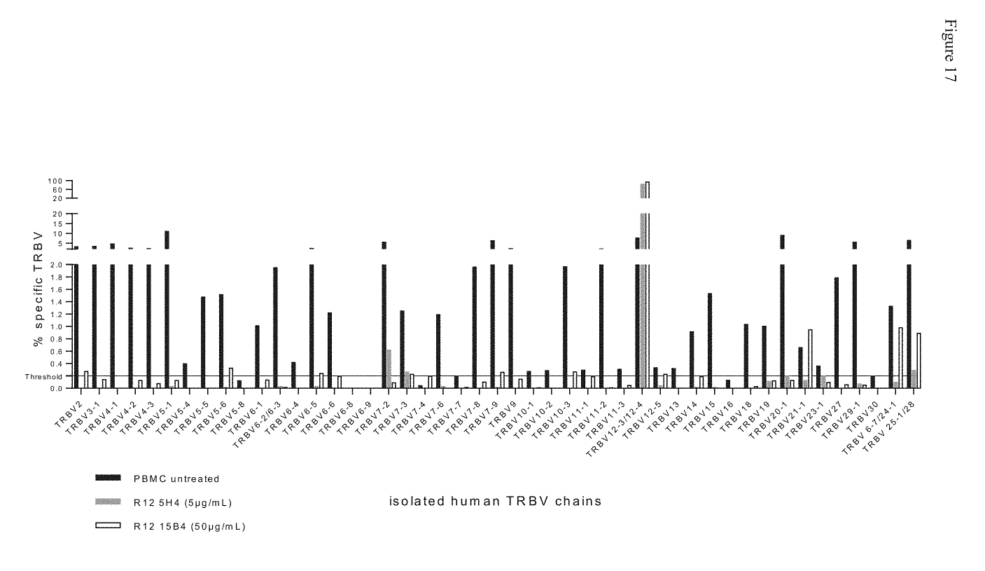

The previously described analysis of sequences obtained after sorting and Sanger sequencing of PCR amplicons generated from isolated lymphocyte population recognized by potential cluster antibodies indicated that these antibodies recognize a small cluster of BV chains. In order to more precisely determine the size and diversity of the clusters, the human PBMC were stained with R12 5H4 or R12 15B4 antibodies and positive populations sorted by FACS Aria sorting. The sorted cells are then used for preparation of library for next generation sequencing as published in Mamedov et al., (Front. Immunol. 2013; 4: 1-10). Briefly, the sorted lymphocytes were lysed and mRNA isolated using magnetic beads. This mRNA is then used for reverse transcription of cDNA coding for AV and BV TCR chains using smart cDNA synthesis technology. The AV and BV cDNA from each sample were then amplified in first PCR using universal primers specific for AV and BV chains. During this procedure the samples were labeled with unique barcodes on their 3′ and 5′ ends so that multi-sample NGS library can be sequenced and subsequently data analyzed and assigned for each specific sample. The illumina adaptor sequences needed for sequencing on MiSeq instrument by using illumina MiSeq Reagent Kits v2 chemistry were added during second PCR. The expected 600 base pair DNA band obtained after amplification of AV and BV chains containing respective adaptor sequences were extracted after classical DNA gel electrophoresis and NGS library for sequencing on MiSeq instrument prepared by illumina protocol. Upon sequencing, bioinformatic data analysis was performed and data presented in FIG. 17. The sequencing of whole PBMC repertoire by the same procedure was used as internal control.

### ADCC Reporter Bioassay (Promega)

ADCC is measured using ADCC Reporter Bioassay (Promega) according to the manufacturer's protocol. In short, the activation of gene transcription through the NFAT (nuclear factor of activated T-cells) pathway in the effector cell is monitored by the luciferase activity in the effector cell which is quantified with luminescence readout. The ADCC Reporter Bioassay uses engineered Jurkat cells stably expressing the FcγRIIIa-receptor, V158 (high affinity) variant, and an NFAT response element driving expression of firefly luciferasease effector cells. The biological activity of the antibody in ADCC MOA is quantified through the luciferase produced as a result of NFAT pathway activation;

### ADDC Assay: Human Natural Killer Target Cell Visualization Assay (TVA™)

A preferred assay for the measuring ADCC is the Human Natural Killer Target Cell Visualization Assay (TVA™; C.T.L., Cell Technology Limited). The capacity of the identified antibodies to induce ADCC (antibody-dependent cellular cytotoxicity) was assessed using this non-radioactive target cell visualization assay.

The TVA™ utilizes direct imaging of fluorescence-labeled target cells and was performed according to the instructions of the manufacturer. Shortly, 5E+03 labeled Jurkat cells expressing either the TCR variable beta chain 12-3 (recognized by the antibody candidates) or BV3-1 (not recognized by the antibodies) were co-incubated with IL-2 stimulated NK cells, isolated from the blood of healthy donors. The cells were co-cultured for 4-6 hours at varying target-to-effector ratios in combination with the antibody candidates. Following NK-mediated lysis, target cells lose their fluorescent signal. The direct visualization of remaining viable target cells at the end of an assay period determines the percentage of cytotoxicity for each T:E ratio. % cytotoxicity was assessed using following formula: % killing sample=100−((100×sample)/target cells only).

### In Vivo Depletion of T Cells Expressing BV12-3-Related TCRs in a Humanized TCR Mouse Model

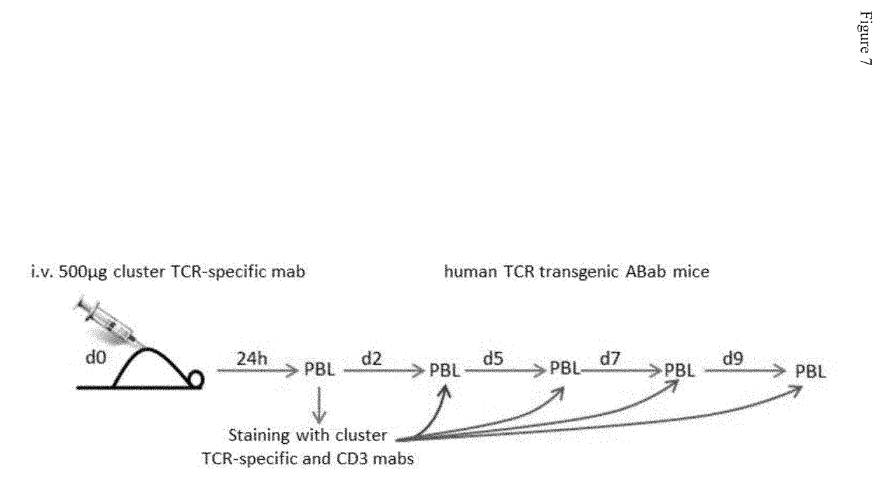

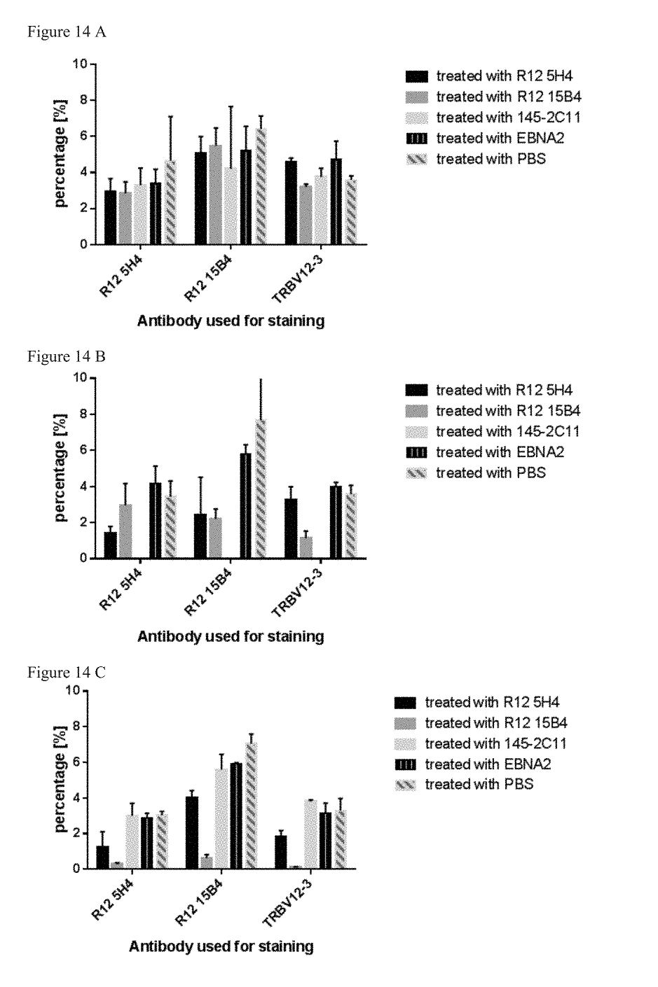

ABab mice expressing human AV and BV (hAV/hBV) TCR chains were treated with the antibodies 5H4 and 15B4 (500 μg/mouse). Each antibody was applicated to three mice/group. Also included in the experimental setting were three control groups with 3 animals each. One group was treated with antibody 145-2C11 (anti-mouse CD3) as a positive control, since this antibody depletes all T-cells and induces a severe cytokine storm. One group was treated with an isotype control antibody (anti-EBNA2), which should not lead to any depletion since its recognized epitope is not present in mice. The last control group was left untreated (PBS) serving as a naive control. The PBL acquired by tail bleeds were analyzed before treatment (dO) and after treatment on day 4 and 21 (alternatively also bleeds for example at days 2, 5, 7 and 9 can be used) by staining with anti-CD3-PE and candidate anti-TCR variable chain mab in combination with mouse anti-rat-IgG secondary antibody (see also FIGS. 7 and 14).

The population of CD3+ T cells identified by the anti-TCR variable chain antibodies should remain stable in both groups treated with EBNA2 or the naive group throughout the whole experimental time course. In contrast, T cells expressing the respective TCR variable chains disappeared in the groups treated with the anti-TCR variable chain antibodies within 21 days (FIG. 14 C; also shown 4 days: FIG. 14 B; without treatment: FIG. 14 A). The remaining T cells, represented as a CD3+ and TCR variable chain antibody negative population should remained stable at the same level during the experiment. In the positive control group, all CD3+ T cells should disappear within 4 days.

In order to examine in detail the in vivo effects of the candidate TCR V chain mab, the size of the T cell population to the candidate antibody is assessed in comparison with the size of the T cell population detected using a commercial mab that is mono-specific for a TCR Vα chain or a TCR Vβ chain. This comparison is made before and after in vivo depletion studies. The experiments include mab staining of PBL of human TCR transgenic (ABab) mice and wild type C57BL/6 mice on day 0 and 48 hrs later.

To determine which BV chains are targeted by the candidate mab, a complete TCR BV repertoire analysis is prepared by using individual BV chain-specific primers. The PBL from human TCR transgenic mice (ABab) depleted with the candidate antibody (500 μg) are collected and the complete BV repertoire is determined using individual BV-specific primers.

### Cytokine Measurement During In Vivo Depletion

Many mab that target T cell structures, including those specific for CD3 (145-2C11 control antibody in this experiment) or CD28 receptors, induce rapid systemic release of many cytokines from T cells that are involved in immune responses. Mabs recognizing a structural region of the TCR that is not directly involved in recognition of the antigen-MHC complex and is not involved in TCR signaling should be safe and only pathogenic T cells should be depleted, without evoking a toxic cytokine storm.

In order to measure cytokine release in mice during in vivo T cell depletion, serum concentrations of IL-2, IL-4, IL-6, IL-10, IFN-gamma and TNF-alpha were measured 0, 2, 6, 12, and 24 hours after mab application using a bead based flow cytometry assay (alternatively ELISA could be used). The same groups as descripted in section “In vivo depletion of T cells expressing BV12-3-related TCRs in a humanized TCR mouse model”) were analyzed: Each mouse receives 500 μg of purified mab. Besides the antibodies 5H4 and 15B4, 3 controls were included: The first control group is treated with isotype control mab of the rat IgG2a isotype. This mab recognizes the Epstein-Barr-Virus antigen EBNA2. This mab should not show any effect in treated mice. The second control is a hamster anti-mouse CD3 mab (IgG1 anti-mouse CD3 zeta, clone 145-2C11), which is known to induce cytokine storm in treated animals (Hirsch, 1988; Penaranda, 2011). The last control group was left untreated serving as a naïve control.

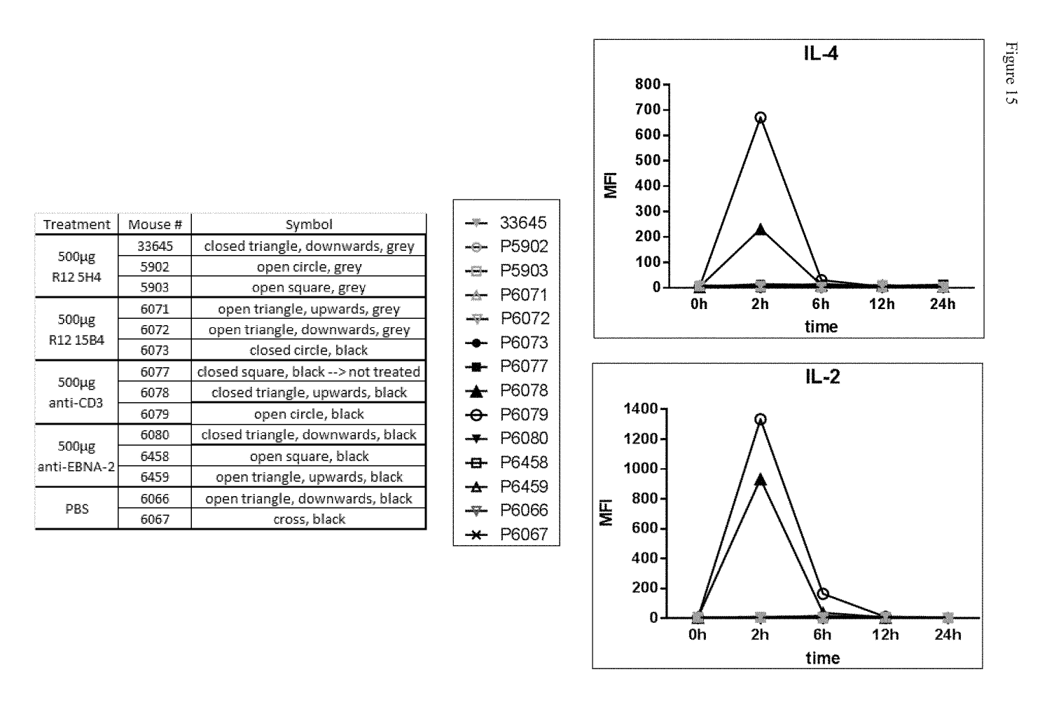

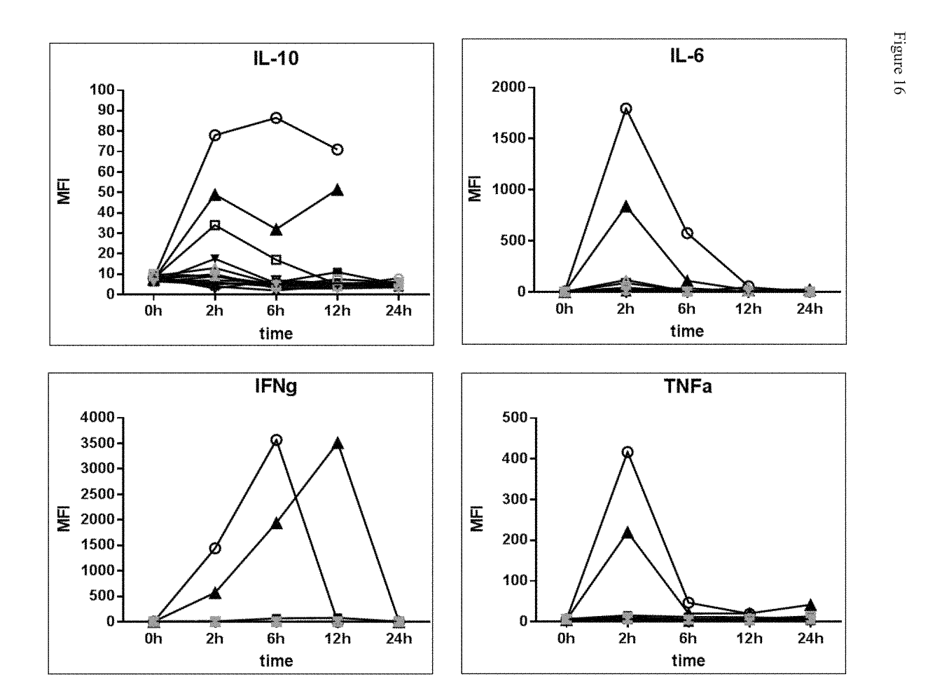

Treatment with anti-EBNA2 mab shows no effect on cytokine production. In contrast treatment with anti-CD3 mab induced a cytokine storm, including the release of IL-2, IL-4, IL-6 and IFN-γ about 2 hrs after application. However, the candidate mabs did not increase cytokine levels in serum of the treated animals. Increased levels of IL-10 and TNF-α may be detected at later time points (e.g. 12 hrs) indicating that an inflammatory response may be occurring in vivo, perhaps involving macrophage activation through phagocytosis of targeted T cells due to the time delay. However, also these effects could not be detected with the antibodies 5H4 and 15B4 (FIGS. 15 and 16).

### Assembled Library

A library is constructed comprising the following TCR constructs:

TABLE 8
constructed TCR library

| TCR | C | SEQ ID | V | SEQ ID | | SEQ ID |
|---|---|---|---|---|---|---|
| construct | segment | NO: | segment | No: | linker sequence | No: |
| TCRA1 | AC | 6 | AVseg1 | 8 | linker specific for A | 192 |
| TCRA2 | AC | 6 | AVseg2 | 9 | linker specific for A | 192 |
| TCRA3 | AC | 6 | AVseg3 | 10 | linker specific for A | 192 |
| TCRA4 | AC | 6 | AVseg4 | 11 | linker specific for A | 192 |
| TCRA5 | AC | 6 | AVseg5 | 12 | linker specific for A | 192 |
| TCRA6 | AC | 6 | AVseg6 | 13 | linker specific for A | 192 |
| TCRA7 | AC | 6 | AVseg7 | 14 | linker specific for A | 192 |
| TCRA8 | AC | 6 | AVseg8 | 15 | linker specific for A | 192 |
| TCRA9 | AC | 6 | AVseg9 | 16 | linker specific for A | 192 |
| TCRA10 | AC | 6 | AVseg10 | 17 | linker specific for A | 192 |
| TCRA11 | AC | 6 | AVseg11 | 18 | linker specific for A | 192 |
| TCRA12 | AC | 6 | AVseg12 | 19 | linker specific for A | 192 |
| TCRA13 | AC | 6 | AVseg13 | 20 | linker specific for A | 192 |
| TCRA14 | AC | 6 | AVseg14 | 21 | linker specific for A | 192 |
| TCRA15 | AC | 6 | AVseg15 | 22 | linker specific for A | 192 |
| TCRA16 | AC | 6 | AVseg16 | 23 | linker specific for A | 192 |
| TCRA17 | AC | 6 | AVseg17 | 24 | linker specific for A | 192 |
| TCRA18 | AC | 6 | AVseg18 | 25 | linker specific for A | 192 |
| TCRA19 | AC | 6 | AVseg19 | 26 | linker specific for A | 192 |
| TCRA20 | AC | 6 | AVseg20 | 27 | linker specific for A | 192 |
| TCRA21 | AC | 6 | AVseg21 | 28 | linker specific for A | 192 |
| TCRA22 | AC | 6 | AVseg22 | 29 | linker specific for A | 192 |
| TCRA23 | AC | 6 | AVseg23 | 30 | linker specific for A | 192 |
| TCRA24 | AC | 6 | AVseg24 | 31 | linker specific for A | 192 |
| TCRA25 | AC | 6 | AVseg25 | 32 | linker specific for A | 192 |
| TCRA26 | AC | 6 | AVseg26 | 33 | linker specific for A | 192 |
| TCRA27 | AC | 6 | AVseg27 | 34 | linker specific for A | 192 |
| TCRA28 | AC | 6 | AVseg28 | 35 | linker specific for A | 192 |
| TCRA29 | AC | 6 | AVseg29 | 36 | linker specific for A | 192 |
| TCRA30 | AC | 6 | AVseg30 | 37 | linker specific for A | 192 |
| TCRA31 | AC | 6 | AVseg31 | 38 | linker specific for A | 192 |
| TCRA32 | AC | 6 | AVseg32 | 39 | linker specific for A | 192 |
| TCRA33 | AC | 6 | AVseg33 | 40 | linker specific for A | 192 |
| TCRA34 | AC | 6 | AVseg34 | 41 | linker specific for A | 192 |
| TCRA35 | AC | 6 | AVseg35 | 42 | linker specific for A | 192 |
| TCRA36 | AC | 6 | AVseg36 | 43 | linker specific for A | 192 |
| TCRA37 | AC | 6 | AVseg37 | 44 | linker specific for A | 192 |
| TCRA38 | AC | 6 | AVseg38 | 45 | linker specific for A | 192 |
| TCRA39 | AC | 6 | AVseg39 | 46 | linker specific for A | 192 |
| TCRA40 | AC | 6 | AVseg40 | 47 | linker specific for A | 192 |
| TCRA41 | AC | 6 | AVseg41 | 48 | linker specific for A | 192 |
| TCRA42 | AC | 6 | AVseg42 | 49 | linker specific for A | 192 |
| TCRA43 | AC | 6 | AVseg43 | 50 | linker specific for A | 192 |
| TCRA44 | AC | 6 | AVseg44 | 51 | linker specific for A | 192 |
| TCRA45 | AC | 6 | AVseg45 | 52 | linker specific for A | 192 |
| TCRB1 | BC1 | 7 | BVseg1 | 53 | linker specific for B | 193 |
| TCRB2 | BC1 | 7 | BVseg2 | 54 | linker specific for B | 193 |
| TCRB3 | BC1 | 7 | BVseg3 | 55 | linker specific for B | 193 |
| TCRB4 | BC1 | 7 | BVseg4 | 56 | linker specific for B | 193 |
| TCRB5 | BC1 | 7 | BVseg5 | 57 | linker specific for B | 193 |
| TCRB6 | BC1 | 7 | BVseg6 | 58 | linker specific for B | 193 |
| TCRB7 | BC1 | 7 | BVseg7 | 59 | linker specific for B | 193 |
| TCRB8 | BC1 | 7 | BVseg8 | 60 | linker specific for B | 193 |
| TCRB9 | BC1 | 7 | BVseg9 | 61 | linker specific for B | 193 |
| TCRB10 | BC1 | 7 | BVseg10 | 62 | linker specific for B | 193 |
| TCRB11 | BC1 | 7 | BVseg11 | 63 | linker specific for B | 193 |
| TCRB12 | BC1 | 7 | BVseg12 | 64 | linker specific for B | 193 |
| TCRB13 | BC1 | 7 | BVseg13 | 65 | linker specific for B | 193 |
| TCRB14 | BC1 | 7 | BVseg14 | 66 | linker specific for B | 193 |
| TCRB15 | BC1 | 7 | BVseg15 | 67 | linker specific for B | 193 |
| TCRB16 | BC1 | 7 | BVseg16 | 68 | linker specific for B | 193 |
| TCRB17 | BC1 | 7 | BVseg17 | 69 | linker specific for B | 193 |
| TCRB18 | BC1 | 7 | BVseg18 | 70 | linker specific for B | 193 |
| TCRB19 | BC1 | 7 | BVseg19 | 71 | linker specific for B | 193 |
| TCRB20 | BC1 | 7 | BVseg20 | 72 | linker specific for B | 193 |
| TCRB21 | BC1 | 7 | BVseg21 | 73 | linker specific for B | 193 |
| TCRB22 | BC1 | 7 | BVseg22 | 74 | linker specific for B | 193 |
| TCRB23 | BC1 | 7 | BVseg23 | 75 | linker specific for B | 193 |
| TCRB24 | BC1 | 7 | BVseg24 | 76 | linker specific for B | 193 |
| TCRB25 | BC1 | 7 | BVseg25 | 77 | linker specific for B | 193 |
| TCRB26 | BC1 | 7 | BVseg26 | 78 | linker specific for B | 193 |
| TCRB27 | BC1 | 7 | BVseg27 | 79 | linker specific for B | 193 |
| TCRB28 | BC1 | 7 | BVseg28 | 80 | linker specific for B | 193 |
| TCRB29 | BC1 | 7 | BVseg29 | 81 | linker specific for B | 193 |
| TCRB30 | BC1 | 7 | BVseg30 | 82 | linker specific for B | 193 |
| TCRB31 | BC1 | 7 | BVseg31 | 83 | linker specific for B | 193 |
| TCRB32 | BC1 | 7 | BVseg32 | 84 | linker specific for B | 193 |
| TCRB33 | BC1 | 7 | BVseg33 | 85 | linker specific for B | 193 |
| TCRB34 | BC1 | 7 | BVseg34 | 86 | linker specific for B | 193 |
| TCRB35 | BC1 | 7 | BVseg35 | 87 | linker specific for B | 193 |
| TCRB36 | BC1 | 7 | BVseg36 | 88 | linker specific for B | 193 |
| TCRB37 | BC1 | 7 | BVseg37 | 89 | linker specific for B | 193 |
| TCRB38 | BC1 | 7 | BVseg38 | 90 | linker specific for B | 193 |
| TCRB39 | BC1 | 7 | BVseg39 | 91 | linker specific for B | 193 |
| TCRB40 | BC1 | 7 | BVseg40 | 92 | linker specific for B | 193 |
| TCRB41 | BC1 | 7 | BVseg41 | 93 | linker specific for B | 193 |
| TCRB42 | BC1 | 7 | BVseg42 | 94 | linker specific for B | 193 |
| TCRB43 | BC1 | 7 | BVseg43 | 95 | linker specific for B | 193 |
| TCRB44 | BC1 | 7 | BVseg44 | 96 | linker specific for B | 193 |
| TCRB45 | BC1 | 7 | BVseg45 | 97 | linker specific for B | 193 |
| TCRB46 | BC1 | 7 | BVseg46 | 98 | linker specific for B | 193 |
| TCRB47 | BC1 | 7 | BVseg47 | 99 | linker specific for B | 193 |

An additional library is constructed which comprises TCRA1 to TCRA45 constructs which correspond to the TCRA1 to TCRA45 constructs identified Table 8, except that they contain a human constant a AC segment (SEQ ID No: 1) instead of mouse constant AC segment (SEQ ID No: 6) and which further comprises TCRB1 to TCRB47 construct which correspond to the TCRB1 to TCRB47 construct identified in Table 8, except that they contain a human constant BC segment (SEQ ID No:4) instead of mouse constant BC segment (SEQ ID No: 7).

The TCR constructs TCRA1 to TCRA45 and the TCRB1 to TCRB47 have been integrated into the ivtRNA backbone vector SEQ ID No: 196.

The TCR constructs TCRA1 to TCRA45 and the TCRB1 to TCRB47 have been integrated into the retroviral backbone vector SEQ ID No: 200.

The constructs TCRA1 and TCRB12 have been integrated into the ivtRNA backbone AC-P2A-BC (SEQ ID No: 199).

The constructs TCRA11 and TCRB12 have been integrated into the retroviral backbone vector AC-P2A-BC (SEQ ID No: 203).

### Reengineering of an Isolated TCR

### Functional Analysis of T Cell Clone T1.8-3-200

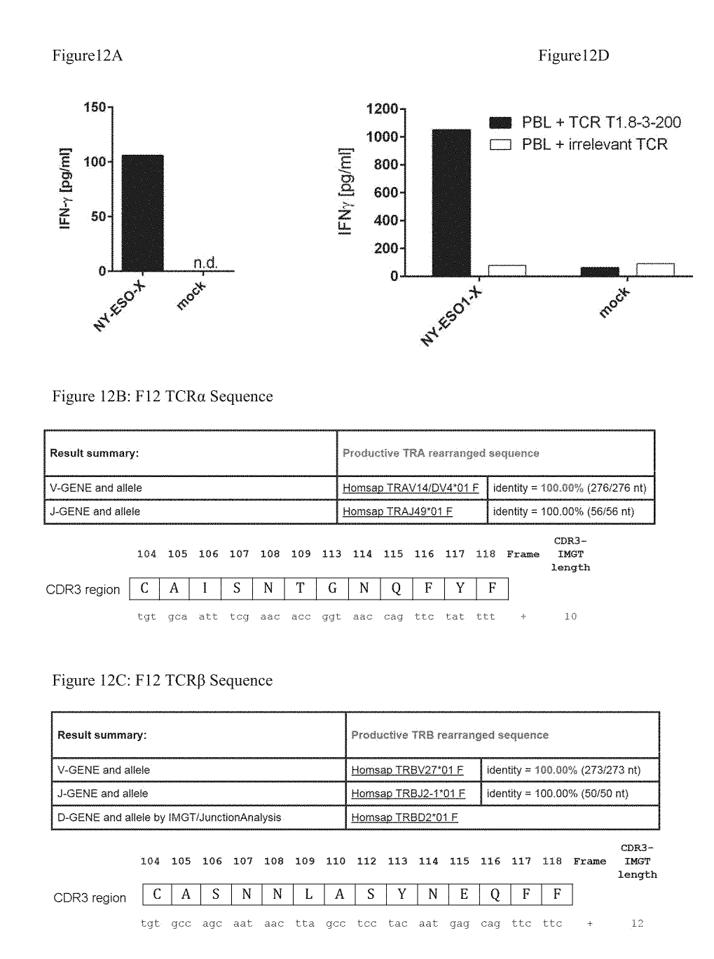

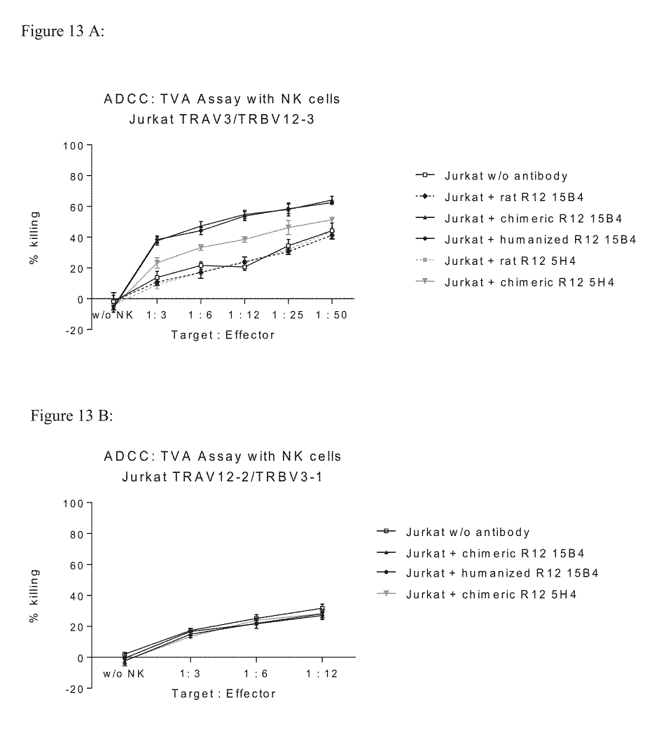

Co-culture of T cell clone T1.8-3-200 with HLA-matched NY-ESO1-X-(human NY-ESO1 antigen fused to a signal peptide)-loaded APC demonstrated the specificity and function of clone T1.8-3-200 (n.d., not detectable; FIG. 12A).

### TCR Analysis of Original T Cell Clone T1.8-3-200

Rearranged TCR DNA sequences ofT cell clone T1.8-3-200 were amplified by 5′RACE PCR. For this, whole RNA was isolated from T1.8-3-200 (recognizing human NY-ESO1 antigen fused to a signal peptide; NY-ESO1-X) T cells and reverse transcribed to complementary DNA (cDNA). The rearranged TCRα and β sequences were subsequently amplified by 5′RACE amplification. Using TOPO cloning, the amplified DNA fragments were cloned into an adequate recipient vector to allow the isolation of individual TCR DNA sequences after bacterial transformation.

### DNA Sequencing

TCR sequence inserts from vectors that were isolated from single bacterial colonies were analyzed by DNA nucleotide sequencing.

T1.8-3-200 TCRα sequencing result (SEQ ID No:
210):
atgtcactactagcctgctgaaggtggtcacagcttcactgtggctagga
cctggcattgcccagaagataactcaaacccaaccaggaatgttcgtgca
ggaaaaggaggctgtgactctggactgcacatatgacaccagtgatccaa
gttatggtctattctggtacaagcagcccagcagtggggaaatgattttt
cttatttatcaggggtcttatgaccagcaaaatgcaacagaaggtcgcta
ctcattgaatttccagaaggcaagaaaatccgccaaccttgtcatctccg
cttcacaactgggggactcagcaatgtacttctgtgcaatttcgaacacc
ggtaaccagttctattttgggacagggacaagtttgacggtcattccaaa
tatccagaaccctgaccctgccgtgtaccagctgagagactctaaatcca
gtgacaagtctgtctgcctattcaccgattttgattctcaaacaaatgtg
tcacaaagtaaggattctgatgtgtatatcacagacaaaactgtgctaga
catagtcagg
T1.8-3-200 TCRβ sequencing result (SEQ ID No:
211):
atgggcccccagctccttggctatgtggtcctttgccttctaggagcagg
ccccctggaagcccaagtgacccagaacccaagatacctcatcacagtga
ctggaaagaagttaacagtgacttgttctcagaatatgaaccatgagtat
atgtcctggtatcgacaagacccagggctgggcttaaggcagatctacta
ttcaatgaatgttgaggtgactgataagggagatgttcctgaagggtaca
aagtctctcgaaaagagaagaggaatttccccctgatcctggagtcgccc
agccccaaccagacctctctgtacttctgtgccagcaataacttagcctc
ctacaatgagcagttcttcgggccagggacacggctcaccgtgctagagg
acctgaaaaacgtgttcccacccgaggtcgctgtgtttgagccatcagaa
gcagagatctcccacacccaaaaggccacactggtgtgcctggccacagg
cttctaccccgaccacgtggagctgagctggtgggtgaatgggaaggagg
tgcacagtggggtcagcacagacccgcagcccctcaagagcagcgctt

### IMGT Sequence Analysis

The TCR specificity-defining parameters (rearranged TCR V-(D)-Jα/β segments, sequence of CDR3 region and employed Cα/β region) were analyzed from the retrieved DNA sequences using the IMGT/V-QUEST search platform (www.imgt.org); The results for the TCR α and the TCRβ chain are shown in FIG. 12B and FIG. 12C respectively.

Identified T1.8-3-200 TCRα CDR3 sequences:
DNA Sequence:
(SEQ ID No: 243)
gcaatttcgaacaccggtaaccagttctat
Protein Sequence:
(SEQ ID No: 245)
AISNTGNQFY
Identified T1.8-3-200 TCRβ CDR3 Sequences:
DNA Sequence:
(SEQ ID No: 244)
gccagcaataacttagcctcctacaatgagcagttc
Protein Sequence:
(SEQ ID No: 246)
ASNNLASYNEQ

### Reconstruction in TCR Vector Library

Appropriate vectors from the pGEM-based TCR vector library with human constant regions were used to reconstruct the T1.8-3-200 TCRα/β chains by exchanging the generic CDR3 linker with annealed DNA oligonucleotides coding for the respective T1.8-3-200 TCRα/β CDR3+J region (restriction sites: FspI x BspEI (TCRα chain; AV14 vector); FspI x BstEII (TCRβ chain; BV27 vector)).

T1.8-3-200 TCRα oligonucleotide Sense 5′→3′
(SEQ ID No: 239):
GCAATCAGCAACACCGGCAACCAGTTCTACTTCGGCACCGGCACCAGCCT
GACCGTGATCCCCAACATCCAGAAT
T1.8-3-200 TCRα oligonucleotide Antisense 5′→3′
(SEQ ID No: 240):
CCGGATTCTGGATGTTGGGGATCACGGTCAGGCTGGTGCCGGTGCCGAAG
TAGAACTGGTTGCCGGTGTTGCTGATTGC
T1.8-3-200 TCRβ oligonucleotide Sense 5′→3′
(SEQ ID No: 241):
GCAAGCAACAACCTGGCCAGCTACAACGAGCAGTTCTTCGGCCCTGGCAC
CCGGCTGACCGTGCTGGAAGATCTGAAGAACGTGTTCCCCCCAGAG
T1.8-3-200 TCRβ oligonucleotide Antisense 5′→3′
(SEQ ID No: 242):
GTCACCTCTGGGGGGAACACGTTCTTCAGATCTTCCAGCACGGTCAG
CCGGGTGCCAGGGCCGAAGAACTGCTCGTTGTAGCTGGCCAGGTTGT
TGCTTGC

The sequences of the reconstructed T1.8-3-200 TCRα plasmids are set out in SEQ ID No: 208 (pMP71 based retroviral vector) and SEQ ID No: 251(ivtRNA vector). The sequences of reconstructed T1.8-3-200 TCRβ plasmids are set out in SEQ ID No: 209 (retroviral vector) and SEQ ID No: 252 (pGEM based ivtRNA vector). The nucleotide sequence of the reconstructed TCR α chain is set out in SEQ ID No: 247, the corresponding amino acid sequence is set out in SEQ ID No: 249. The nucleotide sequence of the reconstructed TCR β chain is set out in SEQ ID No: 248, the corresponding amino acid sequence is set out in SEQ ID No: 250.

### Transgenic Function Analysis of TCR T1.8-3-200

RNA coding for T1.8-3-200 TCRα/β chains was produced from the generated pAV/BV-T1.8-3-200-ivtRNA vector constructs and used for transfection of peripheral blood lymphocyts (PBL). Co-culture of the T1.8-3-200 TCR-transfected PBL with HLA-matched NY-ESO1-X-loaded APC demonstrated the restoration of the previously defined T1.8-3-200 TCR specificity and function in the recipient T cells (FIG. 12D).

The application further comprises the following embodiments:

The application further comprises the following embodiments:

### Embodiment 1

Antibody or binding fragment thereof that binds to a fraction of T cell receptor variable alpha (TCR Vα) chains comprising at least two different TCR Vα chains but less than all TCR Vα chains or that binds to a fraction of T cell receptor variable beta (TCR Vβ) chains comprising at least two different TCR Vβ chains but less than all TCR Vβ chains.

### Embodiment 2

Antibody or binding fragment thereof according to embodiment 1 wherein the fraction of TCR Vα chains comprises least two different TCR Vα chains that belong to at least two different TCR Vα chain subfamilies or wherein the fraction TCR Vβ chains comprises least to two different TCR Vβ chains that belong to at least two different TCR Vβ chain subfamilies.

### Embodiment 3

Antibody or binding fragment thereof according to embodiment 1 or 2 that binds to a fraction of TCR Vα chains that comprises at least 3, at least 4, at least 5, at least 6 different TCR Vα chains or to a fraction of TCR Vβ chains comprising at least 3, at least 4, at least 5, at least 6 different TCR Vβ chains.

### Embodiment 4

Antibody or binding fragment thereof according to any of embodiment 1, 2, or 3 that binds to a fraction of TCR Vα chains that comprises at least 3, at least 4, at least 5, at least 6 different TCR Vα chains or to a fraction of TCR Vβ chains comprising at least 3, at least 4, at least 5, at least 6 different TCR Vβ chains and wherein said at least at least 3, at least 4, at least 5, at least 6 different TCR Vα or VB chains belong to at least 3, at least 4, at least 5, at least 6 different TCR Vα or VB chain subfamilies.

### Embodiment 5

Antibody or binding fragment thereof according to any of the preceding embodiments wherein the fraction of TCR Vα chains comprises at least 5%, at least 10%, at least 25%, at least 30%, at least 40%, at least 50% of the total amount TCR Vα chains expressed on T cells or wherein the fraction of TCR Vβ chains comprises at least 5%, at least 10%, at least 25%, at least 30%, at least 40%, at least 50% of the total amount TCR Vβ chains expressed on T cells.

### Embodiment 6

Antibody or binding fragment thereof according to any of the preceding embodiments wherein the antibody or binding fragment thereof binds to a fraction of TCR Vβ chains.

### Embodiment 7

Antibody or binding fragment thereof according to any of the preceding embodiments, wherein the fraction comprises at least two different TCR Vβ chains that belong to at least two different subfamilies selected from the group comprising BV4, BV6, BV7, BV12, BV14, BV18, BV20, BV21, BV23, BV24 and BV29.

### Embodiment 8

Antibody or binding fragment thereof according to any of the preceding embodiments, wherein the fraction comprises at least 2, preferably at least 3, more preferably at least 4 different TCR Vβ chains selected from the group comprising BV6-4, BV6-5, BV6-6, BV12-3, BV12-5, BV14-1, BV18-1, BV20-1, BV21-1, BV24-1 and BV29-1.

### Embodiment 9

Antibody or binding fragment thereof according to any of the preceding embodiments, wherein the fraction comprises at least two, preferably at least 3, more preferably at least 4 different TCR Vβ chains that belong to at least two subfamilies selected from the group comprising BV6, BV12, BV14, BV18, BV20, BV21, BV24 and BV29.

### Embodiment 10

Antibody or binding fragment thereof according to any of the preceding embodiments, wherein the fraction comprises at least two TCR Vβ chains that belong to least two different subfamilies selected from the group comprising BV6, BV12, BV14, BV18, BV20, BV24, BV29.

### Embodiment 11

Antibody or binding fragment thereof according to any of the preceding embodiments, wherein the fraction comprises at least two TCR Vβ chains that belong to least two different subfamilies selected from the group comprising BV12, BV14, BV18, BV24 and BV29.

### Embodiment 12

Antibody or binding fragment thereof according to any of the preceding embodiments, wherein the fraction comprises at least two TCR Vβ chains that belong to least two different subfamilies selected from the group comprising BV12, BV14, BV24 and BV29.

### Embodiment 13

Antibody or binding fragment thereof according to any of the preceding embodiments, wherein the antibody or binding fragment thereof binds to at least two different TCR Vβ chains selected from the group comprising BV12-3, BV14-1 and BV24-1.

### Embodiment 14

Antibody or binding fragment thereof according to any of the preceding embodiments, wherein the antibody or binding fragment thereof does not induce the release of proinflammartory cytokines in the form of a cytokine storm.

### Embodiment 15

Antibody or binding fragment thereof according to the preceding embodiments wherein the antibody or binding fragment thereof is capable of depleting a subpopulation of T cells expressing a fraction of TCR Vα chains comprising at least two different TCR Vα chains or is capable of depleting a subpopulation of T cells expressing a fraction of TCR Vβ chains comprising at least two different TCR Vβ chains.

### Embodiment 16

Antibody or binding fragment thereof according to embodiment 2 wherein the subpopulation of depleted T cells expresses a fraction of TCR Vα chains comprising at least two different TCR Vα chains to which the antibody is binding or expresses a fraction of TCR Vβ chains comprising at least two different TCR Vβ chains to which the antibody is binding.

### Embodiment 17

Antibody or binding fragment thereof that is capable of depleting a subpopulation of T cell expressing a fraction of TCR Vα chains comprising at least two different TCR Vα chains or that is capable of depleting a subpopulation of T cell expressing a fraction of TCR V β chains comprising two different TCR Vβ chains.

### Embodiment 18

Antibody or binding fragment thereof according to any of the preceding embodiments comprising a light chain variable region and/or a heavy chain variable region, wherein the light chain variable region comprises at least a CDR1 set forth in SEQ ID No: 212 or sequences at least 80% identical thereto, a CDR2 set forth in SEQ ID No: 213 or sequences at least 80% identical thereto, and/or a CDR3 set forth in SEQ ID No: 214 or sequences at least 80% identical thereto; and/or wherein the heavy chain variable region comprises at least a CDR1 set forth in SEQ ID No: 215 or sequences at least 80% identical thereto, a CDR2 set forth in SEQ ID No: 216 or sequences at least 80% identical thereto, and/or a CDR3 set forth in SEQ ID No: 217 or sequences at least 80% identical thereto.

### Embodiment 19

Antibody or binding fragment thereof according to embodiment 18 comprising a light chain variable region comprising SEQ ID No: 218 or sequences at least 80% identical thereto and/or a heavy chain variable region comprising SEQ ID No: 219 or sequences at least 80% identical thereto.

### Embodiment 20

Antibody or binding fragment thereof according to embodiment 18 or 19 wherein the antibody comprises a light chain variable region and/or a heavy chain variable region, wherein the light chain variable region comprises at least a CDR1 set forth in SEQ ID No: 212, a CDR2 set forth in SEQ ID No: 213, and/or a CDR3 set forth in SEQ ID No: 214; and/or wherein the heavy chain variable region comprises at least a CDR1 set forth in SEQ ID No: 215, a CDR2 set forth in SEQ ID No: 216, and/or a CDR3 set forth in SEQ ID No: 217.

### Embodiment 21

Antibody or binding fragment thereof according to embodiment 20 wherein the antibody or binding fragment thereof comprises a light chain variable region comprising SEQ ID No: 218 and/or a heavy chain variable region comprising SEQ ID No: 219.

### Embodiment 22

Antibody or binding fragment thereof according to embodiments 1 to 17 comprising a light chain variable region and/or a heavy chain variable region, wherein the light chain variable region comprises at least a CDR1 set forth in SEQ ID No: 226 or sequences at least 80% identical thereto, a CDR2 set forth in SEQ ID No: 227 or sequences at least 80% identical thereto, and/or a CDR3 set forth in SEQ ID No: 228 or sequences at least 80% identical thereto; and/or wherein the heavy chain variable region comprises at least a CDR1 set forth in SEQ ID No: 229 or sequences at least 80% identical thereto, a CDR2 set forth in SEQ ID No: 230 or sequences at least 80% identical thereto, and/or a CDR3 set forth in SEQ ID No: 231 or sequences at least 80% identical thereto.

### Embodiment 23

Antibody or binding fragment thereof according to embodiment 22 comprising a light chain variable region comprising SEQ ID No.: 232 or sequences at least 80% identical thereto and/or a heavy chain variable region comprising SEQ ID No: 233 or sequences at least 80% identical thereto.

### Embodiment 24

Antibody or binding fragment thereof according to embodiment 22 or 23 wherein the antibody comprises a light chain variable region and/or a heavy chain variable region, wherein the light chain variable region comprises at least a CDR1 set forth in SEQ ID No: 226, a CDR2 set forth in SEQ ID No.: 227, and/or a CDR3 set forth in SEQ ID No: 228; and/or wherein the heavy chain variable region comprises at least a CDR1 set forth in SEQ ID No: 229, a CDR2 set forth in SEQ ID No: 230, and/or a CDR3 set forth in SEQ ID No: 231.

### Embodiment 25

Antibody or binding fragment thereof according to embodiment 24 wherein the antibody or binding fragment thereof comprises a light chain variable region comprising SEQ ID No: 232 and/or a heavy chain variable region comprising SEQ ID No: 233.

### Embodiment 26

Antibody or binding fragment thereof according to any of the preceding embodiments wherein the antibody or binding fragment thereof is a monoclonal antibody or binding fragment thereof.

### Embodiment 27

Antibody or binding fragment thereof according to any of the preceding embodiments wherein the monoclonal antibody or binding fragments thereof is chimeric, humanized or human antibody or binding fragment thereof.

### Embodiment 28

Use of an antibody or binding fragment thereof according to any one of the preceding embodiments for depleting a subpopulation of T cells expressing a fraction of TCR Vα chains comprising at least two different TCR Vα chains or for depleting a subpopulation of T cells expressing a fraction of TCR Vβ chains comprising at least two different TCR Vβ chains.

### Embodiment 29

Antibody or binding fragment thereof according to any one of the preceding embodiments for use as a medicament.

### Embodiment 30

Antibody or binding fragment thereof according to any one of the preceding embodiments for use in the treatment of T cell leukemia.

1. An antibody or binding fragment thereof that binds to a fraction of T cell receptor variable alpha (TCR Vα) chains comprising at least two different TCR Vα chains but less than all TCR Vα chains or that binds to a fraction of T cell receptor variable beta (TCR Vβ) chains comprising at least two different TCR Vβ chains but less than all TCR Vβ chains.

2. The antibody or binding fragment thereof according to claim 1, wherein the fraction of TCR Vα chains comprises at least two different TCR Vα chains that belong to at least two different TCR Vα chain subfamilies, or wherein the fraction of TCR Vβ chains comprises at least two different TCR Vβ chains that belong to at least two different TCR Vβ chain subfamilies.

3. The antibody or binding fragment thereof according to claim 1, wherein the antibody or binding fragment thereof binds to a fraction of TCR Vβ chains.

4. The antibody or binding fragment thereof according to claim 3, wherein the fraction comprises at least two different TCR Vβ chains that belong to at least two different subfamilies selected from the group consisting of BV7, BV12, BV 25 and BV 28.

5. The antibody or binding fragment thereof according to claim 4, wherein the fraction comprises at least 2 different TCR Vβ chains selected from the group consisting of TRBV7-3, TRBV12-3, TRBV12-4, TRBV25-1, and TRBV28.

6. The antibody or binding fragment thereof according to claim 4, wherein the fraction comprises at least 2 different TCR Vβ chains selected from the group consisting of TRBV7-2, TRBV7-3, TRBV12-3, TRBV 12-4, TRBV25-1, and TRBV28.

7. The antibody or binding fragment thereof according to claim 3, wherein the fraction comprises at least 2 different TCR Vβ chains selected from the group consisting of TRBV2, TRBV5-6, TRBV6-5, TRBV7-3, TRBV7-9, TRBV10-3, TRBV12-3, TRBV 12-4, TRBC 12-5, TRBV21-1, TRBV6-7, TRBV24-1, TRBV 25-1, and TRBV28.

8. The antibody or binding fragment thereof according to claim 1, wherein the antibody or binding fragment thereof is capable of depleting a subpopulation of T cells expressing a fraction of TCR Vα chains comprising at least two different TCR Vα chains or is capable of depleting a subpopulation of T cells expressing a fraction of TCR Vβ chains comprising at least two different TCR Vβ chains.

9. The antibody or binding fragment thereof according to claim 1 comprising a light chain variable region and/or a heavy chain variable region, wherein the light chain variable region comprises a CDR1 set forth in SEQ ID No: 212 or a sequence at least 80% identical thereto, a CDR2 set forth in SEQ ID No: 213 or a sequence at least 80% identical thereto, or a CDR3 set forth in SEQ ID No: 214 or a sequence at least 80% identical thereto, and combinations thereof; and/or wherein the heavy chain variable region comprises a CDR1 set forth in SEQ ID No: 215 or a sequence at least 80% identical thereto, a CDR2 set forth in SEQ ID No: 216 or a sequence at least 80% identical thereto, or a CDR3 set forth in SEQ ID No: 217 or a sequence at least 80% identical thereto, and combinations thereof.

10. The antibody or binding fragment thereof according to claim 9 comprising a light chain variable region and a heavy chain variable region, wherein the light chain variable region comprises a CDR1 set forth in SEQ ID No: 212, a CDR2 set forth in SEQ ID No: 213, or a CDR3 set forth in SEQ ID No: 214, and combinations thereof; and wherein the heavy chain variable region comprises a CDR1 set forth in SEQ ID No: 215, a CDR2 set forth in SEQ ID No: 216, or a CDR3 set forth in SEQ ID No: 217, and combinations thereof.

11. The antibody or binding fragment thereof according to claim 9 comprising a light chain variable region comprising SEQ ID No: 218 or a sequence at least 80% identical thereto and/or a heavy chain variable region comprising SEQ ID No: 219 or a sequence at least 80% identical thereto.

12. The antibody or binding fragment thereof according to claim 11 wherein the antibody or binding fragment thereof comprises a light chain variable region comprising SEQ ID No: 218 and/or a heavy chain variable region comprising SEQ ID No: 219.

13. The antibody or binding fragment thereof according to claim 1 comprising a light chain variable region and/or a heavy chain variable region, wherein the light chain variable region comprises a CDR1 set forth in SEQ ID No: 226 or a sequence at least 80% identical thereto, a CDR2 set forth in SEQ ID No: 227 or a sequence at least 80% identical thereto, or a CDR3 set forth in SEQ ID No: 228 or a sequence at least 80% identical thereto, and combinations thereof; and/or wherein the heavy chain variable region comprises a CDR1 set forth in SEQ ID No: 229 or a sequence at least 80% identical thereto, a CDR2 set forth in SEQ ID No: 230 or a sequence at least 80% identical thereto, or a CDR3 set forth in SEQ ID No: 231 or a sequence at least 80% identical thereto, and combinations thereof.

14. The antibody or binding fragment thereof according to claim 13 comprising a light chain variable region and a heavy chain variable region, wherein the light chain variable region comprises a CDR1 set forth in SEQ ID No: 226, a CDR2 set forth in SEQ ID No: 227, and a CDR3 set forth in SEQ ID No: 228; and wherein the heavy chain variable region comprises a CDR1 set forth in SEQ ID No: 229, a CDR2 set forth in SEQ ID No.: 230, and a CDR3 set forth in SEQ ID No: 231.

15. The antibody or binding fragment thereof according to claim 13 comprising a light chain variable region set forth in SEQ ID No.: 232 or a sequence at least 80% identical thereto and/or a heavy chain variable region set forth in SEQ ID No: 233 or a sequence at least 80% identical thereto.

16.-17. (canceled)

18. A method of treating T cell leukemia in a human or non-human animal in need thereof, comprising administering an antibody or binding fragment thereof to said human or non-human animal, wherein said antibody or binding fragment thereof binds to a fraction of T cell receptor variable alpha (TCR Vα) chains comprising at least two different TCR Vα chains but less than all TCR Vα chains, or binds to a fraction of T cell receptor variable beta (TCR Vβ) chains comprising at least two different TCR Vβ chains but less than all TCR Vβ chains.

19. The method of claim 18, wherein the fraction of TCR Vα chains comprises at least two different TCR Vα chains that belong to at least two different TCR Vα chain subfamilies, or wherein the fraction of TCR Vβ chains comprises at least two different TCR Vβ chains that belong to at least two different TCR Vβ chain subfamilies.

20. The method of claim 19, wherein said antibody or binding fragment comprises a light chain variable region and a heavy chain variable region, wherein the light chain variable region comprises a CDR1 set forth in SEQ ID No: 212 or a sequence at least 80% identical thereto, a CDR2 set forth in SEQ ID No: 213 or a sequence at least 80% identical thereto, and a CDR3 set forth in SEQ ID No: 214 or a sequence at least 80% identical thereto; and wherein the heavy chain variable region comprises a CDR1 set forth in SEQ ID No: 215 or a sequence at least 80% identical thereto, a CDR2 set forth in SEQ ID No: 216 or a sequence at least 80% identical thereto, and a CDR3 set forth in SEQ ID No: 217 or a sequence at least 80% identical thereto.

21. The method of claim 20, wherein the light chain variable region of the antibody or binding fragment thereof comprises a CDR1 set forth in SEQ ID No: 212, a CDR2 set forth in SEQ ID No: 213, and a CDR3 set forth in SEQ ID No: 214; and wherein the heavy chain variable region of the antibody or binding fragment thereof comprises a CDR1I set forth in SEQ ID No: 215, a CDR2 set forth in SEQ ID No: 216, and a CDR3 set forth in SEQ ID No: 217.
